(19) United States
(12) Patent Application Publication
(10) Pub. No.: US 2024/0400715 A1
(43) Pub. Date: Dec. 5, 2024

(54) MULTISPECIFIC ANTIBODIES AND USES THEREOF

(71) Applicant: Vibrant Pharma Limited, Guangzhou (CN)

(72) Inventors: Changhua Zhou, Guangzhou (CN); Luquan Wang, Guangzhou (CN); Jianbing Zhang, Guangzhou (CN)

(21) Appl. No.: 18/698,853

(22) PCT Filed: Nov. 7, 2022
(86) PCT No.: PCT/CN2022/130399

§ 371 Date: Apr. 5, 2024

(30) Foreign Application Priority Data
Nov. 5, 2021 (WO) PCT/CN2021/129116

(51) Int. Cl. C07K 16/30; A61K 39/00; A61K 47/68; A61P 35/00; C07K 16/28; C07K 16/32

(52) U.S. Cl. CPC C07K 16/3007; A61K 47/68; A61P 35/00; C07K 16/2809; C07K 16/2863; C07K 16/32; A61K 2039/505; C07K 2317/24; C07K 2317/31; C07K 2317/522; C07K 2317/524; C07K 2317/526; C07K 2317/53; C07K 2317/55; C07K 2317/569; C07K 2317/64; C07K 2317/73; C07K 2317/92; C07K 2317/94

(57) ABSTRACT

Provided are multispecific antibodies (e.g., bispecific antibodies) or antigen-binding fragments thereof. In one aspect, the multispecific antibodies or antigen-binding fragments thereof can bind to a T cell antigen (e.g., CD3) and/or a tumor-associated antigen (e.g., CEACAM5), or a combination thereof.

### TECHNICAL FIELD

This disclosure relates to multispecific antibodies or antigen-binding fragments thereof.

### BACKGROUND

Naturally occurring antibodies typically only target one antigen. A multispecific antibody can be manufactured in different structural formats, so that they can simultaneously bind to two or more different epitopes. These epitopes can be in the same antigen or in different antigen. This opens up a wide range of applications, including redirecting T cells to tumor cells, blocking two different signaling pathways simultaneously, dual targeting of different disease mediators, and delivering payloads to targeted sites.

Multispecific antibodies have various applications. However, in some cases, a multispecific antibody may not have the desired efficacy and it can be difficult to express and purify. There is a need to continue to develop various therapeutics based on multispecific antibodies.

### SUMMARY

This disclosure relates to multispecific antibodies or antigen-binding fragments thereof, wherein the multispecific antibodies or antigen-binding fragments thereof specifically bind to a T cell antigen (e.g., CD3) and/or a tumor-associated antigen (e.g., CEACAM5), or a combination thereof.

In one aspect, the disclosure is related to an antigen-binding protein, comprising (a) a Fc; (b) a Fab fragment (Fab) that specifically binds to a T cell antigen; and (c) a single-domain antibody variable domain (VHH) that specifically binds to a tumor-associated antigen. In some embodiments, the Fab and the VHH are linked to the Fc.

In some embodiments, the Fab comprises or consists of a light chain variable domain (VL), a light chain constant domain (CL), a heavy chain variable domain (VH), and a heavy chain first constant domain (CH1).

In some embodiments, the Fab can activate T cells upon binding to the T cell antigen.

In some embodiments, the T cell antigen is cluster of differentiation 3 (CD3).

In some embodiments, the tumor-associated antigen is cluster of differentiate 20 (CD20), prostate-specific antigen (PSA), prostate stem cell antigen (PSCA), programmed death-ligand 1 (PD-L1), human epidermal growth factor receptor 2 (Her2), human epidermal growth factor receptor 3 (Her3), human epidermal growth factor receptor (Her1), β-Catenin, cluster of differentiate 19 (CD19), epidermal growth factor receptor (EGFR), tyrosine-protein kinase Met (c-Met), epithelial cell adhesion molecule (EPCAM), prostate-specific membrane antigen (PSMA), cluster of differentiate 40 (CD40), Mucin 1, Cell Surface Associated (MUC1), insulin-like growth factor 1 receptor (IGF1R), or carcinoembryonic antigen cell adhesion molecule 5 (CEACAM5).

In some embodiments, the Fc is human IgG4 Fc.

In some embodiments, the CH1 domain of the Fab is linked to a CH2 domain in the Fc, optionally via a hinge region.

In some embodiments, the hinge region is a human IgG4 hinge region optionally with S228P mutation according to EU numbering.

In some embodiments, the Fab is linked to the C-terminus of a CH3 domain in the Fc.

In some embodiments, the Fab is linked to the CH3 domain via a linker peptide.

In some embodiments, the VHH is linked to a CH2 domain in the Fc, optionally via a hinge region. In some embodiments, the hinge region is a human IgG4 hinge region optionally with S228P mutation according to EU numbering.

In some embodiments, the VHH is linked to the C-terminus of a CH3 domain in the Fc.

In some embodiments, the VHH is linked to the CH3 domain via a linker peptide.

In some embodiments, the Fc comprises a first polypeptide and a second polypeptide.

In some embodiments, each polypeptide comprises one or more knobs-into-holes mutations.

In one aspect, the disclosure is related to a protein complex, comprising: (a) a first polypeptide comprising from N-terminus to C-terminus: a VH, a CH1 domain, optionally a first hinge region, a first CH2 domain, and a first CH3 domain; (b) a second polypeptide comprising from N-terminus to C-terminus: a VHH, optionally a second hinge region, a second CH2 domain, and a second CH3 domain; (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 11; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: a VH, a CH1 domain, optionally a first hinge region, a first CH2 domain, a first CH3 domain, optionally a linker peptide, and a single-domain antibody variable domain (VHH); (b) a second polypeptide comprising from N-terminus to C-terminus: optionally a second hinge region, a second CH2 domain, and a second CH3 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 6; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: a VH, a CH1 domain, optionally a first hinge region, a first CH2 domain, and a first CH3 domain; (b) a second polypeptide comprising from N-terminus to C-terminus: optionally a second hinge region, a second CH2 domain, a second CH3 domain, optionally a linker peptide, and a single-domain antibody variable domain (VHH); and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 12; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: a single-domain antibody variable domain (VHH), optionally a first hinge region, a first CH2 domain, and a first CH3 domain; (b) a second polypeptide comprising from N-terminus to C-terminus: a VH, a CH1 domain, optionally a second hinge region, a second CH2 domain, and a second CH3 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 4; the second polypeptide comprises a sequence that is at least 80%, 90%, 95%, or 100% identical to SEQ ID NO: 9; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: optionally a first hinge region, a first CH2 domain, a first CH3 domain, optionally a linker peptide, and a single-domain antibody variable domain (VHH); (b) a second polypeptide comprising from N-terminus to C-terminus: a VH, a CH1 domain, optionally a second hinge region, a second CH2 domain, and a second CH3 domain; (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 5; the second polypeptide comprises a sequence that is at least 80%, 90%, 95%, or 100% identical to SEQ ID NO: 9; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: optionally a first hinge region, a first CH2 domain, and a first CH3 domain; (b) a second polypeptide comprising from N-terminus to C-terminus: a VH, a CH1 domain, optionally a second hinge region, a second CH2 domain, a second CH3 domain, optionally a linker peptide, and a single-domain antibody variable domain (VHH); and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 13; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: a single-domain antibody variable domain (VHH), optionally a first hinge region, a first CH2 domain, a first CH3 domain, optionally a linker peptide, a VH, and a CH1 domain; (b) a second polypeptide comprising from N-terminus to C-terminus: optionally a second hinge region, a second CH2 domain, and a second CH3 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 7; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: optionally a first hinge region, a first CH2 domain, a first CH3 domain, optionally a linker peptide, a VH, and a CH1 domain; (b) a second polypeptide comprising from N-terminus to C-terminus: a single-domain antibody variable domain (VHH), optionally a second hinge region, a second CH2 domain, and a second CH3 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 3; the second polypeptide comprises a sequence that is at least 80%, 90%, 95%, or 100% identical to SEQ ID NO: 11; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: optionally a first hinge region, a first CH2 domain, a first CH3 domain, optionally a first linker peptide, a VH, and a CH1 domain; (b) a second polypeptide comprising from N-terminus to C-terminus: optionally a second hinge region, a second CH2 domain, a second CH3 domain, optionally a second linker peptide, and a single-domain antibody variable domain (VHH); and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 3; the second polypeptide comprises a sequence that is at least 80%, 90%, 95%, or 100% identical to SEQ ID NO: 12; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: a single-domain antibody variable domain (VHH), optionally a first hinge region, a first CH2 domain, and a first CH3 domain; (b) a second polypeptide comprising from N-terminus to C-terminus: optionally a second hinge region, a second CH2 domain, a second CH3 domain, optionally a linker peptide, a VH, and a CH1 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 4; the second polypeptide comprises a sequence that is at least 80%, 90%, 95%, or 100% identical to SEQ ID NO: 10; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: optionally a first hinge region, a first CH2 domain, and a first CH3 domain; (b) a second polypeptide comprising from N-terminus to C-terminus: a single-domain antibody variable domain (VHH), optionally a second hinge region, a second CH2 domain, a second CH3 domain, optionally a linker peptide, a VH, and a CH1 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 14; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: optionally a first hinge region, a first CH2 domain, a first CH3 domain, optionally a first linker peptide, and a single-domain antibody variable domain (VHH); (b) a second polypeptide comprising from N-terminus to C-terminus: optionally a second hinge region, a second CH2 domain, a second CH3 domain, optionally a second linker peptide, a VH, and a CH1 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 5; the second polypeptide comprises a sequence that is at least 80%, 90%, 95%, or 100% identical to SEQ ID NO: 10; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: a VH, a CH1 domain, optionally a first hinge region, a first CH2 domain, and a first CH3 domain; (b) a second polypeptide comprising from N-terminus to C-terminus: optionally a second hinge region, a second CH2 domain, and a second CH3 domain; and (c) a third polypeptide comprising from N-terminus to C-terminus: a VL, a CL domain, optionally a third linker peptide, a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 39.

In one aspect, the disclosure is related to a protein complex, comprising: (a) a first polypeptide comprising from N-terminus to C-terminus: optionally a first hinge region, a first CH2 domain, and a first CH3 domain; (b) a second polypeptide comprising from N-terminus to C-terminus: a VH, a CH1 domain, optionally a second hinge region, a second CH2 domain, and a second CH3 domain; and (c) a third polypeptide comprising from N-terminus to C-terminus: a VL, a CL domain, optionally a third linker peptide, and a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 9; and the third polypeptide comprises a sequence that is at least 80%, 90%, 95%, or 100% identical to SEQ ID NO: 39.

In one aspect, the disclosure is related to a protein complex, comprising: (a) a first polypeptide comprising from N-terminus to C-terminus: a VH, a CH1 domain, optionally a first hinge region, a first CH2 domain, and a first CH3 domain; (b) a second polypeptide comprising from N-terminus to C-terminus: optionally a second hinge region, a second CH2 domain, and a second CH3 domain; and (c) a third polypeptide comprising from N-terminus to C-terminus: a single-domain antibody variable domain (VHH), optionally a third linker peptide, a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 40.

In one aspect, the disclosure is related to a protein complex, comprising: (a) a first polypeptide comprising from N-terminus to C-terminus: optionally a first hinge region, a first CH2 domain, and a first CH3 domain; (b) a second polypeptide comprising from N-terminus to C-terminus: a VH, a CH1 domain, optionally a second hinge region, a second CH2 domain, and a second CH3 domain; and (c) a third polypeptide comprising from N-terminus to C-terminus: a single-domain antibody variable domain (VHH), optionally a linker peptide, a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 9; and the third polypeptide comprises a sequence that is at least 80%, 90%, 95%, or 100% identical to SEQ ID NO: 40.

In one aspect, the disclosure is related to a protein complex, comprising: (a) a first polypeptide comprising from N-terminus to C-terminus: a single-domain antibody variable domain (VHH), optionally a first linker peptide, a VH, a CH1 domain, optionally a first hinge region, a first CH2 domain, and a first CH3 domain; (b) a second polypeptide comprising from N-terminus to C-terminus: optionally a second hinge region, a second CH2 domain, and a second CH3 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 41; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: optionally a first hinge region, a first CH2 domain, and a first CH3 domain; (b) a second polypeptide comprising from N-terminus to C-terminus: a single-domain antibody variable domain (VHH), optionally a second linker peptide, a VH, a CH1 domain, optionally a second hinge region, a second CH2 domain, and a second CH3 domain; and (c) a third polypeptide comprising from N-terminus to C-terminus: a VL, and a CL domain. In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 42; and the third polypeptide comprises a sequence that is at least 80%, 90%, 95%, or 100% identical to SEQ ID NO: 23.

In one aspect, the disclosure is related to a protein complex, comprising: (a) a first polypeptide comprising from N-terminus to C-terminus: a VH, a CH1 domain, optionally a first hinge region, a first CH2 domain, a first CH3 domain, optionally a first linker peptide, and a single-domain antibody variable domain (VHH); and (b) a second polypeptide comprising from N-terminus to C-terminus: a VL, a CL, optionally a second hinge region, a second CH2 domain, and a second CH3 domain. In some embodiments, the VH and the VL associate with each other, forming an antigen binding site of a Fab that specifically binds to a T cell antigen, In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 6; and the second polypeptide comprises a sequence that is at least 80%, 90%, 95%, or 100% identical to SEQ ID NO: 44.

In one aspect, the disclosure is related to a protein complex, comprising: (a) a first polypeptide comprising from N-terminus to C-terminus: a VH, a CH1 domain, optionally a first hinge region, a first CH2 domain, and a first CH3 domain; and (b) a second polypeptide comprising from N-terminus to C-terminus: a VL, a CL, optionally a second hinge region, a second CH2 domain, a second CH3 domain, optionally a second linker peptide, and a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 2; and the second polypeptide comprises a sequence that is at least 80%, 90%, 95%, or 100% identical to SEQ ID NO: 46.

In one aspect, the disclosure is related to a protein complex, comprising: (a) a first polypeptide comprising from N-terminus to C-terminus: a VL, a CL, optionally a first hinge region, a first CH2 domain, a first CH3 domain, optionally a first linker peptide, and a single-domain antibody variable domain (VHH); and (b) a second polypeptide comprising from N-terminus to C-terminus: a VH, a CH1 domain, optionally a second hinge region, a second CH2 domain, and a second CH3 domain. In some embodiments, the VH and the VL associate with each other, forming an antigen binding site of a Fab that specifically binds to a T cell antigen.

In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 45; and the second polypeptide comprises a sequence that is at least 80%, 90%, 95%, or 100% identical to SEQ ID NO: 9.

In one aspect, the disclosure is related to a protein complex, comprising: (a) a first polypeptide comprising from N-terminus to C-terminus: a VL, a CL, optionally a first hinge region, a first CH2 domain, and a first CH3 domain; and (b) a second polypeptide comprising from N-terminus to C-terminus: a VH, a CH1 domain, optionally a second hinge region, a second CH2 domain, a second CH3 domain, optionally a second linker peptide, and a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen. In some embodiments, the T cell antigen is CD3, and the tumor-associated antigen is CEACAM5.

In some embodiments, the first polypeptide comprises a sequence that is at least 80%, 90%, 95%, or 100% identical to SEQ ID NO: 43; the second polypeptide comprises a sequence that is at least 80%, 90%, 95%, or 100% identical to SEQ ID NO: 13.

In some embodiments, the first CH3 domain comprises one or more knob mutations, and the second CH3 domain comprises one or more hole mutations.

In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38.

In some embodiments, the VHH comprises a sequence that is at least 80%, 90%, 95%, or 100% identical to SEQ ID NO: 18.

In some embodiments, the first hinge region and/or the second hinge region comprise a sequence that is at least 80%, 90%, 95%, or 100% identical to SEQ ID NO: 19.

In some embodiments, the first Fc region and/or the second Fc region comprise a sequence that is at least 80%, 90%, 95%, or 100% identical to SEQ ID NO: 20 or 21.

In some embodiments, the linker peptide, the first linker peptide, the second linker peptide and/or the third linker peptide comprises a sequence that is at least 80%, 90%, 95%, or 100% identical to SEQ ID NO: 15 or one or more repeats (e.g., 1, 2, 3, 4, 5, 6, 7, or 8) of SEQ ID NO: 16.

In one aspect, the disclosure is related to a nucleic acid comprising a polynucleotide encoding the antigen-binding protein or the protein complex described herein. In some embodiments, the nucleic acid is a DNA (e.g., cDNA) or RNA (e.g., mRNA).

In one aspect, the disclosure is related to a vector comprising one or more of the nucleic acids described herein.

In one aspect, the disclosure is related to a cell comprising the vector described herein.

In some embodiments, the cell is a HEK293F cell or CHO cell.

In one aspect, the disclosure is related to a cell comprising one or more of the nucleic acids described herein.

In one aspect, the disclosure is related to a method of producing an antigen-binding protein or protein complex, the method comprising: (a) culturing the cell described herein under conditions sufficient for the cell to produce the antigen-binding protein or protein complex; and (b) collecting the antigen-binding protein or protein complex produced by the cell.

In one aspect, the disclosure is related to an antibody-drug conjugate comprising the antigen-binding protein or the protein complex described herein, covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antigen-binding protein, the protein complex, or the antibody-drug conjugate described herein, to the subject. In some embodiments, the subject has a cancer expressing CEACAM5. In some embodiments, the cancer is lung cancer, colorectal cancer, head and neck cancer, stomach cancer, pancreatic cancer, urothelial cancer, breast cancer, cervical cancer, or endometrial cancer.

In one aspect, the disclosure is related to a method of decreasing the rate of tumor growth, the method comprising: contacting a tumor cell with an effective amount of a composition comprising the antigen-binding protein, the protein complex, or the antibody-drug conjugate described herein.

In one aspect, the disclosure is related to a method of killing a tumor cell, the method comprising: contacting a tumor cell with an effective amount of a composition comprising the antigen-binding protein, the protein complex, or the antibody-drug conjugate described herein.

In one aspect, the disclosure is related to a pharmaceutical composition comprising the antigen-binding protein or the protein complex described herein, and a pharmaceutically acceptable carrier.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multispecific antibodies, bispecific antibodies, single-chain antibodies, diabodies, linear antibodies formed from these antibodies or antibody fragments, and antigen binding protein constructs.

As used herein, the term “antigen-binding fragment” refers to a portion of a full-length antibody, wherein the portion of the antibody is capable of specifically binding to an antigen.

In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)₂, and Fv fragments, scFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general.

An antibody that specifically binds to the target molecule may be referred to as a target-specific antibody. For example, an antibody that specifically binds to CEACAM5 may be referred to as CEACAM5-specific antibody or an anti-CEACAM5 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens.

A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain antibody. In some embodiments, the VHH is a humanized VHH. In some embodiments, the VHH is a single-domain antibody (sdAb).

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1X show schematic structures of several bispecific antibody constructs including a Fab (T cell activator) and a tumor-associated antigen-targeting moiety in the form of sdAb.

FIGS. 2A-2F show images of SDS-PAGE (sodium dodecyl sulphate-polyacrylamide gel electrophoresis) results of the bispecific antibodies. Each bispecific antibody sample was analyzed under either a non-reducing or reducing condition. M is the molecular weight (MW) marker. Names of the bispecific antibodies are labeled over each gel image. Band sizes are also labeled on the left side of each gel image.

FIGS. 3A-3F show the binding of the bispecific antibodies to CEA expressing cells (H1573).

FIGS. 3G-3L show the binding of the bispecific antibodies to CD3 expressing cells (Jurkat).

FIGS. 4A-4Z show T cell-mediated killing of CEA-expressing tumor target cells induced by the bispecific antibodies. The following tumor target cells were tested: H157 cells (FIGS. 4A-4B), H1650 cells (FIGS. 4C-4F), HT-29 cells (FIGS. 4G-4L), H1395 cells (FIGS. 4M-4R), H1573 cells (FIGS. 4S-4V), and BxPC-3 cells (FIGS. 4W-4Z).

FIGS. 5A-5L show cytokine secretion after T cell-mediated killing of CEA-expressing tumor cells induced by 12 bispecific antibodies (101-112).

FIGS. 6A-6T show cytokine secretion after T cell-mediated killing of CEA-expressing tumor cells induced by 10 bispecific antibodies (201-206, 208-210 and 104).

FIGS. 7A-7F show the serum stability of the bispecific antibodies as measured by flow cytometry (FACS).

FIG. 8 shows the SDS-PAGE results of two EGFR-CD3 bispecific antibodies (401 and 404) and two HER2-CD3 bispecific antibodies (501 and 504).

FIGS. 9A-9Z and 10A-10F show T cell-mediated killing of EGFR-expressing or HER2-expressing tumor target cells induced by the EGFR-CD3 bispecific antibodies (401 and 404) and HER2-CD3 bispecific antibodies (501 and 504).

FIG. 11 shows the salt-gradient affinity capture self-interaction nanoparticle spectroscopy (SGAC-SINS) data of the EGFR-CD3 bispecific antibodies (401 and 404) and HER2-CD3 bispecific antibodies (501 and 504).

FIG. 12 shows the in vivo anti-tumor activity of the CEA-CD3 bispecific antibodies (101 and 104) and the HER2-CD3 bispecific antibodies (501 and 504) in PBMC/LS174T inoculated NCG mice.

FIG. 13 shows the body weight of PBMC/LS174T inoculated NCG mice treated with the CEA-CD3 bispecific antibodies (101 and 104) and the HER2-CD3 bispecific antibodies (501 and 504).

FIG. 14 shows the in vivo anti-tumor activity of the EGFR-CD3 bispecific antibodies (401 and 404) in PBMC/HT-29 inoculated NCG mice.

FIG. 15 shows the body weight of PBMC/HT-29 inoculated NCG mice treated with the EGFR-CD3 bispecific antibodies (401 and 404).

FIG. 16 lists some of the amino acid sequences discussed in the present disclosure.

### DETAILED DESCRIPTION

This disclosure relates to multispecific antibodies (e.g., bispecific antibodies) or antigen-binding proteins. In one aspect, the multispecific antibodies or antigen-binding proteins can bind to a T cell antigen (e.g., CD3) and/or a tumor-associated antigen (e.g., CEACAM5), or a combination thereof.

In general, bispecific antibodies or multispecific antibodies include two or more antigen-binding sites targeting different antigens or different epitopes of the same antigen. Thus, bispecific antibodies or multispecific antibodies can have more functions than a monospecific antibody. For example, these functions include, but not limited to, stronger binding to an antigen through an avidity effect; co-localization of bound antigens (e.g., Her2 and Her3) on the cell surface and the effect therefrom; increasing the serum half-life of an antibody fragment by linking it to a second antibody fragment that is bound to a protein with a long serum half-life, e.g., albumin or transferrin; and bringing two cells into proximity by binding to an antigen on each of the cells.

Among the purposes of bispecific antibodies or multispecific antibodies, one class of molecules, T cell engagers (TCE), has gained more attention. A TCE is a bispecific antibody or multispecific antibody which binds to an antigen on a T cell and an antigen on another cell simultaneously. CD3 is usually selected as the antigen on the T cell. A cancer or tumor cell is usually selected as the other cell type as discussed above. Through binding to CD3 on T cells and a tumor associated antigen (TAA) on cancer cells, the TCE can induce activation of T cells upon binding to cancer cells and cause the killing of the latter.

Nevertheless, there are some hurdles to overcome in order to generate desirable homogeneous bispecific antibodies or multispecific antibodies. The first hurdle is mismatch of heavy chains that bind to the same target (e.g., antigen or epitope). For example, to generate bispecific antibodies with a desired format, the heavy chains targeting different targets should ideally form a heterodimer. However, the percentage of the desired bispecific or multispecific antibody varies greatly in different constructs. Mutations to induce the formation of knobs-into-holes between two heavy chains can be employed to prevent the formation of homodimers of the heavy chains that bind to the same target. Exemplary amino acid sequences of knob-chain and hole-chain Fc that facilitate heterodimer formation are set forth in SEQ ID NO: 1 and SEQ ID NO: 8, respectively.

The second hurdle to overcome is the mismatch between heavy chain variable regions (VHs) and light chain variable regions (VLs). A monoclonal antibody has two identical Fab fragments, each having a paired VH and VL. By contrast, a bispecific antibody usually has two different heavy chain variable regions and two different light chain variable regions. Therefore, there is a possibility that each VH can bind to the two VLs and each VL can bind to the two VHs. As a result, only half of the formed bispecific antibodies are functional without addressing this mismatch issue.

Several strategies have been designed to disable this mismatch. One solution is to design an antibody with a common light chain. Specifically, a light chain, or more precisely a VL, is selected which can form a dimer with all VHs. This design abrogated the necessity of matching VH and VL with the same target-binding specificity. The shortcoming of this strategy is that the contribution of VL in target binding is greatly reduced, leading to difficulty finding an optimal VH as the VH will be greatly if not entirely responsible for target binding.

Another solution is the use of CrossMAb technology, in which a VL is fused to heavy chain constant domain 1 (CH1) and becomes part of this heavy chain. Meanwhile, the corresponding VH is fused to the light chain constant region (CL) and becomes a part of this light chain.

The present disclosure provides a different strategy. The reason of employing technologies, e.g., CrossMAb or common light chain, is because conventional antibodies have and need both heavy and light chains to function and/or maintain stability. However, target binding does not necessarily require both heavy and light chains. For example, a Fab fragment (Fab) contains a variable and constant domain of the light chain and a variable domain and the first constant domain (CH1) of the heavy chain, is fully functional for antigen binding. The variable domain of heavy chain antibodies (VHH), e.g., derived from camelids such as llama, camel, or alpaca, is fully functional for antigen binding. By fusing antibody fragments (e.g., Fab or VHH) to human Fc with heterodimer preference, bispecific antibodies can be generated without considering the mismatch between heavy and light chains.

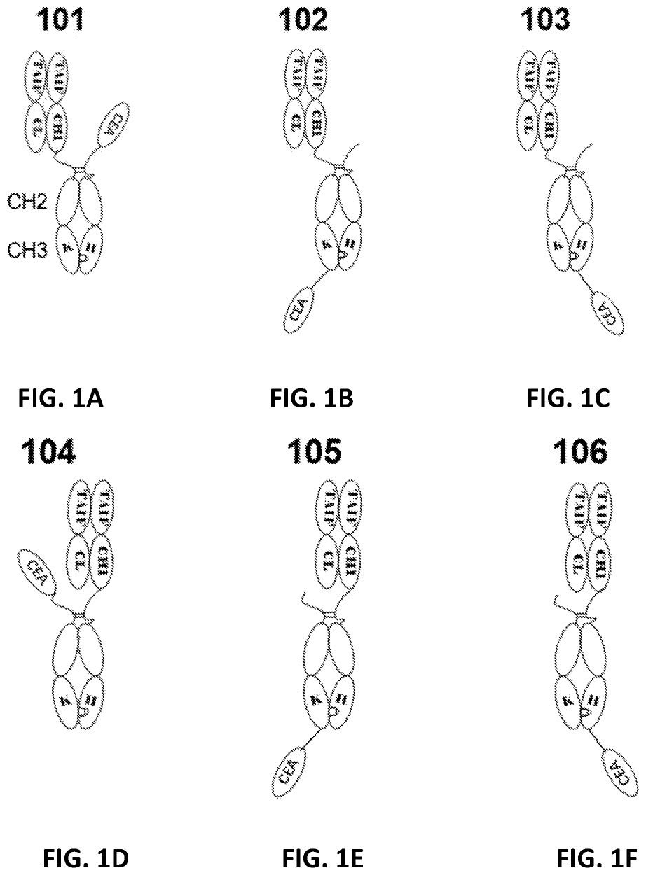

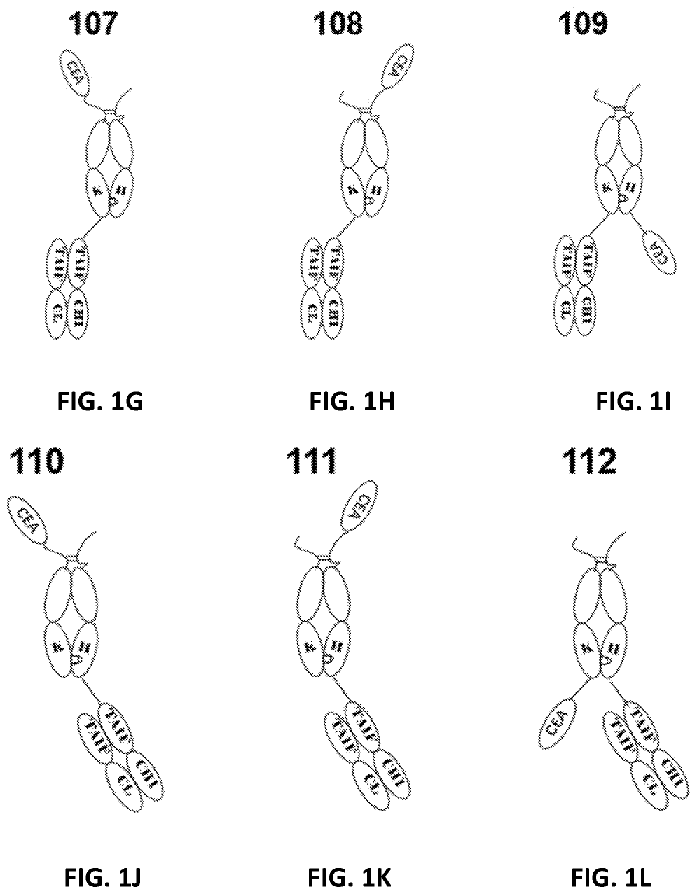

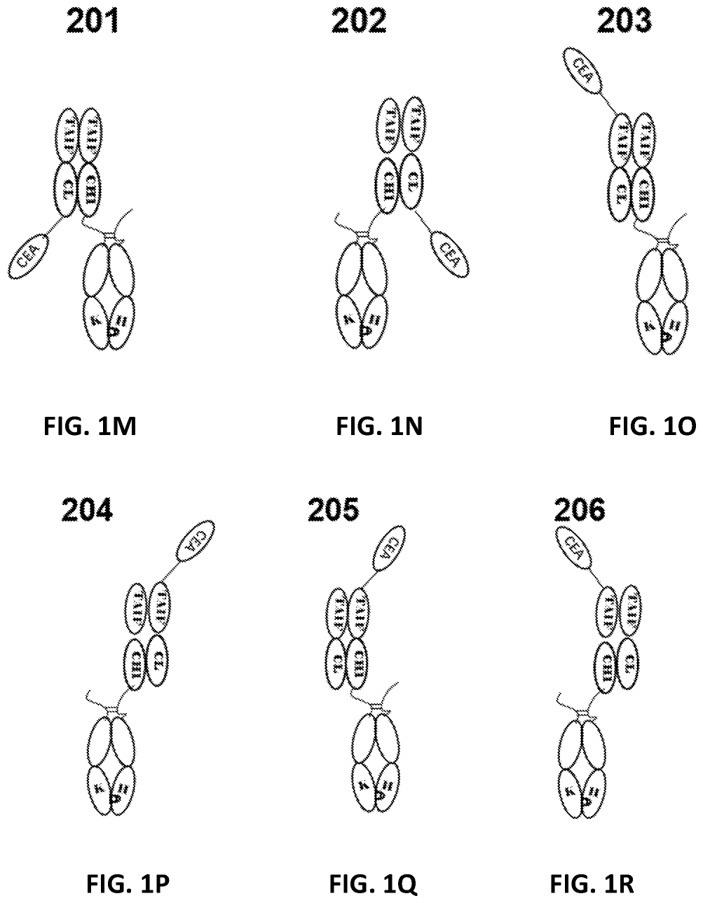

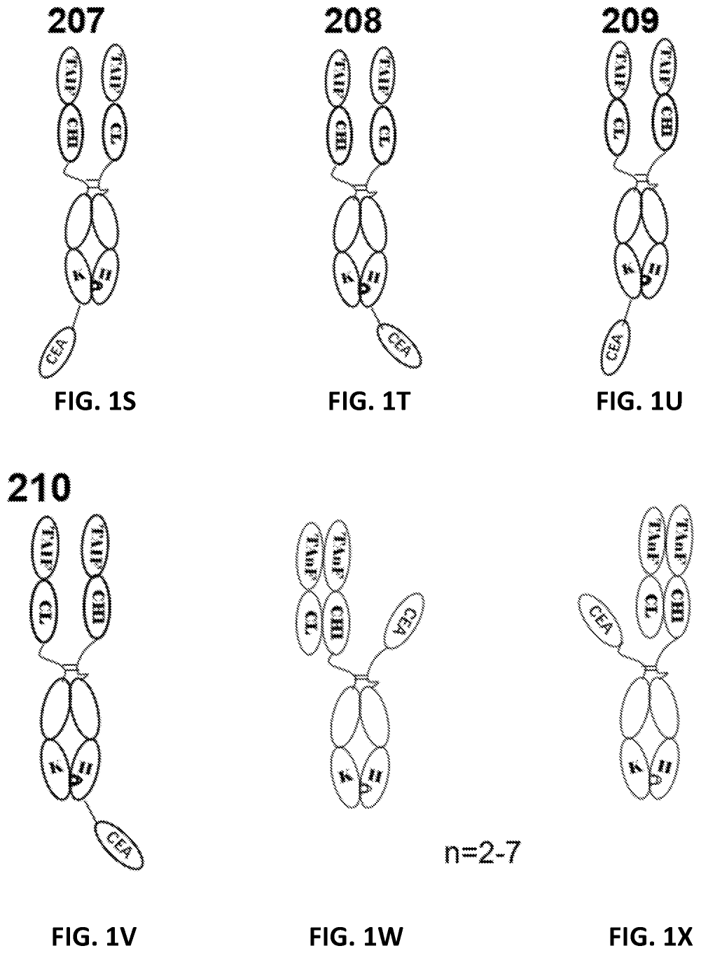

In this disclosure, a Fab that can bind to human CD3 and a VHH that can bind to human carcinoembryonic antigen-related cell adhesion molecule 5 (CEACAM5) is provided. Either antibody fragment is positioned on each of the four ends of Fc (N- or C-end of knob or hole chain), and the other antibody fragment on the other three available ends of the Fc. Such a design gives rise to 12 different molecules (See FIGS. 1A-1L). 10 additional structures are provided in FIGS. 1M-1V. These molecules are provided, characterized for their ability to form heterodimer, and tested for their capability in inducing tumor cell killing in the presence of human PBMCs.

In some embodiments, the multispecific antibody or antigen-binding protein can include 1, 2, 3, 4 or more than four Fab fragments. In some embodiments, the multispecific antibody or antigen-binding protein can include 1, 2, 3, 4, 5 or more than five VHHs. In some embodiments, the Fab can target CD3 or another tumor associated antigen. In some embodiments, the VHH can target CD3 or another tumor associated antigen. The Fab, the VHH, and the multispecific antibody or the antigen binding proteins with various formats are described in detail below.

### Fab Fragment (Fab)

A Fab fragment (Fab) contains a variable and constant domain of the light chain and a variable domain and the first constant domain (CH1) of the heavy chain.

The disclosure provides e.g., anti-CD3 antibodies, the modified antibodies thereof, the chimeric antibodies thereof, and the humanized antibodies thereof. The disclosure also provides Fab fragments that targets CD3. The Fab can be used in various multispecific antibody constructs as described herein.

In some embodiments, the Fab can have a VH that is at least 80%, 85%, 90%, 91%, 92%, 93%, 94%, 95%, 96%, 97%, 98%, or 99% identical to SEQ ID NOs: 25-31 (TA1-TA7).

In some embodiments, the Fab can have a VL that is at least 80%, 85%, 90%, 91%, 92%, 93%, 94%, 95%, 96%, 97%, 98%, or 99% identical to SEQ ID NOs: 32-38 (TA1-TA7).

The amino acid sequences for various Fab fragments are also provided. In some embodiments, the heavy chain portion of the Fab (VH-CH1) is at least 80%, 85%, 90%, 91%, 92%, 93%, 94%, 95%, 96%, 97%, 98%, or 99% identical to SEQ ID NO: 17 (TA1). In some embodiments, the light chain portion of the Fab (VL-CL) is at least 80%, 85%, 90%, 91%, 92%, 93%, 94%, 95%, 96%, 97%, 98%, or 99% identical to SEQ ID NO: 23 (TA1).

In some embodiments, the Fab, the antibody or an antigen-binding fragment described herein can have a heavy chain variable region (VH) comprising VH CDR1, VH CDR2, and VH CDR3 that are identical to VH CDR1, VH CDR2, and VH CDR3 of any VH as described herein (e.g., SEQ ID NOs: 25-31); and a light chain variable region (VL) comprising VL CDR1, VL CDR2, and VL CDR3 that are identical to VL CDR1, VL CDR2, and VL CDR3 of a VL as described herein (e.g., SEQ ID NOs: 32-38).

In some embodiments, the Fab, the antibody or an antigen-binding fragment described herein can contain a VH containing VH CDR1 with zero, one or two amino acid insertions, deletions, or substitutions; VH CDR2 with zero, one or two amino acid insertions, deletions, or substitutions; VH CDR3 with zero, one or two amino acid insertions, deletions, or substitutions, and a VL containing one, two, or three of VL CDR1 with zero, one or two amino acid insertions, deletions, or substitutions; VL CDR2 with zero, one or two amino acid insertions, deletions, or substitutions; VL CDR3 with zero, one or two amino acid insertions, deletions, or substitutions, wherein the VH CDRs are selected from any VH as described herein, and the VL CDRs are selected from any VL as described herein.

Heavy-chain antibody variable domain (VHH) Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

The disclosure provides e.g., anti-CEACAM5 antibodies, the modified antibodies thereof, the chimeric antibodies thereof, and the humanized antibodies thereof. The disclosure also provides VHH of these antibodies. These VHHs can be used in various multispecific antibody constructs as described herein.

The amino acid sequences for various VHH are also provided. In some embodiments, the VHH domain is at least 80%, 85%, 90%, 91%, 92%, 93%, 94%, 95%, 96%, 97%, 98%, or 99% identical to SEQ ID NO: 18.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs of SEQ ID NO: 18.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to CEACAM5. The antibodies or antigen-binding fragments thereof contain a heavy-chain antibody variable domain (VHH) comprising or consisting of an amino acid sequence that is at least 80%, 85%, 90%, or 95% identical to SEQ ID NO: 18.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished, e.g., using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy-chain antibody variable domain (VHH).

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

### Structures of Multispecific Antibodies

The multispecific antibodies (e.g., bispecific antibodies) can be designed to include one or more antigen-binding sites that target T cell antigens (e.g., CD3, CD4, and CD8), and include one or more antigen-binding sites that target a tumor-associated antigen (e.g., CEACAM5). The antigen-binding site can comprise e.g., a Fab, a scFv, a VHH. In some embodiments, the one or more antigen-binding sites that target a T cell antigen (e.g., CD3) can comprise a Fab. In some embodiments, the one or more antigen-binding sites that target the tumor-associated antigen can comprise a VHH. The tumor-associated antigen refers to an antigen that is specifically expressed on tumor cell surfaces. These antigens can be used to identify tumor cells. Normal cells rarely express these tumor associated antigens. Some exemplary tumor-associated antigens include, e.g., CD20, PSA, PSCA, PD-L1, Her2, Her3, Her1, β-Catenin, CD19, CEACAM5, EGFR, c-Met, EPCAM, PSMA, CD40, MUC1, and IGF1R, etc.

In some embodiments, a multispecific antibody (e.g., a bispecific antibody) or antigen-binding fragment thereof described herein includes a Fab that specifically binds to a T cell antigen. In some embodiments, the T cell antigen is CD3 (e.g., human CD3). In some embodiments, the T cell antigen is CD28. In some embodiments, the T cell antigen is CD27. In some embodiments, the T cell antigen is CD137. In some embodiments, the T cell antigen is OX40. In some embodiments, the T cell antigen is PD1. In some embodiments, the T cell antigen is CTLA-4. In some embodiments, the T cell antigen is Tim3. In some embodiments, the T cell antigen is LAG-3. In some embodiments, the multispecific antibody or antigen-binding fragment thereof can activate T cells upon binding to the T cell antigen.

In some embodiments, a multispecific antibody (e.g., a bispecific antibody) or antigen-binding fragment thereof described herein includes a VHH that specifically binds to a tumor-associated antigen. In some embodiments, the tumor-associated antigen is CEACAM5 (e.g., human CEACAM5). In some embodiments, the tumor-associated antigen is CEACAM6. In some embodiments, the tumor-associated antigen is EGFR or Her2. In some embodiments, the tumor-associated antigen is EGFR or Her2. In some embodiments, the tumor-associated antigen is Claudin18.2. In some embodiments, the tumor-associated antigen is CD166. In some embodiments, the tumor-associated antigen is Glypican-3. These are just examples of tumor-associated antigens. Listing of them does not mean to limit the utility of only these antigens.

The present disclosure provides antigen-binding protein constructs with various formats as described herein. While not intending to be bound by any theory, it is hypothesized that the in the presence of the target cells (e.g., cancer cells) and T cells, the protein constructs can effectively activate T cells.

In some embodiments, the multispecific antibodies (e.g., bispecific antibodies) are designed to include a Fab that targets CD3. In some embodiments, the multispecific antibodies (e.g., bispecific antibodies) are designed to include a VHH that targets CEACAM5. The multispecific antibodies are described below.

CD3 (cluster of differentiation 3) is a protein complex and T cell co-receptor that is involved in activating both the cytotoxic T cell (CD8+ naive T cells) and T helper cells (CD4+ naive T cells). It is composed of four distinct chains. In mammals, the complex contains CD3γ chain, CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex. In some embodiments, the multispecific antibodies target CD3ε.

CEACAM5 (carcinoembryonic antigen-related cell adhesion molecule 5) is a cell surface glycoprotein that represents the founding member of the carcinoembryonic antigen (CEA) family of proteins. It is used as a clinical biomarker for gastrointestinal cancers and may promote tumor development through its role as a cell adhesion molecule. Additionally, CEACAM5 may regulate differentiation, apoptosis, and cell polarity.

The present disclosure provides multispecific antibodies (e.g., bispecific antibodies) that bind to both a T cell antigen (e.g., CD3) and a tumor associated antigen. The multispecific antibodies can be used to treat tumor associated antigen positive cancers in a subject (e.g., a human patient). In some embodiments, the tumor associated antigen positive cancer is CEACAM5-positive (e.g., lung cancer, colorectal cancer, head and neck cancer, stomach cancer, pancreatic cancer, urothelial cancer, breast cancer, cervical cancer, or endometrial cancer).

In general, the multispecific antibody (e.g., bispecific antibody) described herein can be prepared, which includes (a) a first polypeptide including a first Fc region (e.g., CH2 domain and CH3 domain); and (b) a second polypeptide including a second Fc region (e.g., CH2 domain and CH3 domain). In some embodiments, the first Fc region and/or the second Fc region are derived from human IgG4. In some embodiments, the first Fc region includes a sequence that is at least 80%, 85%, 90%, 95%, 96%, 97%, 98%, 99%, or 100% identical to SEQ ID NO: 20. In some embodiments, the second Fc region includes a sequence that is at least 80%, 85%, 90%, 95%, 96%, 97%, 98%, 99%, or 100% identical to SEQ ID NO: 21. In some embodiments, the first Fc region and/or the second Fc region include one or more knobs-into-holes mutations. For example, the first Fc region (e.g., the CH3 domain in the Fc region) can include a tryptophan (Trp) at position 366 according to EU numbering; and the second Fc region (e.g., the CH3 domain in the Fc region) can include one or more of the following a serine (Ser) at position 366, an alanine (Ala) at position 368, and/or a valine (Val) at position 407 according to EU numbering.

In some embodiments, the Fc region is derived from the Fc of any antibody as described herein (e.g., IgG1, IgG2, IgG3, and IgG4). In some embodiments, the Fc region is a human IgG1, IgG2, or IgG4 (e.g., a human IgG4). In some embodiments, the first Fc region and/or the second Fc region include additional mutations relative to the Fc region of a wild-type human IgG (e.g., IgG4). For example, the first Fc region and/or the second Fc region can include a proline (Pro) at position 228 according to EU numbering, to reduce chain exchange of the multispecific antibody. The first Fc region and/or the second Fc region can also include an alanine (Ala) at positions 234 according to EU numbering, to reduce ADCC effect of the multispecific antibody. The first Fc region can include a cysteine (Cys) at position 354 and the second Fc region can further include a cysteine (Cys) at position 349 according to EU numbering, to stabilize the multispecific antibody. The second Fc region can include a lysine (Lys) at position 435 and/or a phenylalanine (Phe) at position 436 according to EU numbering, to reduce binding of the second polypeptide to Protein A. In addition, to improve antibody stability, a glycine (Gly) at position 446 and/or a lysine (Lys) at position 447 of the first Fc region and/or the second Fc region can be deleted. While not intending to be bound by any theory, it is understood by a person skilled in the art that the mutations and deletions described herein can be introduced in either the first Fc region or the second Fc region.

In one aspect, the disclosure is related to an antigen-binding protein, comprising (a) a Fc; (b) a first antigen-binding site comprising a Fab that specifically binds to CD3; and (c) a second antigen-binding site comprising a single-domain antibody variable domain (VHH) that specifically binds to CEACAM5, in some embodiments, the first antigen-binding site and the second antigen-binding site are linked to the Fc.

In some embodiments, the first antigen-binding site comprises a Fab fragment (Fab) that contains a variable and constant domain of the light chain and a variable domain and the first constant domain (CH1) of the heavy chain. In some embodiments, the Fab can activate T cells upon binding to the CD3. In some embodiments, the Fab is human IgG4 Fab.

In some embodiments, the Fab is linked to a CH2 domain in the Fc, optionally via a hinge region. In some embodiments, the hinge region is a human IgG4 hinge region optionally with S228P mutation according to EU numbering.

In some embodiments, the Fab is linked to the C-terminus of a CH3 domain in the Fc. In some embodiments, the Fab is linked to the CH3 domain via a linker peptide.

In some embodiments, the VHH is linked to a CH2 domain in the Fc, optionally via a hinge region. In some embodiments, the hinge region is a human IgG4 hinge region optionally with S228P mutation according to EU numbering.

In some embodiments, the VHH is linked to the C-terminus of a CH3 domain in the Fc. In some embodiments, the VHH is linked to the CH3 domain via a linker peptide.

In some embodiments, the Fc comprises a first polypeptide chain and a second polypeptide chain, in some embodiments, each chain comprises one or more knobs-into-holes mutations.

In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38.

In some embodiments, the hinge region comprise a sequence that is at least 80%, 90%, 95%, or 100% identical to SEQ ID NO: 19.

In some embodiments, the Fc region comprise a sequence that is at least 80%, 90%, 95%, or 100% identical to SEQ ID NO: 1, 8, 20 or 21.

In some embodiments, the CH1 domain comprise a sequence that is at least 80%, 90%, 95%, or 100% identical to SEQ ID NO: 22.

In some embodiments, the linker comprises a sequence that is at least 80%, 90%, 95%, or 100% identical to SEQ ID NO: 15 or one or more repeats (e.g., 1, 2, 3, 4, 5, 6, 7, or 8) of SEQ ID NO: 16.

The multispecific antibodies with various structures are described below.

### 1. Fab-Knob+CEA-Hole (101)

As shown in FIG. 1A (101) and FIG. 1W (301-306), a multispecific antibody (e.g., bispecific antibody) can be prepared, which includes (a) a first polypeptide including, preferably from N-terminus to C-terminus: a VH, a CH1 domain, optionally a first hinge region, and a first Fc region (e.g., CH2 domain and CH3 domain); (b) a second polypeptide including, preferably from N-terminus to C-terminus: a single-domain antibody variable domain (VHH), optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19.

In some embodiments, the first polypeptide comprises a sequence that is at least 80%, 85%, 90%, 95%, 96%, 97%, 98%, 99%, or 100% identical to SEQ ID NO: 2. In some embodiments, the second polypeptide comprises a sequence that is at least 80%, 85%, 90%, 95%, 96%, 97%, 98%, 99%, or 100% identical to SEQ ID NO: 11. In some embodiments, the third polypeptide comprises a sequence that is at least 80%, 85%, 90%, 95%, 96%, 97%, 98%, 99%, or 100% identical to SEQ ID NO: 23.

### 2. Fab-Knob-CEA+Hole (102)

As shown in FIG. 1B (102) and 1×(307-312), a multispecific antibody (e.g., bispecific antibody) can be prepared, which includes (a) a first polypeptide including, preferably from N-terminus to C-terminus: a VH, a CH1 domain, optionally a first hinge region, a first Fc region (e.g., CH2 domain and CH3 domain), optionally a linker peptide, and a single-domain antibody variable domain (VHH); (b) a second polypeptide including, preferably from N-terminus to C-terminus: optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 6. In some embodiments, the second polypeptide comprises a sequence that is at least 80%, 85%, 90%, 95%, 96%, 97%, 98%, 99%, or 100% identical to SEQ ID NO: 8. In some embodiments, the third polypeptide comprises a sequence that is at least 80%, 85%, 90%, 95%, 96%, 97%, 98%, 99%, or 100% identical to SEQ ID NO: 23.

### 3. Fab-Knob+Hole-CEA (103)

As shown in FIG. 1C, the multispecific antibody (e.g., bispecific antibody) can be prepared, which includes (a) a first polypeptide including, preferably from N-terminus to C-terminus: a VH, a CH1 domain, optionally a first hinge region, and a first Fc region (e.g., CH2 domain and CH3 domain); (b) a second polypeptide including, preferably from N-terminus to C-terminus: optionally a second hinge region, a second Fc region (e.g., CH2 domain and CH3 domain), optionally a linker peptide, and a single-domain antibody variable domain (VHH); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 2. In some embodiments, the second polypeptide comprises a sequence that is at least 80%, 85%, 90%, 95%, 96%, 97%, 98%, 99%, or 100% identical to SEQ ID NO: 12. In some embodiments, the third polypeptide comprises a sequence that is at least 80%, 85%, 90%, 95%, 96%, 97%, 98%, 99%, or 100% identical to SEQ ID NO: 23.

### 4. CEA-Knob+Fab-Hole (104)

As shown in FIG. 1D, the multispecific antibody (e.g., bispecific antibody) can be prepared, which includes (a) a first polypeptide including, preferably from N-terminus to C-terminus: a single-domain antibody variable domain (VHH), optionally a first hinge region, and a first Fc region (e.g., CH2 domain and CH3 domain); (b) a second polypeptide including, preferably from N-terminus to C-terminus: a VH, a CH1 domain, optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19.

In some embodiments, the first polypeptide comprises a sequence that is at least 80%, 85%, 90%, 95%, 96%, 97%, 98%, 99%, or 100% identical to SEQ ID NO: 4. In some embodiments, the second polypeptide comprises a sequence that is at least 80%, 85%, 90%, 95%, 96%, 97%, 98%, 99%, or 100% identical to SEQ ID NO: 9. In some embodiments, the third polypeptide comprises a sequence that is at least 80%, 85%, 90%, 95%, 96%, 97%, 98%, 99%, or 100% identical to SEQ ID NO: 23.

### 5. Knob-CEA+Fab-Hole (105)

As shown in FIG. 1E, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a linker peptide, and a single-domain antibody variable domain (VHH); (b) a second polypeptide including, preferably from N-terminus to C-terminus: a VH, a CH1 domain, optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 5. In some embodiments, the second polypeptide comprises a sequence that is at least 80%, 85%, 90%, 95%, 96%, 97%, 98%, 99%, or 100% identical to SEQ ID NO: 9. In some embodiments, the third polypeptide comprises a sequence that is at least 80%, 85%, 90%, 95%, 96%, 97%, 98%, 99%, or 100% identical to SEQ ID NO: 23.

### 6. Knob+Fab-Hole-CEA (106)

As shown in FIG. 1F, the multispecific antibody (e.g., bispecific antibody) can be prepared, which includes (a) a first polypeptide including, preferably from N-terminus to C-terminus: optionally a first hinge region, and a first Fc region (e.g., CH2 domain and CH3 domain); (b) a second polypeptide including, preferably from N-terminus to C-terminus: a VH, a CH1 domain, optionally a second hinge region, a second Fc region (e.g., CH2 domain and CH3 domain), optionally a linker peptide, and a single-domain antibody variable domain (VHH); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 13. In some embodiments, the third polypeptide comprises a sequence that is at least 80%, 85%, 90%, 95%, 96%, 97%, 98%, 99%, or 100% identical to SEQ ID NO: 23.

### 7. CEA-Knob-Fab-+Hole (107)

As shown in FIG. 1G, the multispecific antibody (e.g., bispecific antibody) can be prepared, which includes (a) a first polypeptide including, preferably from N-terminus to C-terminus: a single-domain antibody variable domain (VHH), optionally a first hinge region, a first Fc region (e.g., CH2 domain and CH3 domain), optionally a linker peptide, a VH, and a CH1 domain; (b) a second polypeptide including, preferably from N-terminus to C-terminus: optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 7. In some embodiments, the second polypeptide comprises a sequence that is at least 80%, 85%, 90%, 95%, 96%, 97%, 98%, 99%, or 100% identical to SEQ ID NO: 8. In some embodiments, the third polypeptide comprises a sequence that is at least 80%, 85%, 90%, 95%, 96%, 97%, 98%, 99%, or 100% identical to SEQ ID NO: 23.

### 8. Knob-Fab+CEA-Hole (108)

As shown in FIG. 1H, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a linker peptide, a VH, and a CH1 domain; (b) a second polypeptide including, preferably from N-terminus to C-terminus: a single-domain antibody variable domain (VHH), optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 3. In some embodiments, the second polypeptide comprises a sequence that is at least 80%, 85%, 90%, 95%, 96%, 97%, 98%, 99%, or 100% identical to SEQ ID NO: 11. In some embodiments, the third polypeptide comprises a sequence that is at least 80%, 85%, 90%, 95%, 96%, 97%, 98%, 99%, or 100% identical to SEQ ID NO: 23.

### 9. Knob-Fab+Hole-CEA (109)

As shown in FIG. 1I, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a first linker peptide, a VH, and a CH1 domain; (b) a second polypeptide including, preferably from N-terminus to C-terminus: optionally a second hinge region, a second Fc region (e.g., CH2 domain and CH3 domain), optionally a second linker peptide, and a single-domain antibody variable domain (VHH); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 3. In some embodiments, the second polypeptide comprises a sequence that is at least 80%, 85%, 90%, 95%, 96%, 97%, 98%, 99%, or 100% identical to SEQ ID NO: 12. In some embodiments, the third polypeptide comprises a sequence that is at least 80%, 85%, 90%, 95%, 96%, 97%, 98%, 99%, or 100% identical to SEQ ID NO: 23.

### 10. CEA-Knob+Hole-Fab (110)

As shown in FIG. 1J, the multispecific antibody (e.g., bispecific antibody) can be prepared, which includes (a) a first polypeptide including, preferably from N-terminus to C-terminus: a single-domain antibody variable domain (VHH), optionally a first hinge region, and a first Fc region (e.g., CH2 domain and CH3 domain); (b) a second polypeptide including, preferably from N-terminus to C-terminus: optionally a second hinge region, a second Fc region (e.g., CH2 domain and CH3 domain), optionally a linker peptide, a VH, and a CH1 domain; and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 4. In some embodiments, the second polypeptide comprises a sequence that is at least 80%, 85%, 90%, 95%, 96%, 97%, 98%, 99%, or 100% identical to SEQ ID NO: 10. In some embodiments, the third polypeptide comprises a sequence that is at least 80%, 85%, 90%, 95%, 96%, 97%, 98%, 99%, or 100% identical to SEQ ID NO: 23.

### 11. Knob+CEA-Hole-Fab (111)

As shown in FIG. 1K, the multispecific antibody (e.g., bispecific antibody) can be prepared, which includes (a) a first polypeptide including, preferably from N-terminus to C-terminus: optionally a first hinge region, and a first Fc region (e.g., CH2 domain and CH3 domain); (b) a second polypeptide including, preferably from N-terminus to C-terminus: a single-domain antibody variable domain (VHH), optionally a second hinge region, a second Fc region (e.g., CH2 domain and CH3 domain), optionally a linker peptide, a VH, and a CH1 domain; and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 14. In some embodiments, the third polypeptide comprises a sequence that is at least 80%, 85%, 90%, 95%, 96%, 97%, 98%, 99%, or 100% identical to SEQ ID NO: 23.

### 12. Knob-CEA+Hole-Fab (112)

As shown in FIG. 1L, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a first linker peptide, and a single-domain antibody variable domain (VHH); and (b) a second polypeptide including, preferably from N-terminus to C-terminus: optionally a second hinge region, a second Fc region (e.g., CH2 domain and CH3 domain), optionally a second linker peptide, a VH, and a CH1 domain; and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 5. In some embodiments, the second polypeptide comprises a sequence that is at least 80%, 85%, 90%, 95%, 96%, 97%, 98%, 99%, or 100% identical to SEQ ID NO: 10. In some embodiments, the third polypeptide comprises a sequence that is at least 80%, 85%, 90%, 95%, 96%, 97%, 98%, 99%, or 100% identical to SEQ ID NO: 23.

### 13. CEA-Fab-Knob-+Hole (201)

As shown in FIG. 1M, the multispecific antibody (e.g., bispecific antibody) can be prepared, which includes (a) a first polypeptide including, preferably from N-terminus to C-terminus: a VH, a CH1 domain, optionally a first hinge region, a first Fc region (e.g., CH2 domain and CH3 domain); (b) a second polypeptide including, preferably from N-terminus to C-terminus: optionally a second hinge region, and a second Fc region (e.g., CH2 domain and CH3 domain); and (c) a third polypeptide including, preferably from N-terminus to C-terminus: a VL, a CL, optionally a third linker peptide, and a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen.

In some embodiment, the single-domain antibody variable domain (VHH) is linked to the light chain constant region (CL) of the Fab.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 2. In some embodiments, the second polypeptide comprises a sequence that is at least 80%, 85%, 90%, 95%, 96%, 97%, 98%, 99%, or 100% identical to SEQ ID NO: 8. In some embodiments, the third polypeptide comprises a sequence that is at least 80%, 85%, 90%, 95%, 96%, 97%, 98%, 99%, or 100% identical to SEQ ID NO: 39.

### 14. Knob+CEA-Fab-Hole (202)

As shown in FIG. 1N, the multispecific antibody (e.g., bispecific antibody) can be prepared, which includes (a) a first polypeptide including, preferably from N-terminus to C-terminus: optionally a first hinge region, and a first Fc region (e.g., CH2 domain and CH3 domain); (b) a second polypeptide including, preferably from N-terminus to C-terminus: a VH, a CH1 domain, optionally a second hinge region, and a second Fc region (e.g., CH2 domain and CH3 domain); and (c) a third polypeptide including, preferably from N-terminus to C-terminus: a VL, a CL, optionally a linker peptide, a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen.

In some embodiment, the single-domain antibody variable domain (VHH) is linked to the light chain constant region (CL) of the Fab.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 9. In some embodiments, the third polypeptide comprises a sequence that is at least 80%, 85%, 90%, 95%, 96%, 97%, 98%, 99%, or 100% identical to SEQ ID NO: 39.

### 15. CEA-Fab-Knob-+Hole (203)

As shown in FIG. 1O, the multispecific antibody (e.g., bispecific antibody) can be prepared, which includes (a) a first polypeptide comprising from N-terminus to C-terminus: a VH, a CH1 domain, optionally a first hinge region, a first CH2 domain, and a first CH3 domain; (b) a second polypeptide comprising from N-terminus to C-terminus: optionally a second hinge region, a second CH2 domain, and a second CH3 domain; and (c) a third polypeptide comprising from N-terminus to C-terminus: a single-domain antibody variable domain (VHH), optionally a third linker peptide, a VL, and a CL domain, In some embodiments, the VH and the VL associate with each other, forming an antigen binding site of a Fab that specifically binds to a T cell antigen. In some embodiments, the VHH specifically binds to a tumor-associated antigen.

In some embodiment, the single-domain antibody variable domain (VHH) is linked to the light chain variable region of the Fab.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 2. In some embodiments, the second polypeptide comprises a sequence that is at least 80%, 85%, 90%, 95%, 96%, 97%, 98%, 99%, or 100% identical to SEQ ID NO: 8. In some embodiments, the third polypeptide comprises a sequence that is at least 80%, 85%, 90%, 95%, 96%, 97%, 98%, 99%, or 100% identical to SEQ ID NO: 40.

### 16. Knob+CEA-Fab-Hole (204)

As shown in FIG. 1P, the multispecific antibody (e.g., bispecific antibody) can be prepared, which includes (a) a first polypeptide comprising from N-terminus to C-terminus: optionally a first hinge region, a first CH2 domain, and a first CH3 domain; (b) a second polypeptide comprising from N-terminus to C-terminus: a VH, a CH1 domain, optionally a second hinge region, a second CH2 domain, and a second CH3 domain; and (c) a third polypeptide comprising from N-terminus to C-terminus: a single-domain antibody variable domain (VHH), optionally a linker peptide, a VL, and a CL domain. In some embodiments, the VH and the VL associate with each other, forming an antigen binding site of a Fab that specifically binds to a T cell antigen. In some embodiments, the VHH specifically binds to a tumor-associated antigen.

In some embodiment, the single-domain antibody variable domain (VHH) is linked to the light chain variable region of the Fab.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 9. In some embodiments, the third polypeptide comprises a sequence that is at least 80%, 85%, 90%, 95%, 96%, 97%, 98%, 99%, or 100% identical to SEQ ID NO: 40.

### 17. CEA-Fab-Knob-+Hole (205)

As shown in FIG. 1Q, the multispecific antibody (e.g., bispecific antibody) can be prepared, which includes (a) a first polypeptide including, preferably from N-terminus to C-terminus: a single-domain antibody variable domain (VHH), optionally a first linker peptide, a VH, a CH1 domain, optionally a first hinge region, and a first Fc region (e.g., CH2 domain and CH3 domain); (b) a second polypeptide including, preferably from N-terminus to C-terminus: optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiment, the single-domain antibody variable domain (VHH) is linked to the heavy chain variable region of the Fab.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 41. In some embodiments, the second polypeptide comprises a sequence that is at least 80%, 85%, 90%, 95%, 96%, 97%, 98%, 99%, or 100% identical to SEQ ID NO: 8. In some embodiments, the third polypeptide comprises a sequence that is at least 80%, 85%, 90%, 95%, 96%, 97%, 98%, 99%, or 100% identical to SEQ ID NO: 23.

### 18. Knob+CEA-Fab-Hole (206)

As shown in FIG. 1R, the multispecific antibody (e.g., bispecific antibody) can be prepared, which includes (a) a first polypeptide including, preferably from N-terminus to C-terminus: optionally a first hinge region, and a first Fc region (e.g., CH2 domain and CH3 domain); (b) a second polypeptide including, preferably from N-terminus to C-terminus: a single-domain antibody variable domain (VHH), optionally a second linker peptide, a VH, a CH1 domain, optionally a second hinge region, and a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiment, the single-domain antibody variable domain (VHH) is linked to the heavy chain variable region of the Fab.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 42. In some embodiments, the third polypeptide comprises a sequence that is at least 80%, 85%, 90%, 95%, 96%, 97%, 98%, 99%, or 100% identical to SEQ ID NO: 23.

### 19. Fab (Heavy)-Knob-CEA+Fab (Light)-Hole (207)

As shown in FIG. 1S, the multispecific antibody (e.g., bispecific antibody) can be prepared, which includes (a) a first polypeptide including, preferably from N-terminus to C-terminus: a VH, a CH1 domain, optionally a first hinge region, a first Fc region (e.g., CH2 domain and CH3 domain), optionally a first linker peptide, and a single-domain antibody variable domain (VHH); and (b) a second polypeptide including, preferably from N-terminus to C-terminus: a VL, a CL, optionally a second hinge region, and a second Fc region (e.g., CH2 domain and CH3 domain). In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 6. In some embodiments, the second polypeptide comprises a sequence that is at least 80%, 85%, 90%, 95%, 96%, 97%, 98%, 99%, or 100% identical to SEQ ID NO: 44.

### 20. Fab (Heavy)-Knob+Fab (Light)-Hole-CEA (208)

As shown in FIG. 1T, the multispecific antibody (e.g., bispecific antibody) can be prepared, which includes (a) a first polypeptide including, preferably from N-terminus to C-terminus: a VH, a CH1 domain, optionally a first hinge region, and a first Fc region (e.g., CH2 domain and CH3 domain); and (b) a second polypeptide including, preferably from N-terminus to C-terminus: a VL, a CL, optionally a second hinge region, a second Fc region (e.g., CH2 domain and CH3 domain), optionally a second linker peptide, and a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 2. In some embodiments, the second polypeptide comprises a sequence that is at least 80%, 85%, 90%, 95%, 96%, 97%, 98%, 99%, or 100% identical to SEQ ID NO: 46.

### 21. Fab (Light)-Knob-CEA+Fab (Heavy)-Hole (209)

As shown in FIG. 1U, the multispecific antibody (e.g., bispecific antibody) can be prepared, which includes (a) a first polypeptide including, preferably from N-terminus to C-terminus: a VL, a CL, optionally a first hinge region, a first Fc region (e.g., CH2 domain and CH3 domain), optionally a first linker peptide, and a single-domain antibody variable domain (VHH); and (b) a second polypeptide including, preferably from N-terminus to C-terminus: a VH, a CH1 domain, optionally a second hinge region, and a second Fc region (e.g., CH2 domain and CH3 domain). In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 45. In some embodiments, the second polypeptide comprises a sequence that is at least 80%, 85%, 90%, 95%, 96%, 97%, 98%, 99%, or 100% identical to SEQ ID NO: 9.

### 22. Fab (Light)-Knob+Fab (Heavy)-Hole-CEA (210)

As shown in FIG. 1V, the multispecific antibody (e.g., bispecific antibody) can be prepared, which includes (a) a first polypeptide including, preferably from N-terminus to C-terminus: a VL, a CL, optionally a first hinge region, and a first Fc region (e.g., CH2 domain and CH3 domain); and (b) a second polypeptide including, preferably from N-terminus to C-terminus: a VH, a CH1 domain, optionally a second hinge region, and a second Fc region (e.g., CH2 domain and CH3 domain), optionally a second linker peptide, and a single-domain antibody variable domain (VHH). In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1 or 20. In some embodiments, the second polypeptide comprises a sequence that is at least 80%, 85%, 90%, 95%, 96%, 97%, 98%, 99%, or 100% identical to SEQ ID NO: 8 or 21.

In some embodiments, the Fab can target CD3 (e.g., human CD3). In some embodiments, the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 43. In some embodiments, the second polypeptide comprises a sequence that is at least 80%, 85%, 90%, 95%, 96%, 97%, 98%, 99%, or 100% identical to SEQ ID NO: 13.

### Antigen-Binding Protein Construct Characteristics

The anti-CD3, anti-TAA (tumor associated antigen), or anti-CD3/TAA antigen-binding protein construct (e.g., antibodies, bispecific antibodies, trispecific antibodies, multispecific antibodies, or antibody fragments thereof), can include an antigen binding site that is derived from any anti-CD3 antibody, anti-TAA antibody (e.g., anti-CEACAM5, anti-EGFR or anti-HER2), or any antigen-binding fragment thereof as described herein.

In some embodiments, the antibodies or antigen-binding fragments thereof described herein are CEACAM5 antagonist. In some embodiments, the antibodies or antigen-binding fragments thereof are CEACAM5 agonist. In some embodiments, the antibodies or antigen-binding fragments thereof as described herein are CD3 antagonist. In some embodiments, the antibodies or antigen-binding fragments thereof are CD3 agonist. In some embodiments, the antibodies or antigen-binding fragments thereof as described herein are EGFR antagonist. In some embodiments, the antibodies or antigen-binding fragments thereof are EGFR agonist. In some embodiments, the antibodies or antigen-binding fragments thereof as described herein are HER2 antagonist. In some embodiments, the antibodies or antigen-binding fragments thereof are HER2 agonist.

In some embodiments, the antibodies, or antigen-binding fragments thereof described herein can bind to CD3 and a target tumor associated antigen (e.g., CEACAM5, EGFR or HER2), thereby bridging T cells and target cells; activating T cells; and inducing directly killing the cancer cells by the T cells.

In some embodiments, the antibody (or antigen-binding fragments thereof) specifically binds to the target antigen (e.g., CD3, CEACAM5, EGFR or HER2) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (Kd=koff/kon). In some embodiments, Kd is less than 1×10⁻⁴ M, less than 1×10⁻⁵ M, less than 1×10⁻⁶ M, less than 1×10⁻⁷M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, Kd is greater than 1×10⁻⁴ M, greater than 1×10⁻⁵ M, greater than 1×10⁻⁶ M, greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. Furthermore, Ka can be deduced from Kd by the formula Ka=1/Kd.

General techniques for measuring the affinity of an antibody for an antigen include, e.g., ELISA, RIA, and surface plasmon resonance (SPR).

In some embodiments, the expression level of the antibodies, the antigen binding fragments thereof, or the antigen-binding protein constructs described herein is at least or about 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 50, 100, 200, 300, 400, 500, 600, 700, 800, 900, 1000, 2000, 3000, 4000, 5000, 6000, 7000, 8000, 9000, 10000, 20000, 50000, or 100000 mg/L, as determined using the method described herein. In some embodiments, the percentage of multispecific antibody (e.g., bispecific antibody) formed, as determined by size-exclusion chromatography as described herein, is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 93%, at least 94%, at least 95%, at least 96%, or at least 97% of the total protein level.

In some embodiments, the antibodies, the antigen binding fragments thereof, or the antigen-binding protein constructs described herein have a cell killing EC50 of less than or about 10 pM, less than or about 9 pM, less than or about 8 pM, less than or about 7 pM, less than or about 6 pM, less than or about 5 pM, less than or about 4 pM, less than or about 3 pM, less than or about 2 pM, less than or about 1 pM, less than or about 0.8 pM, less than or about 0.5 pM, less than or about 0.3 pM, less than or about 0.2 pM, less than or about 0.1 pM, as determined using the method described herein. In some embodiments, the antibodies, antigen binding fragments thereof, or the antigen-binding protein constructs described herein have a cell killing EC50 value that is about 0.1 pM to about 10 pM, about 1 pM to 10 pM, about 5 pM to 10 pM, about 0.1 pM to 5 pM, or about 0.1 pM to 1 pM. In some embodiments, the antibodies, the antigen binding fragments thereof, or the antigen-binding protein constructs described herein have a cell killing EC50 value that is less than or about 50%, less than or about 30%, less than or about 20%, less than or about 10%, less than or about 5%, less than or about 1% as compared to that of an isotype control antibody.

In some embodiments, the antibodies, the antigen binding fragments thereof, the antigen-binding protein constructs, or protein complexes described herein have a cell-binding EC50 of less than or about 0.1 nm, 0.25 nM, 0.5 nM, 0.75 nM, 1 nM, 1.25 nM, 1.5 nM, 2 nM, 2.5 nM, 5 nM, 7.5 nM, 10 nM, or 20 nM, as determined using the methods described herein.

In some embodiments, the antibodies, the antigen binding fragments thereof, the antigen-binding protein constructs, or protein complexes described herein have a Tm value of higher than 60° C., 60.5° C., 61° C., 61.5° C., 62° C., 62.5° C., 63° C., 63.5° C., 64° C., 65° C., 66° C., 67° C., 68° C., 69° C., 70° C., 71° C., 72° C., 73° C. or 74° C., as determined using the methods described herein.

In some embodiments, the antibodies, the antigen binding fragments thereof, the antigen-binding protein constructs, or protein complexes described herein have a peak absorbance at less than 560 nm, 555 nm, 550 nm, 545 nm, 540 nm, 535 nm, or 530 nm in 300 mM, 400 mM, 500 mM, 600 mM, 700 mM, 800 mM, 900 mM or 1000 mM ammonium sulfate as measured by salt-gradient affinity capture self-interaction nanoparticle spectroscopy (SGAC-SINS). In some embodiments, the antibodies, the antigen binding fragments thereof, the antigen-binding protein constructs, or protein complexes described herein have a hydrophobicity that is similar to Ofatumumab as measured by salt-gradient affinity capture self-interaction nanoparticle spectroscopy (SGAC-SINS).

In some embodiments, the antibodies, the antigen binding fragments thereof, the antigen-binding protein constructs, or protein complex described herein have a tumor growth inhibition percentage (TGI_{TV}%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI_{TV}% can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI_{TV}%) is calculated using the following formula: TGI_{TV} (%)=[1−(Ti−T0)/(Vi−V0)]×100 (Ti is the average tumor volume in the treatment group on day i. T0 is the average tumor volume in the treatment group on day zero. Vi is the average tumor volume in the control group on day i. V0 is the average tumor volume in the control group on day zero).

In some embodiments, the antibodies, the antigen binding fragments thereof, the antigen-binding protein constructs, or protein complex described herein have a serum stability of more than 50%, 55%, 60%, 65% 70%, 75%, 80%, 85%, 90%, 91%, 92%, 93%, 94%, 95%, 96%, 97%, 98% or 99% cell binding after 1-10 days of storage in human serum, as determined using the methods described herein.

In some embodiments, the antibodies, the antigen binding fragments thereof, or the antigen-binding protein constructs have a functional Fc region. In some embodiments, effector function of a functional Fc region is antibody-dependent cell-mediated cytotoxicity (ADCC) and/or complement-dependent cytotoxicity (CDC). In some embodiments, the Fc region is human IgG1, human IgG2, human IgG3, or human IgG4.

In some embodiments, the antibodies, the antigen binding fragments thereof, or the antigen-binding protein constructs do not have a functional Fc region. For example, the antibodies or antigen binding fragments are Fab, Fab′, F(ab′)₂, and Fv fragments. In some embodiments, the antibodies, antigen binding fragments, or the antigen-binding protein constructs have a Fc region that includes one or more mutations to reduce the effector function. In some embodiments, the antibodies, the antigen binding fragments thereof, or the antigen-binding protein constructs described herein do not have antibody-dependent cell-mediated cytotoxicity (ADCC) or complement dependent cytotoxicity (CDC).

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the multi-specific antibody including the bispecific antibody described herein (e.g., CD3/CEACAM5 bispecific antibody) has an asymmetric structure comprising: 2, 3, 4, 5, or 6 antigen binding sites. In some embodiments, the multispecific antibody described herein comprises 2, 3, 4, 5, or 6 antigen binding sites (e.g., antigen binding scFv domains, Fab, or VHH) that target CEACAM5. In some embodiments, the CEACAM5 binding Fab domain comprises the same variable domain sequence. In some embodiments, the CEACAM5 binding Fab domain comprises different variable domain sequences.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68 (1-3):9-16 (October 1997); Morea et al., J Mol Biol. 275 (2):269-94 (January 0.1998); Chothia et al., Nature 342 (6252):877-83 (December 1989); Ponomarenko and Bourne, BMC Structural Biology 7:64 (2007); Kontermann, R., & Dübel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody or antigen-binding protein can include an intact immunoglobulin molecule (e.g., IgG1, IgG2a, IgG2b, IgG3, IgM, IgD, IgE, IgA) or fragments thereof. The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody or antigen-binding protein can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen-binding domain” or “antigen-binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antibodies or antigen-binding fragments thereof can bind to two different antigens or two different epitopes. In some embodiments, the antibodies or antigen-binding fragments thereof can bind to three different antigens or three different epitopes.

The Fab fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

In some embodiments, the Fc region can be further modified to increase or decrease effector functions as well as serum half-life.

Any of the antibodies, antigen-binding fragments thereof, or antigen-binding proteins described herein can be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of the antibodies, antigen-binding fragments thereof, or antigen-binding proteins in vitro (e.g., in tissue culture or when stored as a pharmaceutical composition) or in vivo (e.g., in a human).

In some embodiments, the antibodies, antigen-binding fragments thereof, or antigen-binding proteins (e.g., multispecific antibodies) described herein can be conjugated to a therapeutic agent. The antibody-drug conjugate comprising the antibodies, antigen-binding fragments thereof, or antigen-binding proteins can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multispecific antibody or antigen-binding fragment thereof described herein (e.g., CD3/CEACAM5 multispecific antibody) binds to CD3 (e.g., human CD3) with a binding affinity that is about 50%, about 60%, about 70%, about 80%, about 90%, about 100%, about 110%, about 120%, about 130%, about 140%, about 150%, or about 200% to that of an antibody (e.g., an anti-CD3 antibody) comprising the same antigen binding region (e.g., Fab, scFv or VHH) of the multi-specific antibody.

In some embodiments, the multispecific antibody or antigen-binding fragment thereof described herein (e.g., CD3/CEACAM5 multispecific antibody) binds to CEACAM5 with a binding affinity that is about 50%, about 60%, about 70%, about 80%, about 90%, about 100%, about 110%, about 120%, about 130%, about 140%, about 150%, or about 200% to that of an antibody (e.g., an anti-CEACAM5 antibody) comprising the same antigen binding region (e.g., Fab, scFv or VHH) of the multi-specific antibody.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof or the antigen-binding protein constructs by recombinant techniques.

As used herein, a “vector” is any construct capable of delivering one or more polynucleotide (s) of interest to a host cell when the vector is introduced to the host cell. An “expression vector” is capable of delivering and expressing the one or more polynucleotide (s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HEK293 cells; and plant cells. Appropriate culture mediums and conditions for the host cells described herein are known in the art.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, ³⁰, 35%, 4%0%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antibodies or Antigen-Binding Protein Constructs

An isolated fragment of human protein (e.g., CD3 or CEACAM5) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R.

Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding affinities.

Variants of the antibodies, antigen-binding fragments, or the antigen-binding protein constructs described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen (s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain (s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions T366W. The other heavy chain can have one or more the following substitutions T366S, L368A, and Y407V. Furthermore, a substitution (-ppcpScp-->-ppcpPcp-) can also be introduced at the hinge regions of both substituted IgG.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

### Methods of Treatment

The methods described herein include methods for the treatment of disorders associated with cancer. Generally, the methods include administering a therapeutically effective amount of engineered multispecific antibodies (e.g., bispecific antibodies) or the antigen-binding protein constructs as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., antigen-binding protein constructs) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, antigen-binding protein constructs, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing CEACAM5.

In some embodiments, the cancers are lung cancers, colorectal cancer, head and neck cancer, stomach cancer, pancreatic cancer, urothelial cancer, breast cancer, cervical cancer, or endometrial cancer.

In some embodiments, the cancer cells described herein is cell lines, e.g., H1395 cells. In some embodiments, the cancer cells have an elevated CEACAM5 level, e.g., at least 10%, at least 20%, at least 30%, at least 40%, at least 50% higher than non-cancerous cells.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antigen-binding protein constructs, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, an antigen-binding protein construct, or an antibody-drug conjugate is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the agent used.

Effective amounts and schedules for administering the antibodies, antigen-binding protein constructs, antibody-encoding polynucleotides, antibody-drug conjugates, and/or compositions disclosed herein may be determined empirically, and making such determinations is within the skill in the art.

A typical dosage of an effective amount of an antibody or antigen-binding protein construct is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antigen-binding protein constructs, antibody-drug conjugates, or pharmaceutical composition (e.g., any of the antibodies, antigen-binding fragments, antigen-binding protein constructs, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody, antigen-binding fragment thereof, or the antigen-binding protein construct can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antigen-binding protein constructs, antigen binding proteins, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies, antigen-binding fragments thereof, or antigen-binding protein constructs (e.g., any of the antibodies, antibody fragments, or antigen-binding protein constructs described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies, antigen-binding fragments, or antigen-binding protein constructs described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, antigen-binding protein constructs, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody, antibody fragment, or antigen-binding protein constructs in vivo.

The pharmaceutical compositions can be included in a container, pack, or dispenser together with instructions for administration. The disclosure also provides methods of manufacturing the antibodies, antigen binding fragments thereof, or antigen-binding protein constructs for various uses as described herein.

### EXAMPLES

The invention is further described in the following examples, which do not limit the scope of the invention described in the claims.

### Materials and Methods

### Preparation of Human Peripheral Blood Mononuclear Cells (PBMC)

PBMCs were prepared by density gradient centrifugation, and blood samples were from blood banks or healthy human donors.

The blood of healthy donors was stored in an EDTA-containing anticoagulation tube, and allowed to stand for 10 minutes. An equal volume of 2% PBS (pH 7.4) was added and mix thoroughly. 15 ml Ficoll Paque PLUS low-density centrifugal fluid (GE) was added into a 50 ml Falcon tube, and 30 ml of diluted fresh blood was aspirated and slowly added to the upper layer of the density gradient solution along the side wall of the Falcon tube. The centrifuge was adjusted to the brake off state, the test tube was loaded on the centrifuge, and centrifuged at 1450 rpm at room temperature for 45 minutes. The test tube was gently taken out and visually observed to make sure that the blood in the centrifuge tube was divided into three layers (the upper layer is the serum layer, the white part of the middle layer is the peripheral blood mononuclear cells, and the bottom layer is the red blood cells). The upper yellow and transparent serum layer was carefully aspirated, leaving only the middle layer, which was transferred to a new 15 ml centrifuge tube. An equal volume of the gradient solution was added and the diluted middle layer was centrifuged at 1450 rpm for 30 minutes at room temperature. The upper suspension was removed, retaining the bottom cell pellet. The cells were resuspended and washed 3 times until the upper layer was clear. 1 ml of 2% FBS-PBS buffer was added to resuspend the cells. After the PBMC was counted, the cells were resuspended in the RPMI1640 medium containing 10% FBS (GIBCO) and 1% L-glutamine (GIBCO) and cultured in a CO₂ incubator at 37° C. and 5% CO₂.

### Target Cells

To evaluate CEA-targeting bispecific antibody molecules, the following tumor cell lines were used: human lung adenocarcinoma cell line NCI-H1573, which expresses high levels of CEA; human primary pancreatic adenocarcinoma cell line BxPC-3, which expresses a medium level of CEA; human lung adenocarcinoma cell NCI-H1395, which expresses a medium level of CEA; human colon cancer cell HT-29, which expresses a medium level of CEA; human non-small cell lung cancer cell line NCI-H1650, which expresses low levels of human CEA; human non-small cell lung adenocarcinoma cell NCI-H157, which does not express CEA.

In addition, the human T-cell leukemia cell line Jurkat (Clone E6-1, Chinese Academy of Sciences in Shanghai, Cell Bank, SCSP-513) and the human lung adenocarcinoma cell line NCI-H1573 were used to evaluate the binding of different bispecific constructs to the human CD3 and human CEA on these cells.

### Example 1. Preparation of the Anti-CD3 Antibodies and Anti-CEA Antibodies, and Bispecific Antibodies

On the basis of a murine anti-CD3 antibody, a humanized CD3 antibody TA1 was obtained through mutation, library construction, humanization modification and screening. For comparison purpose, 6 anti-CD3 antibodies were used and were named TA2, TA3, TA4, TA5, TA6, TA7, respectively.

CEA antibodies were obtained by immunizing llamas (lama). 250 μg of recombinant tumor antigen human CEA (CEACAM-5/CD66e, ACRO systems, CE5-H5226) was subcutaneously injected to immunize adult alpaca, once every other month, for a total of 5 immunizations. PBMC cells were isolated and RNA was extracted from these PBMC cells to construct a phage antibody library. After the library was packaged to form phage particles, it was panned using the liquid phase method. Specifically, the phage was combined with the biotinylated CEA antigen solution, and then separated by streptavidin magnetic beads. After screening using ELISA and CEA-overexpressing engineered cells, the anti-CEA antibody C17 was selected for further experiments. It was used in making antibodies as shown in the structural diagrams in FIGS. 1A-1X.

Bispecific antibody structure design: bispecific antibodies with 22 different structures were designed (FIGS. 1A-1V). When the CD3 Fab or CEA VHH is located at the C-terminus of Fc, GGGGSGGGGS is used as a linker to connect the two fragments. When the CD3 Fab or CEA VHH is located at the N-terminus of Fc, the Fc hinge region (Hinge) is used as a linker to connect the two fragments.

The structures of the CD3-CEA bispecific antibodies in some embodiments of the present disclosure are shown in FIGS. 1A-1X. The asymmetric structure design of two chains containing Fc has one CEA antigen binding domain and one CD3 antigen binding domain. The CEA binding domain is in the form of single domain antibody (VHH) and the CD3 binding domain is in the form of a Fab. The Fc region endows the bispecific antibodies with a longer half-life and good stability. The Knob into Hole (KIH) design of the two-chain greatly reduces mismatches, improves the homogeneity of the protein and the yield of the bispecific antibodies. The specific format of the bispecific antibodies is shown in FIGS. 1A-1X.

In addition, FIG. 1W shows the structures of multiple bispecific antibodies with different CD3 binding domains (TA2-TA7). Here, six bispecific antibodies with the configuration in FIG. 1W are named 301 (TA2), 302 (TA3), 303 (TA4), 304 (TA5), 305 (TA6) and 306 (TA7), respectively.

FIG. 1X shows the structures of multiple bispecific antibodies with different CD3 binding domains (TA2-TA7). Here, six bispecific antibodies with the configuration in FIG. 1X are named 307 (TA2), 308 (TA3), 309 (TA4), 310 (TA5), 311 (TA6) and 312 (TA7), respectively.

The two heavy chains IgG4 Fe are knob-Fe and hole-Fe respectively, and the sequences of knob-Fe and hole-Fe are preferably those shown in the table below; the light chain is a kappa light chain.

TABLE 1
Fc sequence list

| | SEQ | |
|---|---|---|
| | ID | |
| Fc | NO. | Sequence |
| knob-Fc | 1 | ESKYGPPCPPCPAPEAAGGPSVFLF |
| | | PPKPKDTLMISRTPEVTCVVVDVSQ |
| | | EDPEVQFNWYVDGVEVHNAKTKPRE |
| | | EQFNSTYRVVSVLTVLHQDWLNGKE |
| | | YKCKVSNKGLPSSIEKTISKAKGQP |
| | | REPQVYTLPPCQEEMTKNQVSLWCL |
| | | VKGFYPSDIAVEWESNGQPENNYKT |
| | | TPPVLDSDGSFFLYSRLTVDKSRWQ |
| | | EGNVFSCSVMHEALHNHYTQKSLSL |
| | | SL |
| hole-Fc | 8 | ESKYGPPCPPCPAPEAAGGPSVFLF |
| | | PPKPKDTLMISRTPEVTCVVVDVSQ |
| | | EDPEVQFNWYVDGVEVHNAKTKPRE |
| | | EQFNSTYRVVSVLTVLHQDWLNGKE |
| | | YKCKVSNKGLPSSIEKTISKAKGQP |
| | | REPQVCTLPPSQEEMTKNQVSLSCA |
| | | VKGFYPSDIAVEWESNGQPENNYKT |
| | | TPPVLDSDGSFFLVSRLTVDKSRWQ |
| | | EGNVFSCSVMHEALHNRFTQKSLSL |
| | | SL |

CD3-targeting antibody (TA1, TA2, TA3, TA4, TA5, TA6, TA7), and CEA-targeting single domain antibody C17 heavy chain sequences are preferably those shown those shown in the table below.

TABLE 2
Sequence of antibody heavy chain

| Anti- | | |
|---|---|---|
| body | SEQ | |
| Heavy | ID | |
| Chain | NO. | Sequence |
| TA1 VH | 25 | EVKLLESGGGLVQPKGSLKLSCAAS |
| | | GFTFNTYAMNWVRQAPGKGLEWVAR |
| | | IRSKYNNYATYYADSVKDRFTISRD |
| | | DSQSILYLQMNNLKTEDTAMYYCVR |
| | | HGNFGNSYVSWFAYWGQGTLVTVSS |
| TA2 VH | 26 | QVQLVQSGGGVVQPGRSLRLSCKAS |
| | | GYTFTRYTMHWVRQAPGKGLEWIGY |
| | | INPSRGYTNYNQKVKDRFTISRDNS |
| | | KNTAFLQMDSLRPEDTGVYFCARYY |
| | | DDHYCLDYWGQGTPVTVSS |
| TA3 VH | 27 | EVQLVESGGGLVQPGGSLRLSCAAS |
| | | GYSFTGYTMNWVRQAPGKGLEWVAL |
| | | INPYKGVSTYNQKFKDRFTISVDKS |
| | | KNTAYLQMNSLRAEDTAVYYCARSG |
| | | YYGDSDWYFDVWGQGTLVTVSS |
| TA4 VH | 28 | DIKLQQSGAELARPGASVKMSCKTS |
| | | GYTFTRYTMHWVKQRPGQGLEWIGY |
| | | INPSRGYTNYNQKFKDKATLTTDKS |
| | | SSTAYMQLSSLTSEDSAVYYCARYY |
| | | DDHYCLDYWGQGTTLTVSS |
| TA5 VH | 29 | DVQLVQSGAEVKKPGASVKVSCKAS |
| | | GYTFTRYTMHWVRQAPGQGLEWIGY |
| | | INPSRGYTNYADSVKGRFTITTDKS |
| | | TSTAYMELSSLRSEDTATYYCARYY |
| | | DDHYCLDYWGQGTTVTVSS |
| TA6 VH | 30 | EVQLVQSGAEVKKPGASVKVSCKAS |
| | | GYTFTNYYIHWVRQAPGQGLEWIGW |
| | | IYPGDGNTKYNEKFKGRATLTADTS |
| | | TSTAYLELSSLRSEDTAVYYCARDS |
| | | YSNYYFDYWGQGTLVTVSS |
| TA7 VH | 31 | EVQLVQSGAEVKKPGASVKVSCKAS |
| | | GFTFTSYYIHWVRQAPGQGLEWIGW |
| | | IYPENDNTKYNEKFKDRVTITADTS |
| | | TSTAYLELSSLRSEDTAVYYCARDG |
| | | YSRYYFDYWGQGTLVTVSS |
| CEA (VHH) | 18 | EVQLVESGGGFVQAGESLTLSCTSS |
| | | TLTFTPYRMAWYRQAPGKQRDLVAD |
| | | ISSGDGRTTNYADFAKGRFTISRDN |
| | | IKNTVFLRMTNLKPEDTAVYYCNTF |
| | | VSFVGIARSWGQGTQVTVSS |

CD3-targeting antibody (TA1, TA2, TA3, TA4, TA5, TA6, TA7) light chain sequences are preferably those shown in the table below.

TABLE 3
Antibody light chain sequence list

| Antibody | SEQ ID | |
|---|---|---|
| Light Chain | NO. | Sequence |
| TA1 VL | 32 | QAVVTQESALTTSPGETVTLTCRSS |
| | | TGAVTTSNYANWVQEKPDHLFTGLI |
| | | GGTNKRAPGVPARFSGSLIGDKAAL |
| | | TITGAQTEDEAIYFCALWYSNLWVF |
| | | GGGTKVEIK |
| TA2 VL | 33 | DIQMTQSPSSLSASVGDRVTITCSA |
| | | SSSVSYMNWYQQTPGKAPKRWIYDT |
| | | SKLASGVPSRFSGSGSGTDYTFTIS |
| | | SLQPEDIATYYCQQWSSNPFTFGQG |
| | | TKLQIT |
| TA3 VL | 34 | DIQMTQSPSSLSASVGDRVTITCRA |
| | | SQDIRNYLNWYQQKPGKAPKLLIYY |
| | | TSRLESGVPSRFSGSGSGTDYTLTI |
| | | SSLQPEDFATYYCQQGNTLPWTFGQ |
| | | GTKVEIK |
| TA4 VL | 35 | DIQLTQSPAIMSASPGEKVTMTCRA |
| | | SSSVSYMNWYQQKSGTSPKRWIYDT |
| | | SKVASGVPYRFSGSGSGTSYSLTIS |
| | | SMEAEDAATYYCQQWSSNPLTFGAG |
| | | TKLELK |
| TAS VL | 36 | DIVLTQSPATLSLSPGERATLSCRA |
| | | SQSVSYMNWYQQKPGKAPKRWIYDT |
| | | SKVASGVPARFSGSGSGTDYSLTIN |
| | | SLEAEDAATYYCQQWSSNPLTFGGG |
| | | TKVEIK |
| TA6 VL | 37 | DIVMTQSPDSLAVSLGERATINCKS |
| | | SQSLLNSRTRKNYLAWYQQKPGQPP |
| | | KLLIYWASTRESGVPDRFSGSGSGT |
| | | DFTLTISSLQAEDVAVYYCTQSFIL |
| | | RTFGQGTKVEIK |
| TA7 VL | 38 | DIVMTQSPDSLAVSLGERATINCKS |
| | | SQSLLNSRTRKNYLAWYQQKPGQSP |
| | | KLLIYWTSTRKSGVPDRFSGSGSGT |
| | | DFTLTISSLQAEDVAVYYCKQSFIL |
| | | RTFGQGTKVEIK |

Plasmid construction: All genes involved in this application were synthesized by Nanjing Genscript Biotechnology Co., Ltd. (Genscript), and then were inserted into a pEE12.4 expression vector by restriction enzyme digestion. The plasmid was extracted with OMEGA's plasmid large-scale extraction kit and stored at −80° C.

### Example 2. Preparation of Bispecific Antibodies with Humanized C17 as an Anti-CEA Antibody and Humanized TA1 as an Anti-CD3 Antibody

The variable regions of the heavy and light chain DNA sequences were cloned into a mammalian expression vector, in frame with the pre-inserted constant human IgG4 heavy chain or kappa constant light chain.

The molecule was produced by co-transfecting Expi 293F™ cells (ThermoFisher) with a mammalian expression vector using polyethyleneimine (PEI, POLYETHYLENEIMINE′MAX; polysciences, 24765-2). Cells were transfected with the corresponding expression vector at a ratio of 1:2:1 for “vector heavy chain Fc (hole)”, “vector light chain”, and “vector heavy chain Fc (knob)”.

Expi 293F™ cells were cultured in CD OptiCHO™ medium at 37° C., 5% CO₂, 135 rpm suspension culture. The day before transfection, 293F cells were passaged to 1 L air-permeable conical culture flasks. The cell seeding density was 1.0×10⁶ cells/ml, and the cell volume was 200 mL. The expected cell density on the day of transfection was 1.8-2.0×10⁶ cells/ml. The cell suspension was centrifuged at 1000 rpm at room temperature for 5 min, and the cells were collected. The cells were washed with Expi293 medium once; resuspended with 200 ml Expi293 medium. 400 μg plasmid was diluted with 5 ml Opti-MEM medium, vortexed for 15 seconds. 1.2 mg PEI was diluted with 5 ml Opti-MEM medium and vortex for 15 seconds. The PEI solution was added to the DNA-containing solution, mixed gently, and incubated at room temperature for 15 minutes. The plasmid/PEI mixture was added to the cell suspension, and incubated in a 37° C., 5% CO₂, 85 rpm incubator. 4 hours later, 200 ml EX-CELL™293 medium and 2 mM Glutamine (Gibco) were added, and the incubator was adjust 135 rpm to continue the culture. 24 hours later, 3.8 mM cell proliferation inhibitor VPA was added. 72 hours later, 40 ml medium D was added to continue the culturing. After 7 days of culture, the supernatant was collected by centrifugation at 18000 rpm for 30 min. The solution was aseptically filtered with a 0.22 m filter and sodium azide was added at a final concentration of 0.01% w/v. The solution was stored at 4° C.

Protein A was used to purify the target protein. The cell culture supernatant was loaded onto Mabselect Prism A FF (GE; 17-5498-01) medium equilibrated with 20 ml 25 mM Tris, 150 mM NaCl, pH 7.5. Unbound protein was removed by using at least 10 column volumes of 25 mM Tris, 150 mM NaCl, pH 7.5. The target protein was eluted with 5 column volumes of 20 mM Na-Citrate, pH 3.5. The protein solution was neutralized by adding 1/10 volume of 1 M Tris, pH 9.5.

Zeba™ desalting spin column (ThermoFisher) or ultrafiltration tube (Millipore) was used to place the target protein into the required buffer. The protein concentration and purity were determined by SDS-PAGE electrophoresis and NanoDrop2000, and the protein was aliquoted and stored at −80° C. 2-3 μg samples were used for SDS-PAGE electrophoresis.

The target protein was concentrated and filtered, and then added to a gel filtration column (Gel Filtration, HiLoad Superdex 200, GE). The gel filtration column was equilibrated with a pH 6.0 solution of 20 mM histidine and 140 mM sodium chloride. The target protein was analyzed in PBS buffer (pH 7.2) at room temperature, at a flow rate of 0.5 ml/min to determine characteristics like the molecular weight, purity, aggregation, etc.

In an alternative purification method, the protein was purified from the cell supernatant by protein A affinity chromatography (MabSelect SuRe, GE). The protein eluate was then subjected to cation exchange chromatography (HiTrap SP HP, GE) and then passed through a gel filtration layer column (SEC) for fractionation and analysis. The protein obtained by this purification method has a purity of >90% of the target antibody content.

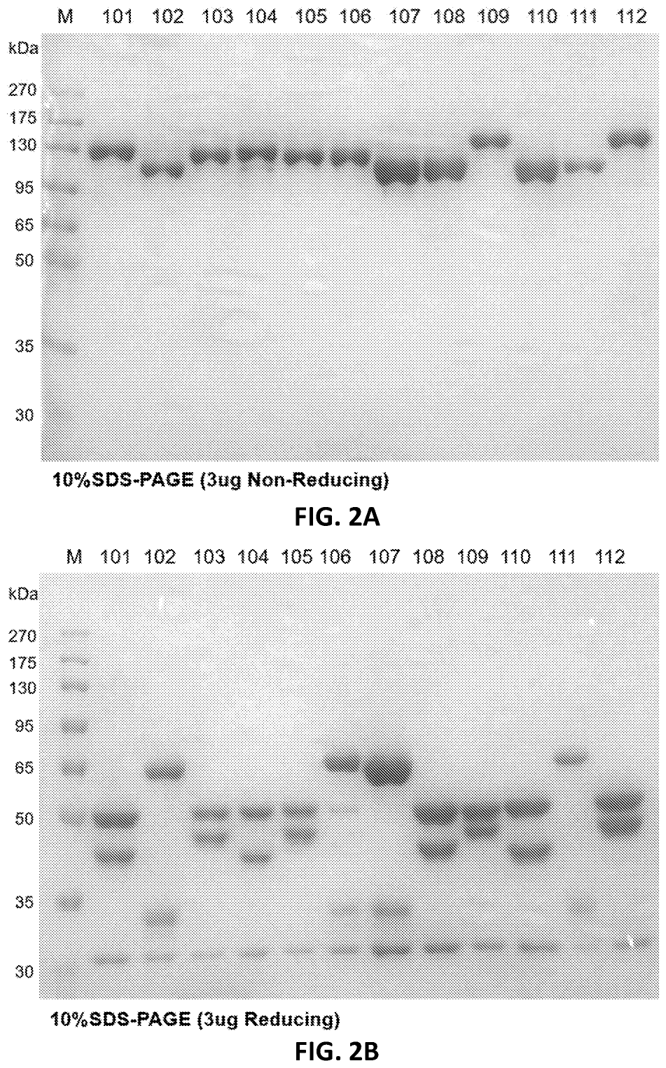

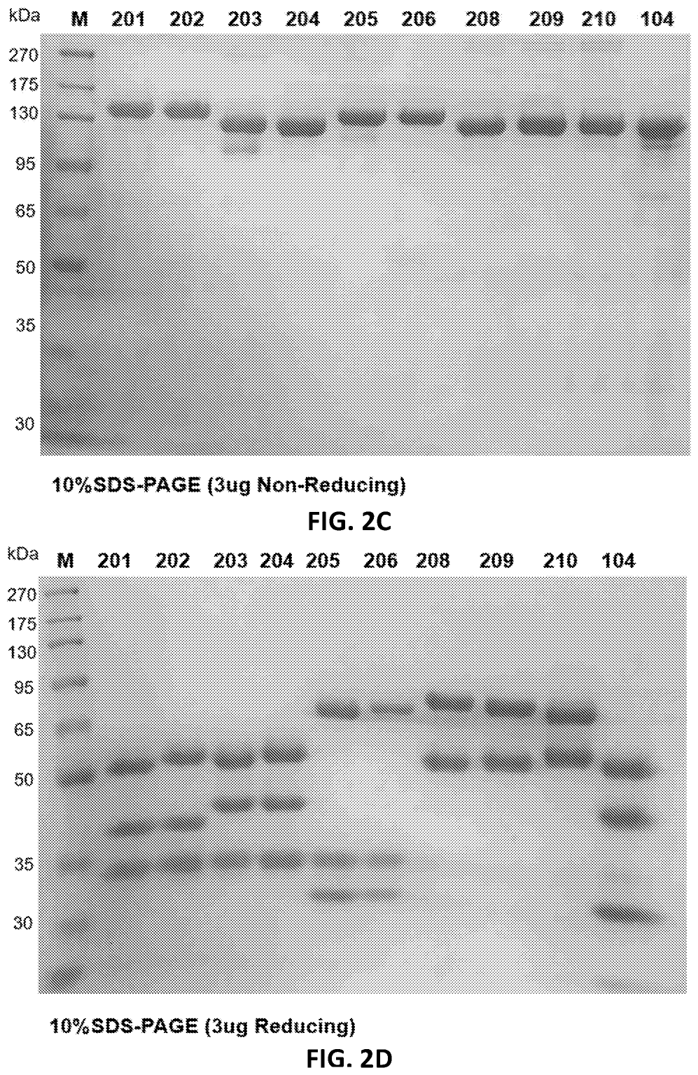

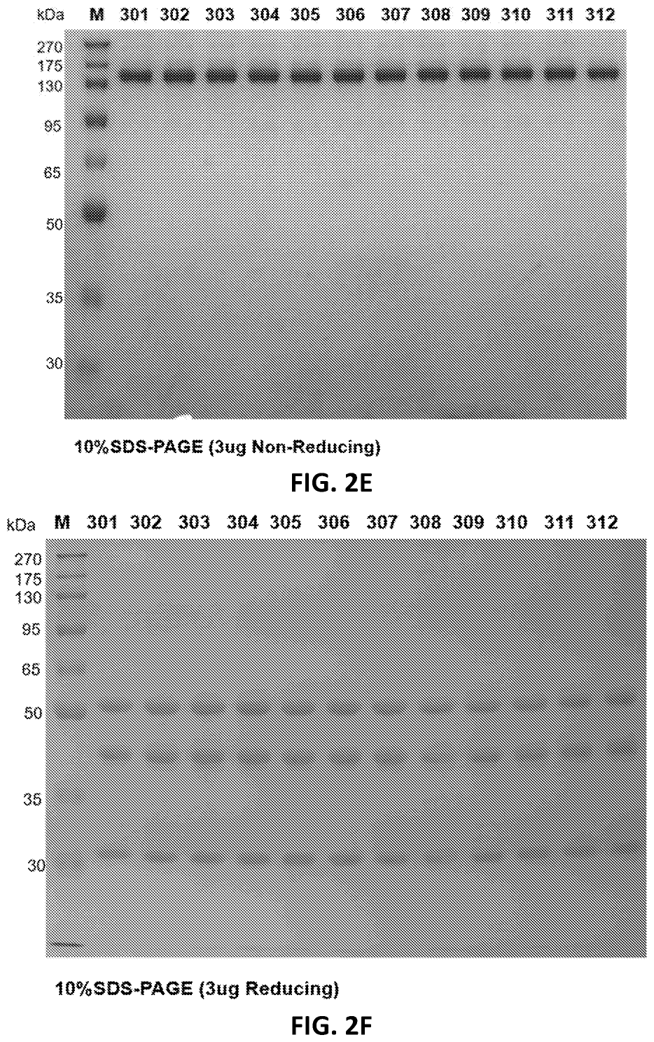

The protein structure design is shown in FIGS. 1A-1X, and the SDS-PAGE result is shown in FIGS. 2A-2F; the SEC analysis result of protein 101-112 is shown in the table below.

The protein yield was about 20 mg/L, and the yield of protein 207, 208, 209 and 210 was less than 10 mg/L.

TABLE 4
Yield and purity of bispecific antibodies

| | | Pu- | | | Pu- | | | Pu- |
|---|---|---|---|---|---|---|---|---|
| Sam- | Yield | rity | Sam- | Yield | rity | Sam- | Yield | rity |
| ple | (mg/L) | % | ple | (mg/L) | % | ple | (mg/L) | % |
| 101 | 22.19 | 99.97 | 201 | 12.62 | 96.35 | 301 | 34.58 | 96.32 |
| 102 | 21.34 | 93.51 | 202 | 15.8 | 97.11 | 302 | 26.08 | 95.62 |
| 103 | 18.8 | 94.25 | 203 | 6.55 | 90.02 | 303 | 18.64 | 96.11 |
| 104 | 21.8 | 94.39 | 204 | 13.43 | 94.32 | 304 | 45.5 | 96.82 |
| 105 | 20.0 | 93.51 | 205 | 14.12 | 92.34 | 305 | 22.8 | 96.46 |
| 106 | 30.4 | 95.45 | 206 | 21.97 | 95.20 | 306 | 23.9 | 93.94 |
| 107 | 15.2 | 94.45 | 207 | 5.53 | 95.00 | 307 | 33.9 | 97.23 |
| 108 | 17.4 | 94.34 | 208 | 2.22 | 94.98 | 308 | 21.26 | 96.36 |
| 109 | 19.6 | 92.28 | 209 | 5.9 | 95.42 | 309 | 22.56 | 97.46 |
| 110 | 14.5 | 92.04 | 210 | 5.43 | 94.72 | 310 | 24.86 | 96.95 |
| 111 | 16.4 | 92.16 | | | | 311 | 23.46 | 96.82 |
| 112 | 19.6 | 93.23 | | | | 312 | 12.6 | 97.89 |

### Example 3. Binding of Bispecific Antibodies to CEA and CD3 Expressing Cells

The binding of the bispecific antibodies was tested on the CA expressing human lung adenocarcinoma cell line (H1573) and the CD3 expressing immortalized T lymphocyte line (Jurkat). The steps are as follows: the cells were harvested, counted, and resuspended in FACS buffer (PBS containing 0.1% BSA) at 2×10⁶ cells/ml. 100 μl of cell suspension was incubated in a V-bottom 96-well plate at 4° C. with 3-fold serial dilutions of bispecific antibodies (20 μg/ml-0.009 μg/ml) for 30 minutes; washed twice with pre-cooled FACS buffer. The goat anti-human IgG Fcγ fragment-specific secondary antibody (eBioscience, 12-4998-82) conjugated to PE was incubated at 4° C. for 30 minutes, washed 3 times with pre-cooled FACS buffer, followed by FACS analysis using a flow cytometry cell analyzer (Beckman, Cytoflex). GraphPadPrism was used to obtain the binding curve.

For the convenience of statistical analysis, the average cell fluorescence intensity (MFI) of the bispecific antibodies at 20 μg/ml was set as 100%, and the EC50 of the bispecific antibodies and the cell binding was obtained by fitting the percentage of the cell fluorescence value at different concentrations.

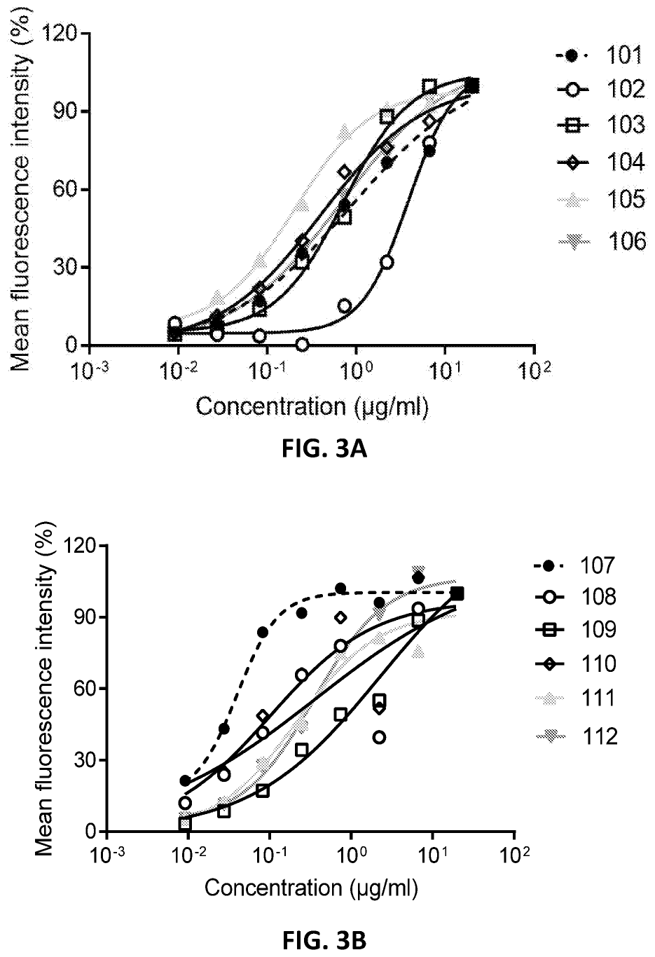

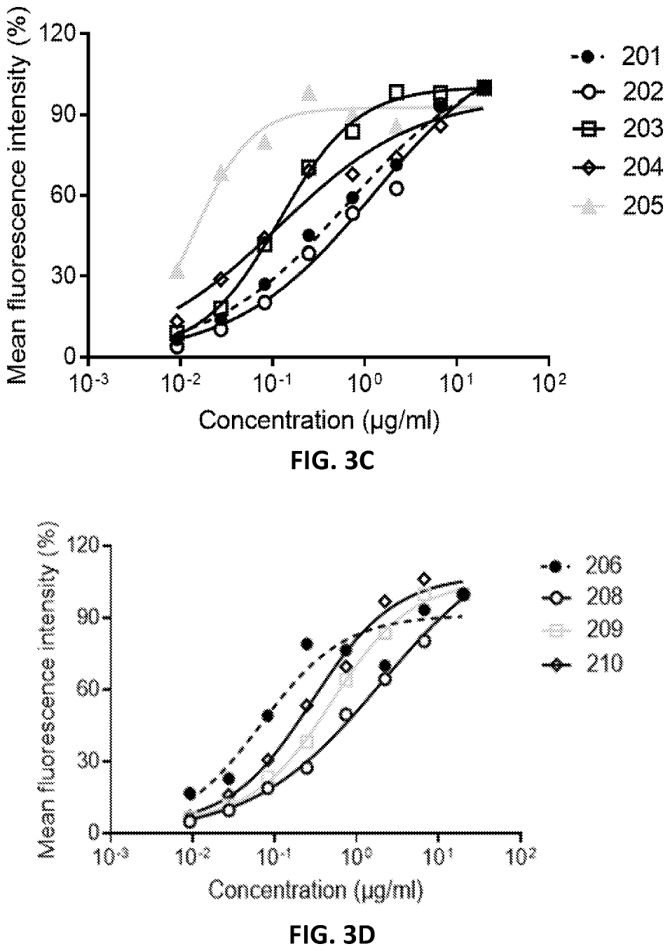

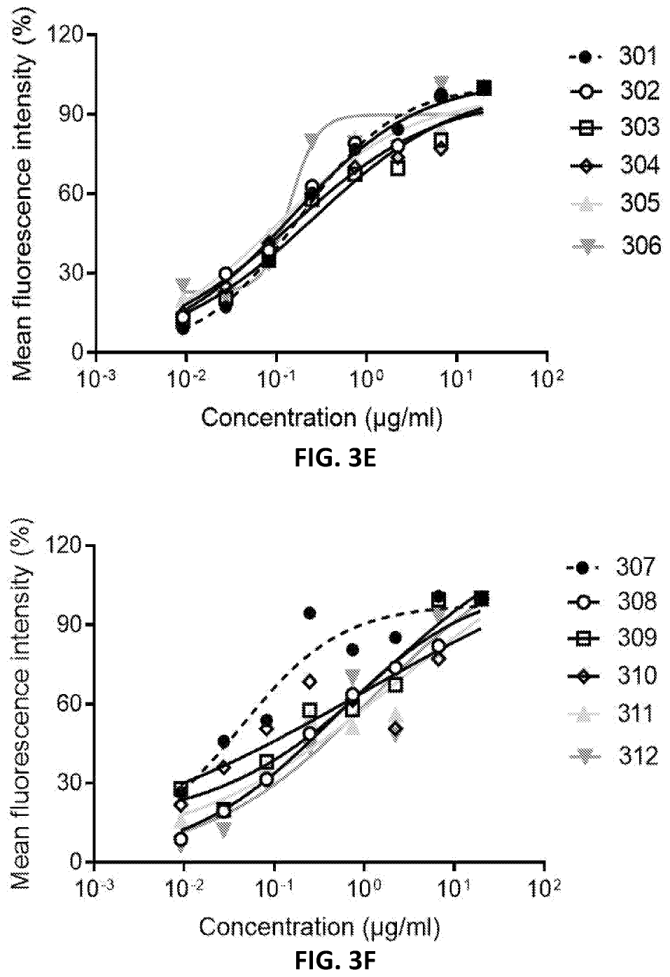

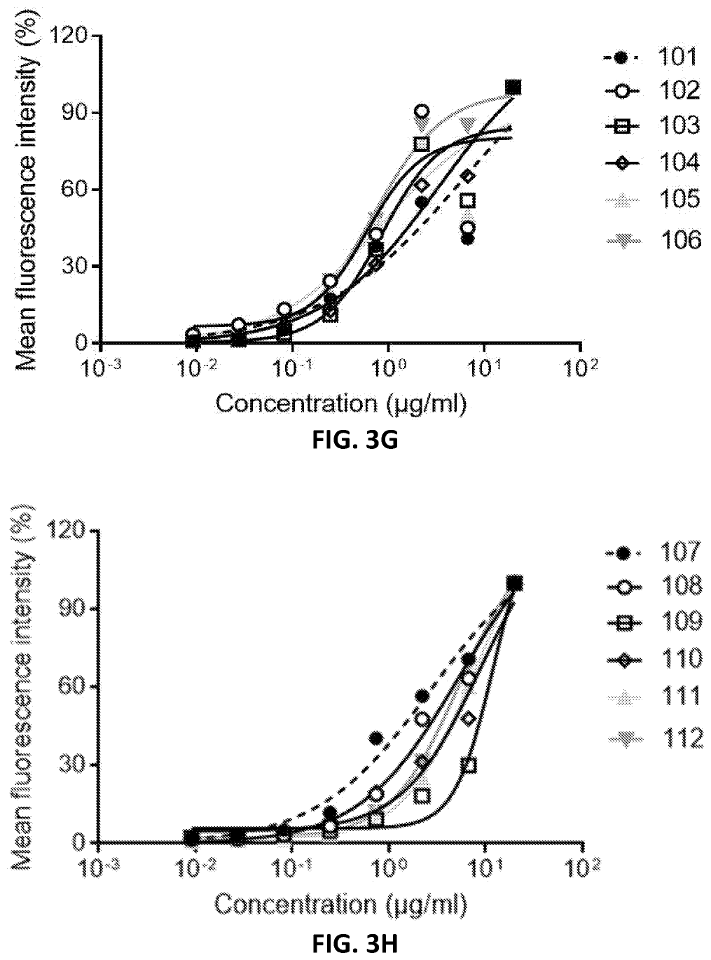

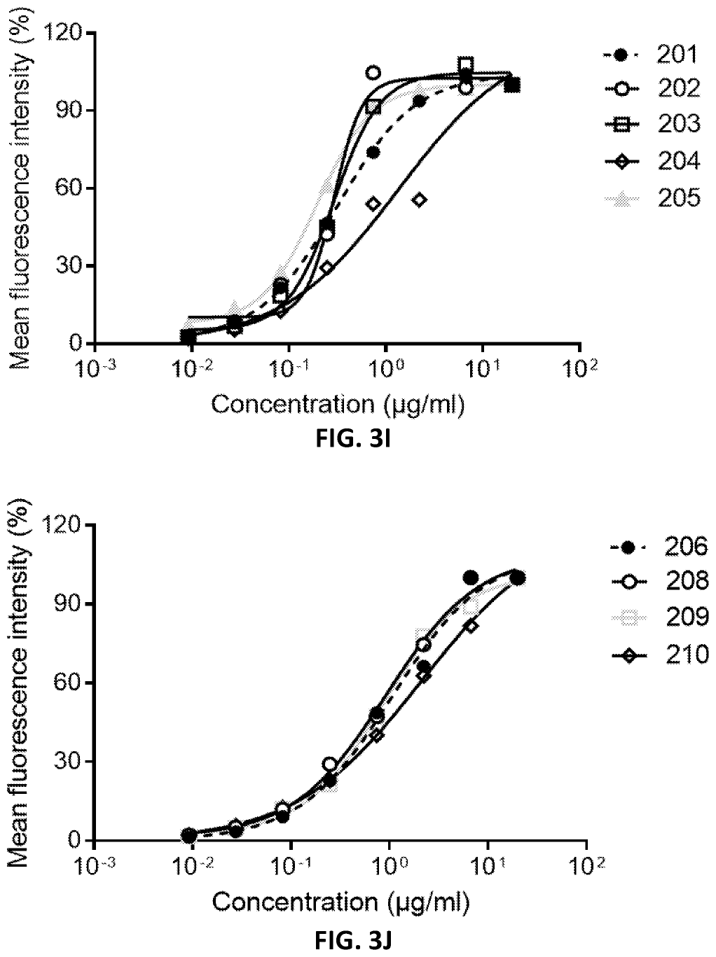

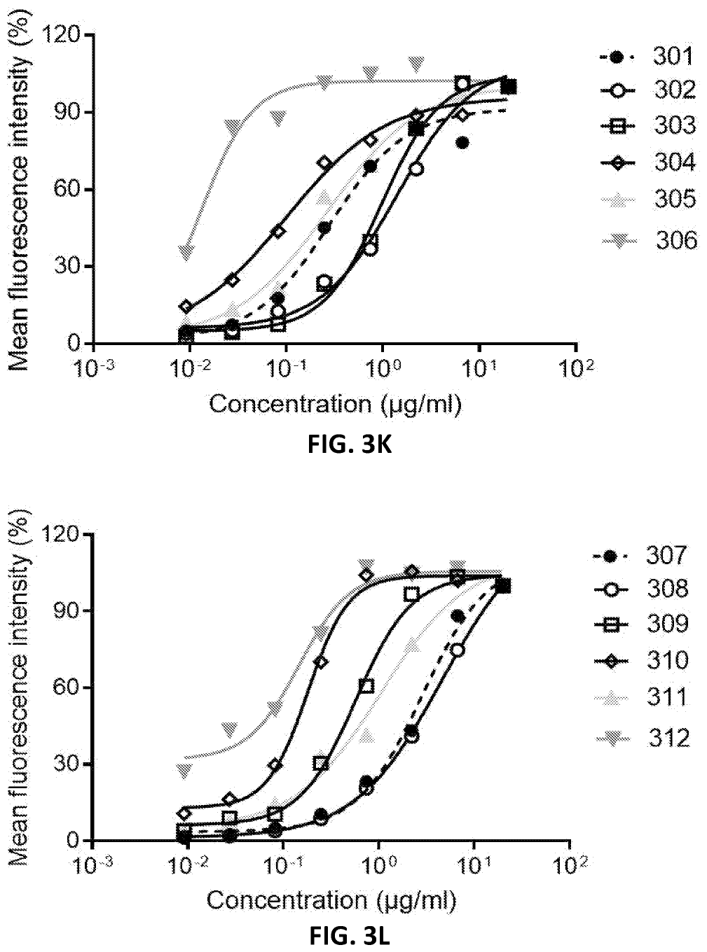

It can be seen from FIGS. 3A-3L that the bispecific antibodies bind well to the CEA expressing cells and CD3 expressing cells, and the cell binding EC50 is shown in the tables below.

TABLE 5
EC50 of bispecific antibody binding to H1573 cells

| | H1573 Cell Binding | | | | | | | |
|---|---|---|---|---|---|---|---|---|
| Sam- | EC50 | EC50 | Sam- | EC50 | EC50 | Sam- | EC50 | EC50 |
| ple | (μg/ml) | (nM) | ple | (μg/ml) | (nM) | ple | (μg/ml) | (nM) |
| 101 | 0.8855 | 6.81 | 201 | 0.6478 | 4.98 | 301 | 0.1774 | 1.36 |
| 102 | 3.875 | 29.81 | 202 | 1.27 | 9.77 | 302 | 0.1496 | 1.15 |
| 103 | 0.7199 | 5.54 | 203 | 0.1198 | 0.92 | 303 | 0.2555 | 1.97 |
| 104 | 0.4084 | 3.14 | 204 | 0.1173 | 0.90 | 304 | 0.157 | 1.21 |
| 105 | 0.2025 | 1.56 | 205 | 0.01412 | 0.11 | 305 | 0.1047 | 0.81 |
| 106 | 0.6914 | 5.32 | 206 | 0.06778 | 0.52 | 306 | 0.1418 | 1.09 |
| 107 | 0.03982 | 0.31 | 208 | 2.005 | 15.42 | 307 | 0.04341 | 0.33 |
| 108 | 0.3676 | 2.83 | 209 | 0.5033 | 3.87 | 308 | 0.4287 | 3.30 |
| 109 | 2.259 | 17.38 | 210 | 0.2899 | 2.23 | 309 | 1.636 | 12.58 |
| 110 | 0.1016 | 0.78 | | | | 310 | 1.026 | 7.89 |
| 111 | 0.2161 | 1.66 | | | | 311 | 2.002 | 15.40 |
| 112 | 0.3497 | 2.69 | | | | 312 | 1.236 | 9.51 |

TABLE 6
EC50 of bispecific antibody binding to Jurkat cells

| | Jurkat Cell Binding | | | | | | | |
|---|---|---|---|---|---|---|---|---|
| Sam- | EC50 | EC50 | Sam- | EC50 | EC50 | Sam- | EC50 | EC50 |
| ple | (μg/ml) | (nM) | ple | (μg/ml) | (nM) | ple | (μg/ml) | (nM) |
| 101 | 6.277 | 48.28 | 201 | 0.3124 | 2.40 | 301 | 0.2817 | 2.17 |
| 102 | 0.5921 | 4.55 | 202 | 0.2922 | 2.25 | 302 | 1.399 | 10.76 |
| 103 | 0.8481 | 6.52 | 203 | 0.2898 | 2.23 | 303 | 0.9779 | 7.52 |
| 104 | 3.353 | 25.79 | 204 | 1.358 | 10.45 | 304 | 0.1006 | 0.77 |
| 105 | 0.8497 | 6.54 | 205 | 0.1946 | 1.50 | 305 | 0.2668 | 2.05 |
| 106 | 0.6903 | 5.31 | 206 | 1.078 | 8.29 | 306 | 0.01289 | 0.10 |
| 107 | 2.801 | 21.55 | 208 | 0.9175 | 7.06 | 307 | 2.86 | 22.00 |
| 108 | 5.531 | 42.55 | 209 | 0.8663 | 6.66 | 308 | 4.593 | 35.33 |
| 109 | 12.09 | 93.00 | 210 | 2.076 | 15.97 | 309 | 0.5753 | 4.43 |
| 110 | 8.876 | 68.28 | | | | 310 | 0.1809 | 1.39 |
| 111 | 7.599 | 58.45 | | | | 311 | 1.042 | 8.02 |
| 112 | 5.32 | 40.92 | | | | 312 | 0.1462 | 1.12 |

### Example 4. Determining the Affinity of Bispecific Antibodies to Human CEA and Human CD3 ε/δ Through Bio-Layer Interferometry (BLI)

Biofilm interference (BLI) experiments were conducted using OCTECT RED96e (ForteBijo) at 30° C. with 0.02% PBST solution as the running buffer (10 mmol/L Na₂HPO₄; 1.75 mmol/L KH₂PO₄; 137 mmol/L NaCl; 2.65 mmol/L KCl; pH 7.2-7.4, 0.02% Surfactant Tween 20).

First, the bispecific antibodies were attached onto the surface of the AHIC sensor (Anti-hIgG Fc, sartorius, 18-5060) with immobilized anti-human Fc. The bispecific antibodies were diluted to 5 ag/ml, and 1.5 nm protein was coupled to the surface of the AHIC sensor chip through 0.02% PBST.

Analytes human CD3ε/δ (ACRO systems, CDD-H52W1) and recombinant tumor antigen human CEA (CEACAM-5/CD66 e, ACRO systems, CE5-H5226) were diluted in 0.02% PBST solution to the below concentrations: 200 nM, 100 nM, 50 nM, 25 nM, 12.5 nM, 6.25 nM, 3.125 nM.

After the chip captures the bispecific antibodies, the chip binds and dissociates with different concentrations of analyte to obtain the dissociation constant (KD) value of the interaction. The experimental parameters are as follows: Baseline1: 60s, Loading: 240s, Baseline2: 180s, Association: 240s, Dissociation: 600s, High Sensitivity kinetics: 2 Hz. The kinetic constants were derived by fitting the rate equation of 1:1 Langmuir binding by numerical integration, and the equilibrium dissociation constant (KD) was obtained. Data Analysis HT 12 software (sartorius, 50-5029) simultaneously fits the binding and dissociation curve to calculate Kₒₙ and K_{off}, K_{D}=K_{off}/Kₒₙ.

The experimental results showed that the affinity of the bispecific antibodies to CEA and CD3 was between 0.13 nM-3.6 nM, and the affinity was relatively high. The results of the binding of the bispecific antibodies with different concentrations of antigen are shown in the tables below.

TABLE 7
Affinity of bispecific antibodies to human CEA (T = 30° C.)

| | kₐ | k_{dis} | K_{D} | | kₐ | k_{dis} | K_{D} | | kₐ | k_{dis} | K_{D} |
|---|---|---|---|---|---|---|---|---|---|---|---|
| Sample | (1/Ms) | (1/s) | (M) | Sample | (1/Ms) | (1/s) | (M) | Sample | (1/Ms) | (1/s) | (M) |
| 101 | 2.68E+05 | 1.02E−04 | 3.80E−10 | 201 | 1.47E+05 | 8.29E−05 | 5.65E−10 | 301 | 4.91E+05 | 6.61E−05 | 1.34E−10 |
| 102 | 4.77E+05 | 6.49E−05 | 1.36E−10 | 202 | 1.11E+05 | 1.34E−04 | 1.21E−09 | 302 | 5.28E+05 | 9.03E−05 | 1.71E−10 |
| 103 | 1.47E+05 | 1.67E−04 | 1.13E−09 | 203 | 3.50E+05 | 1.10E−04 | 3.15E−10 | 303 | 5.52E+05 | 9.50E−05 | 1.72E−10 |
| 104 | 2.74E+05 | 1.35E−04 | 4.93E−10 | 204 | 3.49E+05 | 1.08E−04 | 3.09E−10 | 304 | 5.14E+05 | 8.69E−05 | 1.69E−10 |
| 105 | 1.54E+05 | 1.90E−04 | 1.24E−09 | 205 | 3.14E+05 | 1.00E−04 | 3.20E−10 | 305 | 5.36E+05 | 9.27E−05 | 1.73E−10 |
| 106 | 1.86E+05 | 1.49E−04 | 8.03E−10 | 206 | 1.95E+05 | 8.88E−05 | 4.56E−10 | 306 | 5.52E+05 | 1.12E−04 | 2.04E−10 |
| 107 | 6.29E+04 | 3.65E−04 | 5.80E−09 | 208 | 1.92E+05 | 8.24E−05 | 4.29E−10 | 307 | 5.99E+05 | 1.06E−04 | 1.77E−10 |
| 108 | 3.75E+05 | 4.68E−05 | 1.25E−10 | 209 | 2.01E+05 | 1.09E−04 | 5.40E−10 | 308 | 5.76E+05 | 1.17E−04 | 2.02E−10 |
| 109 | 1.27E+05 | 1.61E−04 | 1.27E−09 | 210 | 3.32E+05 | 9.09E−05 | 2.74E−10 | 309 | 5.89E+05 | 1.23E−04 | 2.09E−10 |
| 110 | 3.60E+05 | 6.64E−05 | 1.84E−10 | | | | | 310 | 6.03E+05 | 1.40E−04 | 2.33E−10 |
| 111 | 4.01E+05 | 8.41E−05 | 2.10E−10 | | | | | 311 | 6.00E+05 | 1.25E−04 | 2.09E−10 |
| 112 | 1.69E+05 | 7.32E−05 | 4.32E−10 | | | | | 312 | 6.37E+05 | 1.28E−04 | 2.01E−10 |

TABLE 8
Affinity of bispecific antibodies to human CD3ϵ/CD3δ (T=30° C.)

| | kₐ | k_{dis} | K_{D} | | kₐ | k_{dis} | K_{D} | | kₐ | k_{dis} | K_{D} |
|---|---|---|---|---|---|---|---|---|---|---|---|
| Sample | (1/Ms) | (1/s) | (M) | Sample | (1/Ms) | (1/s) | (M) | Sample | (1/Ms) | (1/s) | (M) |
| 101 | 4.09E+05 | 4.00E−04 | 9.79E−10 | 201 | 1.78E+05 | 4.60E−04 | 2.59E−09 | 301 | 5.36E+04 | 1.32E−03 | 1.02E−09 |
| 102 | 3.33E+05 | 3.27E−04 | 9.84E−10 | 202 | 2.22E+05 | 8.66E−04 | 3.90E−09 | 302 | 2.46E+03 | 8.79E−04 | 3.63E−09 |
| 103 | 3.52E+05 | 2.33E−04 | 6.62E−10 | 203 | 2.48E+05 | 6.21E−04 | 2.51E−09 | 303 | 2.35E+05 | 2.41E−03 | 6.45E−10 |
| 104 | 3.57E+05 | 3.00E−04 | 8.39E−10 | 204 | 1.97E+05 | 5.71E−04 | 2.91E−09 | 304 | 2.81E+05 | 2.37E−03 | 6.76E−10 |
| 105 | 3.67E+05 | 2.56E−04 | 6.96E−10 | 205 | 2.79E+05 | 6.66E−04 | 2.38E−09 | 305 | 3.24E+03 | 8.70E−04 | 2.85E−09 |
| 106 | 3.66E+05 | 2.15E−04 | 5.89E−10 | 206 | 1.80E+05 | 5.52E−04 | 3.08E−09 | 306 | 2.78E+03 | 4.19E−04 | 1.40E−09 |
| 107 | 3.60E+05 | 8.03E−04 | 2.23E−09 | 208 | 2.28E+05 | 6.42E−04 | 2.82E−09 | 307 | 1.10E+05 | 2.60E−03 | 1.39E−09 |
| 108 | 2.62E+05 | 4.70E−04 | 1.79E−09 | 209 | 1.68E+05 | 3.38E−04 | 2.01E−09 | 308 | 4.17E+03 | 9.97E−04 | 3.01E−09 |
| 109 | 2.91E+05 | 4.85E−04 | 1.67E−09 | 210 | 2.43E+05 | 6.89E−04 | 2.83E−09 | 309 | 1.09E+05 | 3.27E−03 | 1.71E−09 |
| 110 | 2.85E+05 | 5.65E−04 | 1.98E−09 | | | | | 310 | 1.04E+05 | 2.67E−03 | 1.60E−09 |
| 111 | 3.33E+05 | 6.94E−04 | 2.08E−09 | | | | | 311 | 3.03E+03 | 1.01E−03 | 3.66E−09 |
| 112 | 2.52E+05 | 6.14E−04 | 2.44E−09 | | | | | 312 | 1.57E+03 | 5.65E−04 | 3.24E−09 |

### Example 5. T Cell-Mediated Killing of CEA-Expressing Tumor Target Cells

H1573 (high CEA), BxPC-3 (medium CEA), H1395 (medium CEA), HT29 (medium CEA), and H1650 (low CEA) human tumor cells were used to evaluate T cell mediated cell killing induced by bispecific antibodies. H157 (CEA negative tumor cell line) was used as a negative control. Human PBMC was used as the effector cell and the killing was detected 72 hours after incubation with the bispecific antibodies.

In short, the tumor cells were digested with trypsin/EDTA, washed once with pre-chilled PBS, resuspended in 10% FBS RPMI1640 medium, and spread on a flat-bottom 96-well plate (Corning 3599) at a density of 5,000 cells/well. After incubating for 4 hours, 50 μL of serially diluted bispecific antibody solution was added (3 replicate wells for each concentration). The cells were incubated for 30 minutes to allow the protein to fully bind to the cells. PBMC cells (Leide Bio, 1521) were resuscitated. After thawing quickly, the PBMC cells were added to 10 mL of RPMI 1640 medium containing 10% FBS. After centrifugation at 1000 rpm for 5 minutes, the supernatant was discarded and the cells were resuspended in complete medium. The cell density was adjusted based on need. The PBMC cells were added to the target cells at a final E:T ratio of 10:1 (50 μL of PBMC per well). The 96-well plate was incubated in a 37° C., 5% CO₂ incubator for 3 days.

After the incubation, the cell culture supernatant was carefully aspirated, and the 96-well plate was pat dried on a paper towel. Serum-Free DMEM containing 10% CCK8 detection solution (Dojindo, CK04-20) was added to the 96-well plate, and incubated at 37° C. for 2-4 h. Afterwards, the absorbance at 450 nm was detected in a microplate reader (BioTech, Synergy LX). The cell survival rate was calculated according to the below formula: cell survival rate=[(As−Ab)/(Ac−Ab)]×100%.
As: Experimental well (medium containing cells, CCK-8, test substance)
Ac: control well (medium containing cells, CCK-8, no test substance)
Ab: Blank well (medium without cells and test substance, CCK-8)
At the same time, GraphPad Prism software was used to fit the cell survival rate with four parameters based on the below formula: Y=(A−D)/[1+(X/C)B]+D

A: Asymptotic estimation above the curve; D: Asymptotic estimation under the curve; B: Slope of the curve; C: The corresponding dose when the binding is half of the maximum; where Y is the detected cell survival rate, X is the drug concentration, and C is the half effective concentration (EC50).

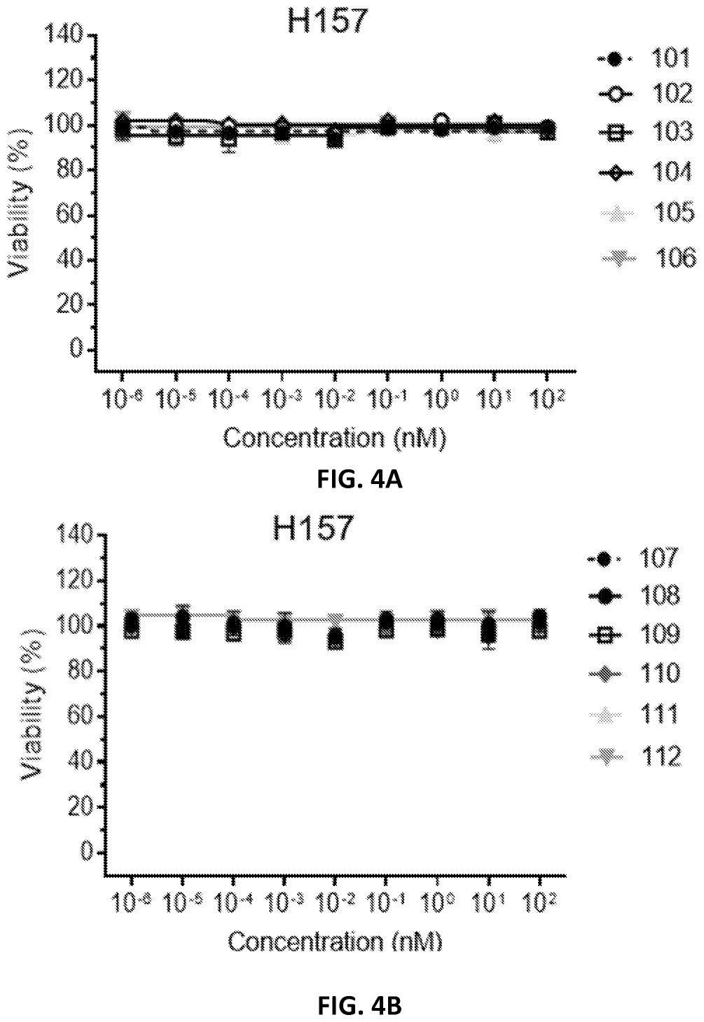

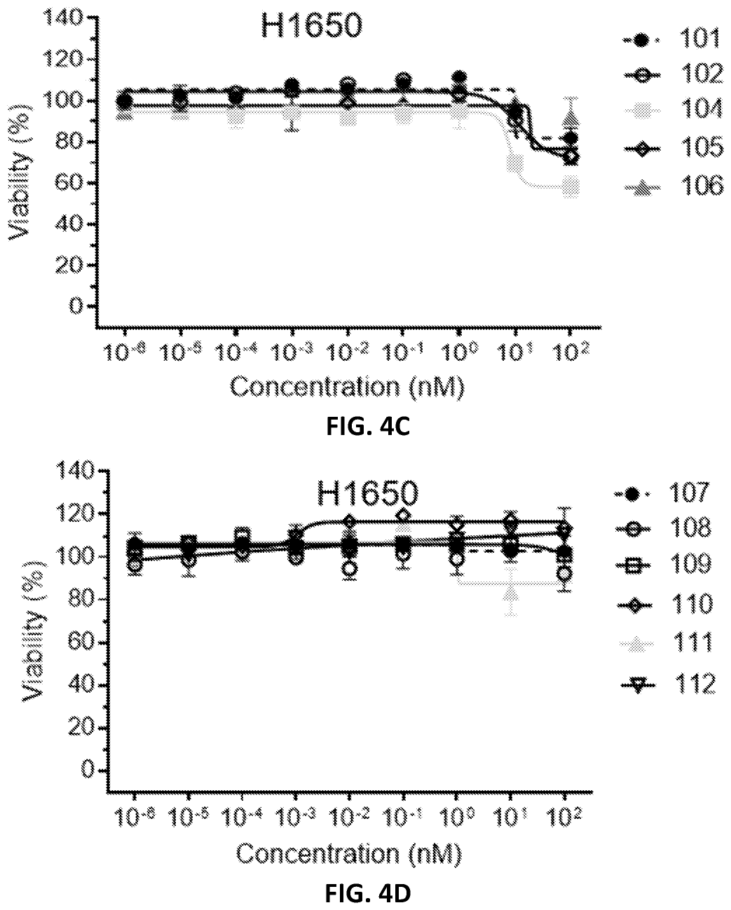

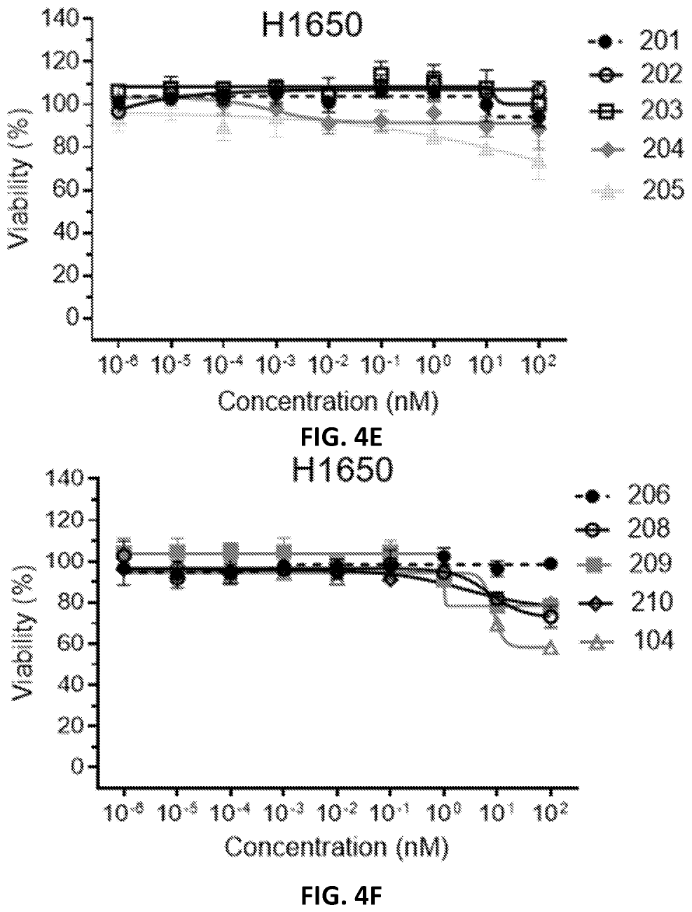

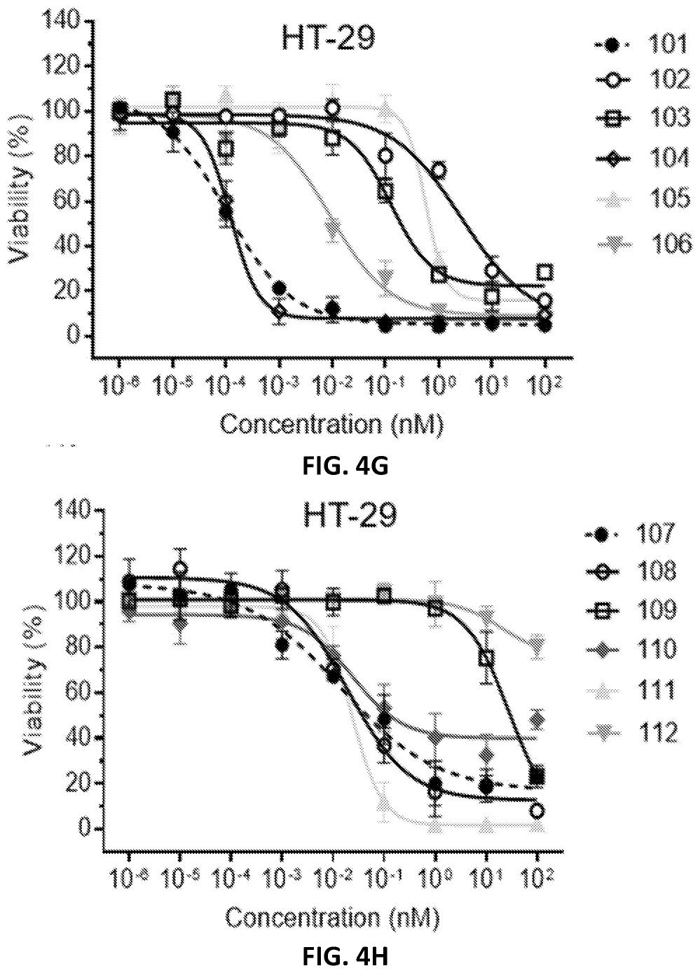

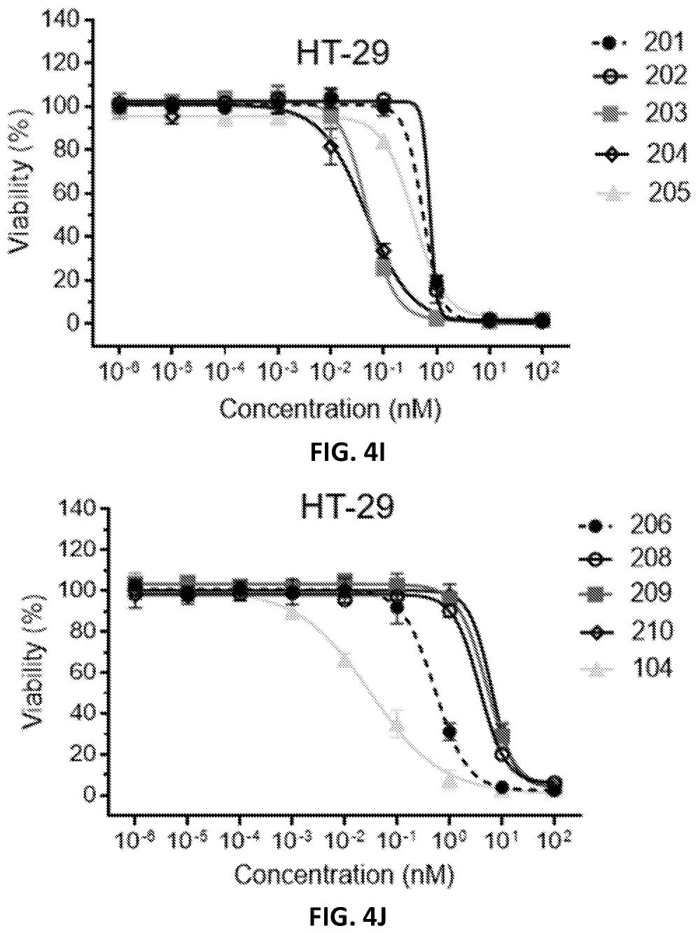

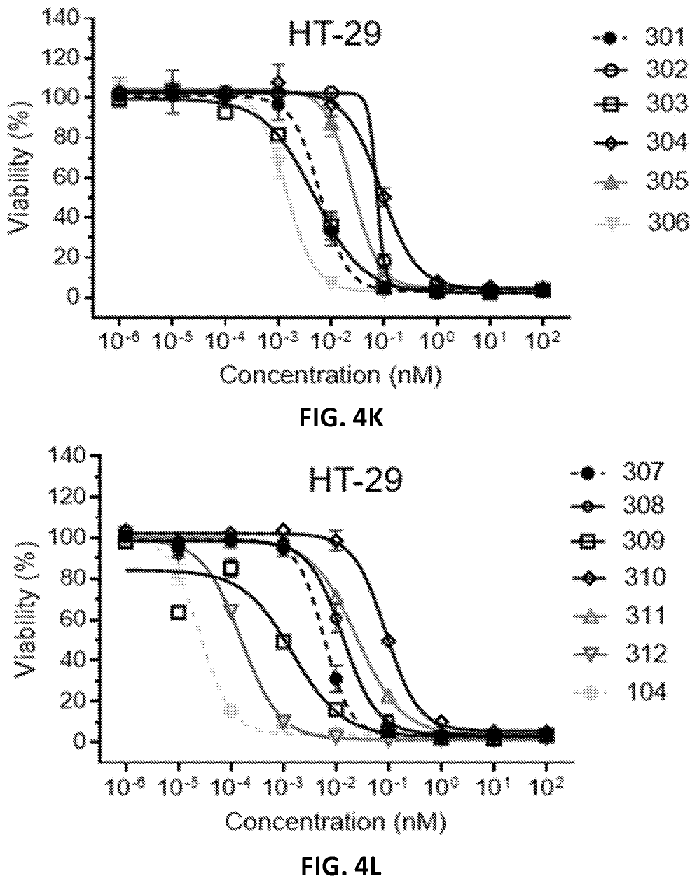

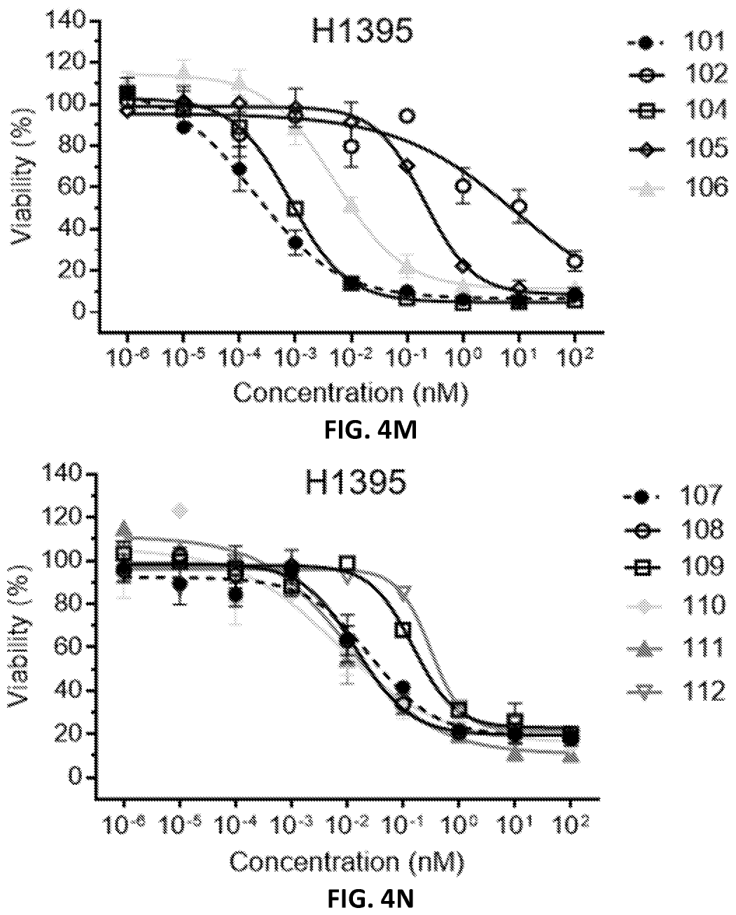

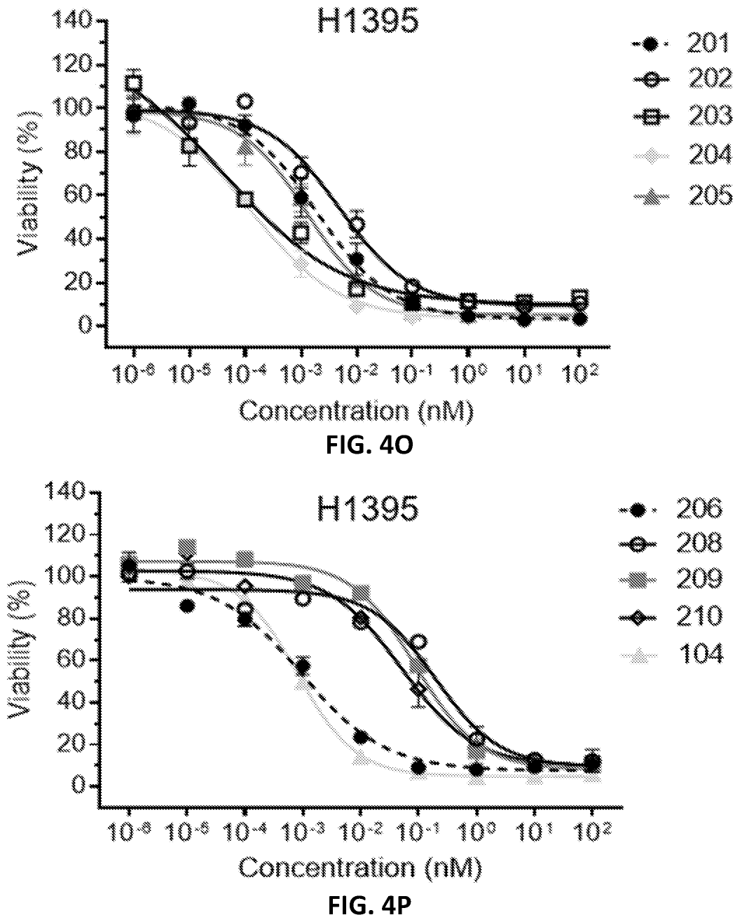

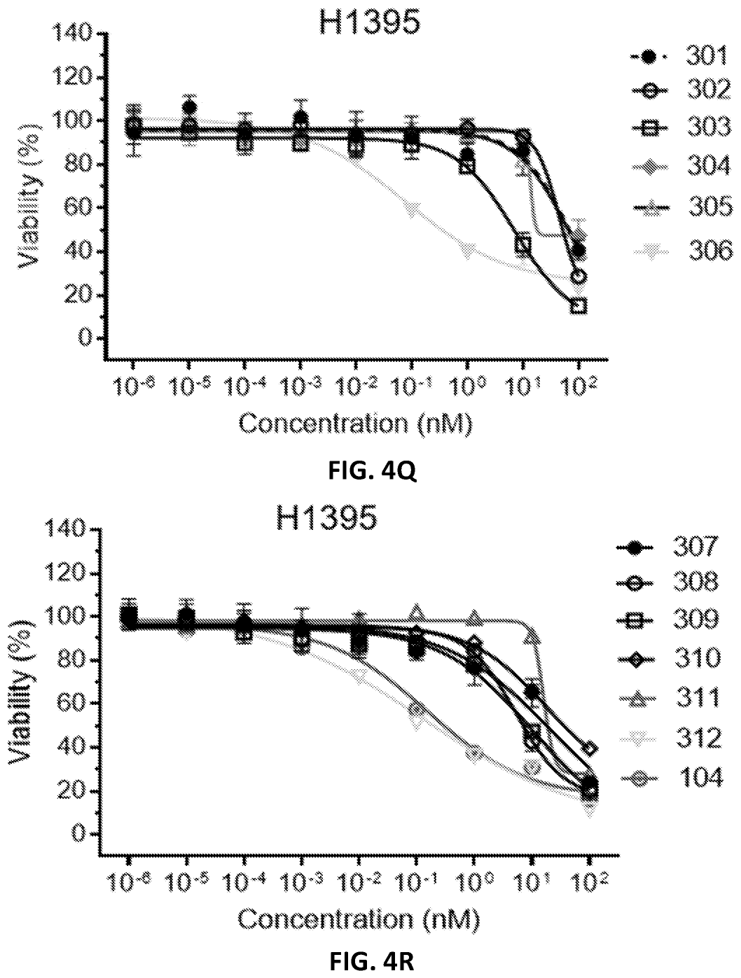

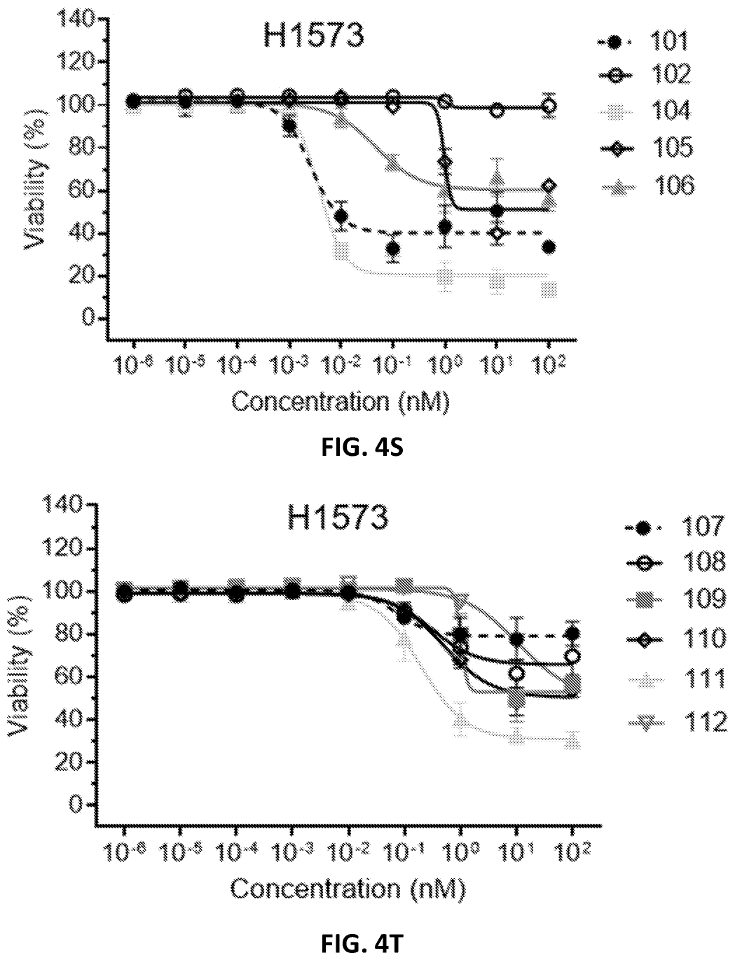

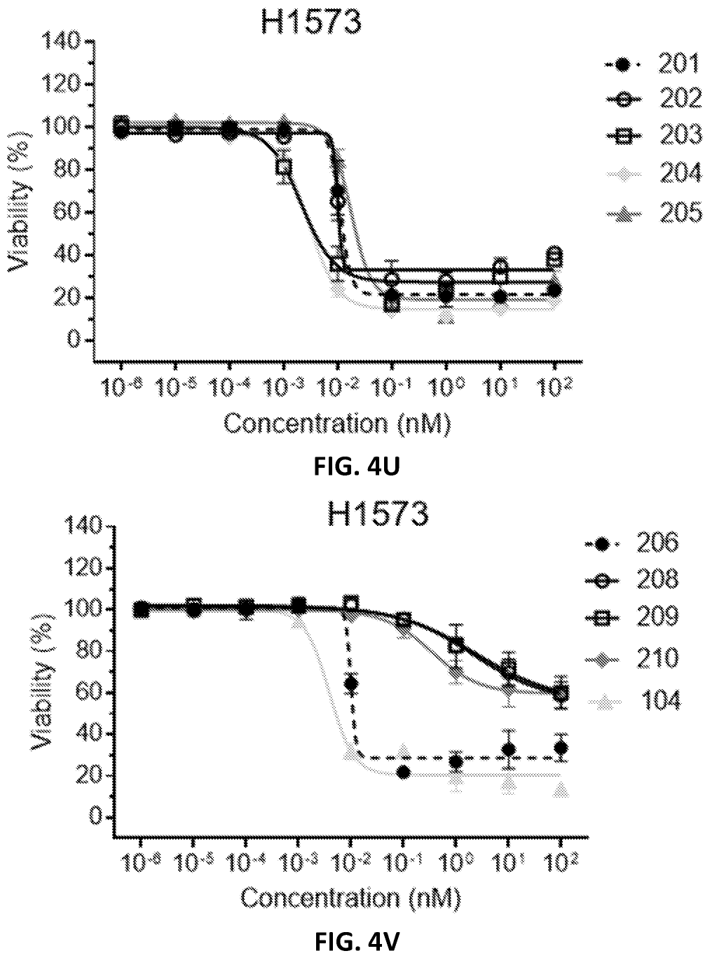

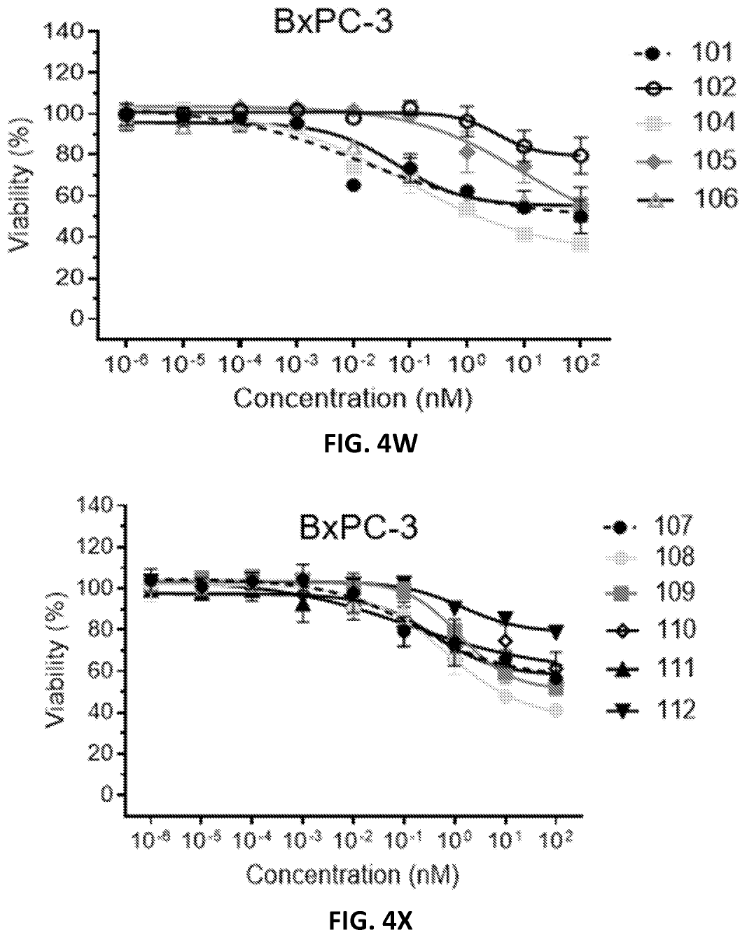

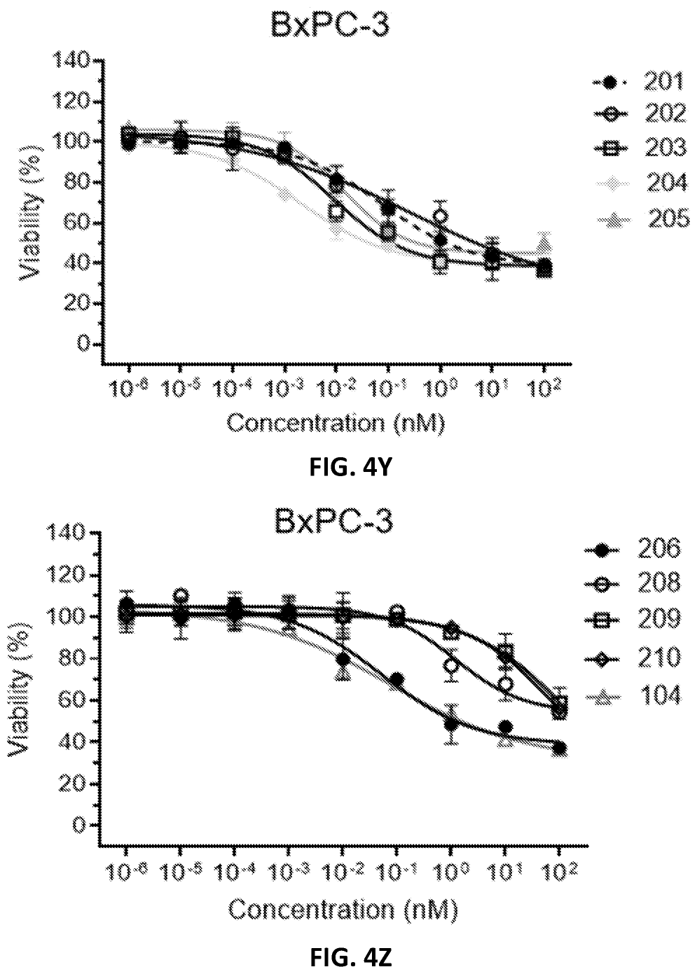

The results showed that the bispecific antibodies induced target-specific killing of CEA-positive tumor cells (FIGS. 4A-4Z). The cell killing ability related EC50 values calculated using GraphPadPrism are shown in the tables below.

TABLE 9
The killing effect of bispecific antibodies
on tumor cells as measured by EC50 (101-112)

| | H157 | H1650 | HT29 | H1395 | H1573 | BXPC3 |
|---|---|---|---|---|---|---|
| Sample | EC50 (nM) | | | | | |
| 101 | >100 | 10.02 | 0.0001023 | 0.0001849 | 0.002669 | 0.01504 |
| 102 | >100 | 11.94 | 2.634 | 9.29 | >100 | 3.459 |
| 103 | >100 | ND | 0.1264 | ND | ND | ND |
| 104 | >100 | 8.353 | 0.0001216 | 0.0008057 | 0.003871 | 0.077249 |
| 105 | >100 | 18.7 | 0.5699 | 0.20 | 0.97 | 9.35 |
| 106 | >100 | >100 | 0.008934 | 0.005164 | 0.04593 | 0.05684 |
| 107 | >100 | >100 | 0.01396 | 0.02633 | 0.07426 | 0.1927 |
| 108 | >100 | >100 | 0.01891 | 0.01461 | 0.3104 | 0.6547 |
| 109 | >100 | >100 | 29.26 | 0.1505 | 1.023 | 1.247 |
| 110 | >100 | >100 | 0.02415 | 0.006998 | 0.4981 | 0.1301 |
| 111 | >100 | >100 | 0.02346 | 0.01026 | 0.2002 | 0.377 |
| 112 | >100 | >100 | 20.76 | 0.3143 | 10.75 | 1.417 |

TABLE 10
The killing effect of bispecific antibodies
on tumor cells as measured by EC50 (201-210)

| | H1650 | HT29 | H1395 | H1573 | BXPC3 |
|---|---|---|---|---|---|
| Sample | EC50 (nM) | | | | |
| 201 | >100 | 0.05499 | 0.001906 | 0.01126 | 0.7676 |
| 202 | >100 | 0.07474 | 0.004899 | 0.01001 | 0.2603 |
| 203 | >100 | 0.004966 | 0.00004188 | 0.002163 | 0.009395 |
| 204 | >100 | 0.004723 | 0.0001215 | 0.002874 | 0.002143 |
| 205 | >100 | 0.03671 | 0.001288 | 0.01757 | 0.01438 |
| 206 | >100 | 0.05285 | 0.0009631 | 0.009983 | 0.05293 |
| 208 | >100 | 0.3765 | 0.1933 | 2.074 | 1.213 |
| 209 | 2.571 | 0.5101 | 0.08807 | 2.3998 | 200.9 |
| 210 | >100 | 0.6334 | 0.0511 | 0.3446 | 60.01 |

TABLE 11
The killing effect of bispecific antibodies
on tumor cells as measured by EC50 (301-312)

| | |
|---|---|
| HT29 | H1395 |

| | |
|---|---|
| Sample | EC50 (nM) |

| | | |
|---|---|---|
| 301 | 0.006128 | 75.1 |
| 302 | 0.07269 | 44.97 |
| 303 | 0.00482 | 7.203 |
| 304 | 0.08852 | 13.64 |
| 305 | 0.02548 | 71.74 |
| 306 | 0.001398 | 0.07769 |
| 307 | 0.005996 | 20.77 |
| 308 | 0.01373 | 5.271 |
| 309 | 0.001501 | 10.62 |
| 310 | 0.0889 | 17.73 |
| 311 | 0.02446 | 15.89 |
| 312 | 0.0001563 | 0.1303 |

### Example 6. Cytokine Secretion after T Cell-Mediated Killing of CEA-Expressing Tumor Cells

The cytokine secretion of human PBMC after T cell-mediated killing of CEA-expressing HT29 tumor cells induced by the bispecific antibodies was evaluated by ELISA analysis of the cell supernatant.

The CCK8 assay was performed as described above using bispecific antibodies, using an E:T ratio of 10:1 and an incubation time of 48-72 hours. For most experiments, the incubation time was 72 hours.

After the incubation, the 96-well plate was centrifuged at 2000 rpm for 10 minutes, and the supernatant was transferred to a new 96-well plate and stored at −20° C. until subsequent analysis. R&D kits were used to detect the TNFα, IFN-γ, IL-2 and IL-6 contents of the cell supernatant on a microplate reader following manufacture instructions. The following kits were: Human IL-2 DuoSet ELISA (R&D, #DY202-05), Human IL-6 DuoSet ELISA (R&D, #DY206-05), Human IFN-γ DuoSet ELISA (R&D, #DY285B-05), Human TNF-α DuoSet ELISA (R&D, #DY210).

The ELISA detection procedure is as follows: Capture Antibody was diluted with PBS 120 times to its working concentration. 100 μl of diluted antibody was added to each well of the 96-well sample plate. The 96-well plate was sealed, and incubated overnight at room temperature. The liquid was carefully aspirated from the 96-well plate. The 96-well plate was washed three times with 300 μl Wash Buffer, and pat-dried with tissue paper several times to absorb the liquid. 300 μl of Reagent Diluent was added to each well, and the wells were sealed and incubated at room temperature for 1 h. Samples and standards were prepared during the incubation period. The standards were diluted with Reagent Diluent at a 2-fold gradient dilution, with a total of 7 dilutions and a highest concentration of 1000 μg/ml. The supernatant of the cells to be tested were diluted 2.5 times with Reagent Diluent. After blocking, the plate was washed three times with 300 μl Wash Buffer and pat-dried with tissue paper several times to absorb the liquid. 100 μl of standard or sample was added to each well and the wells were incubated at room temperature for 2 h. The wells were then washed with 300 μl Wash Buffer three times and pat-dried. Reagent Diluent was used to dilute Detection Antibody 60 times to its working concentration. 100 μl of the diluted Detection Antibody was added to each well and incubated at room temperature for 2 h. The wells were washed three times with 300 μl Wash Buffer and pat-dried. Reagent Diluent was used to dilute Streptavidin-HRP 40 times to its working concentration. 100 μl of the diluted Streptavidin-RP was added to each well and incubated at room temperature for 20 min. The wells were washed with 300 μl Wash Buffer three times and pat-dried. 100 μl Substrate Solution was added to each well. The wells were incubated in the dark at room temperature for 20 min. 50 μl Stop Solution was added to each well. The 96-well plate was tapped to mix the liquid, put into the microplate reader to read the optical density at 450 nm and 540 nm, respectively. OD450 reading value minus OD540 reading value was used as the final reading value. GraphPad software was used to fit the standard curve, and to calculate the concentration of the corresponding cytokine in the sample.

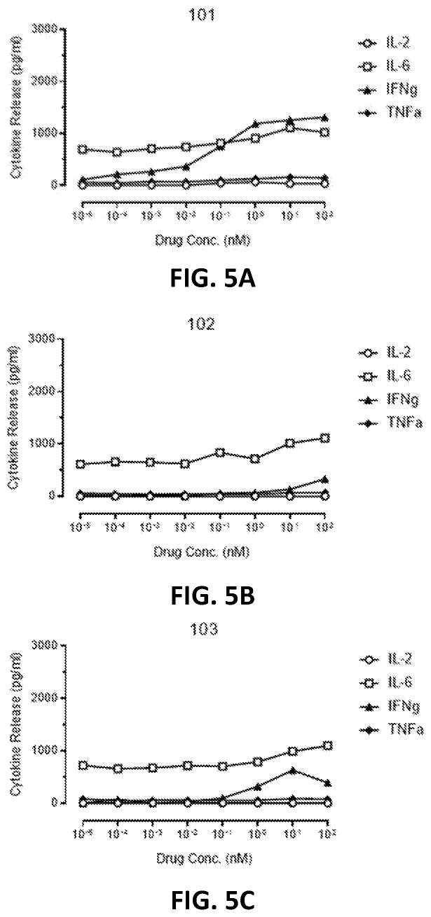

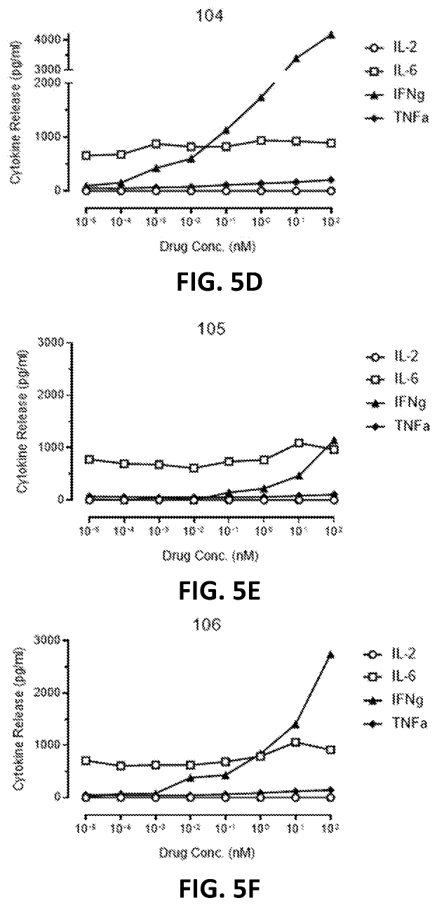

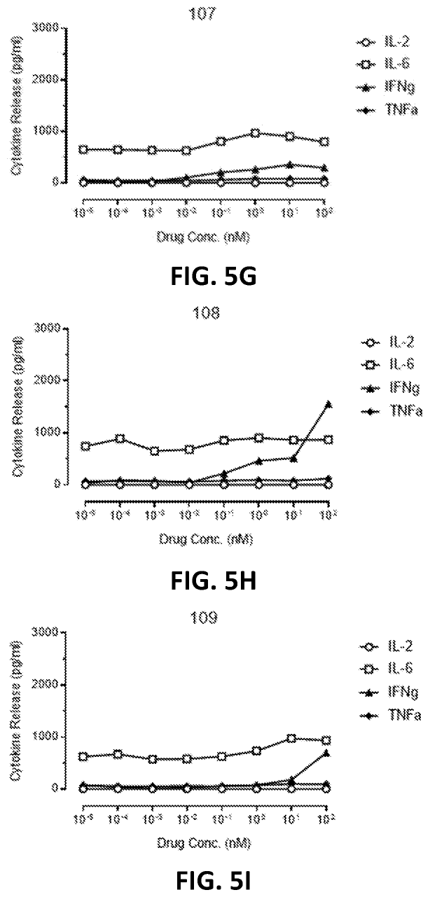

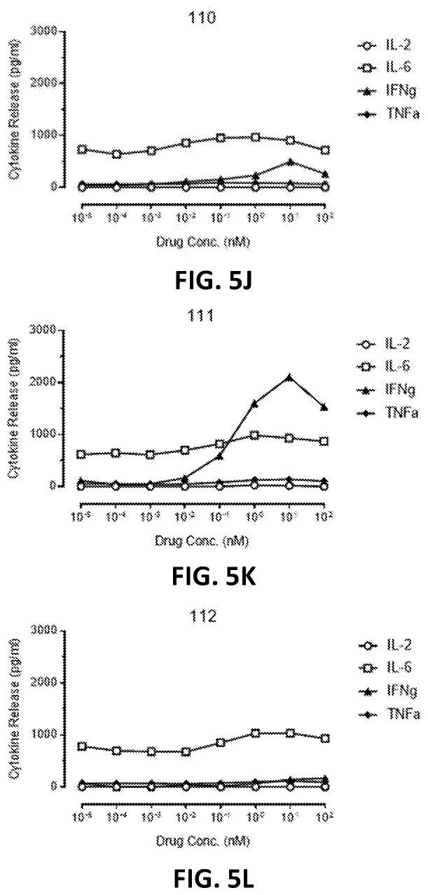

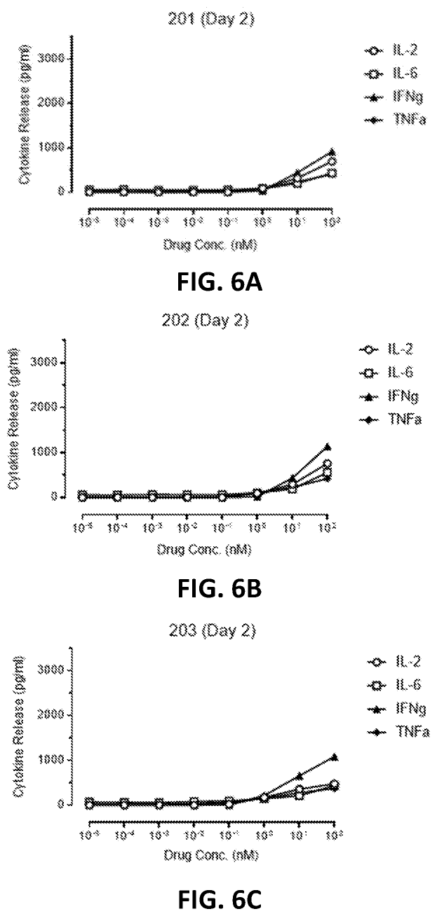

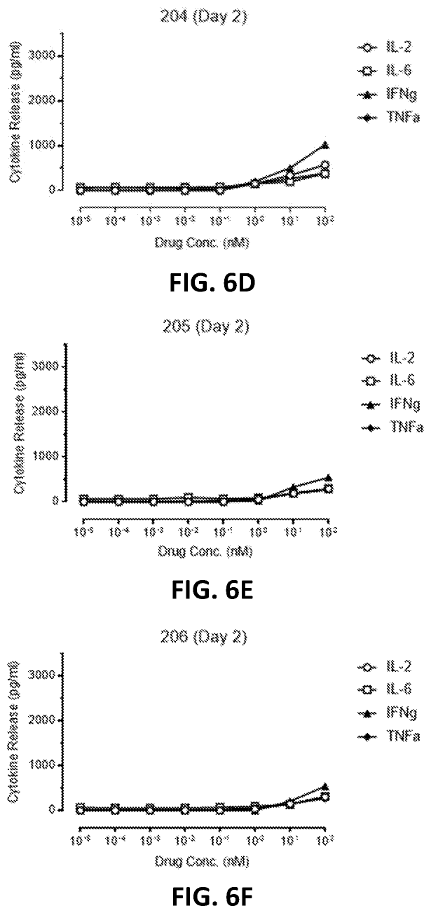

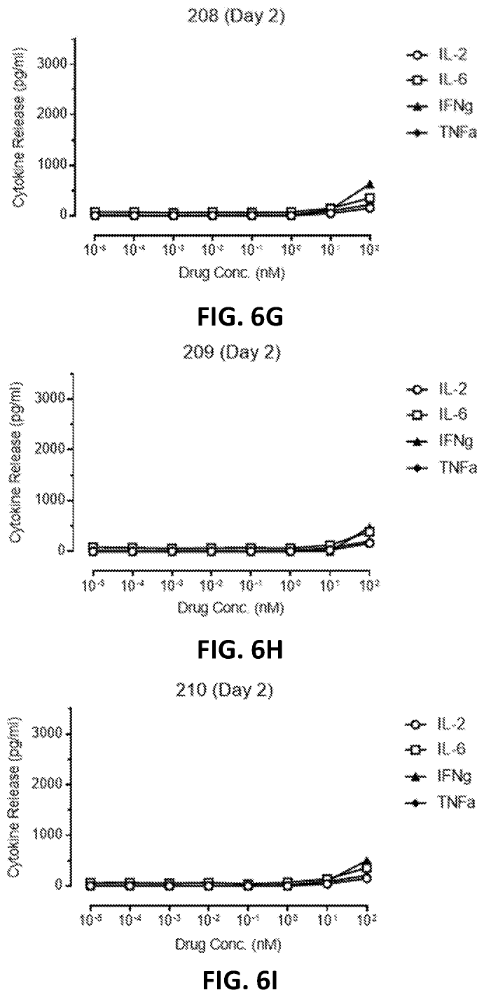

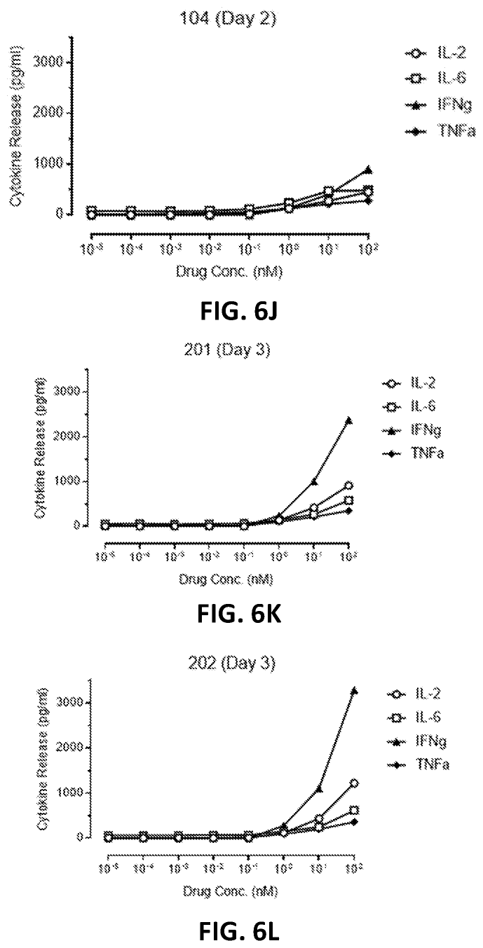

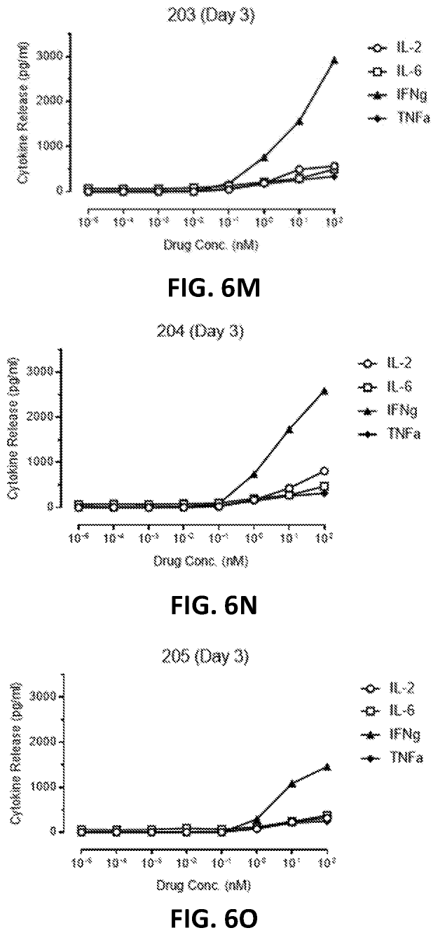

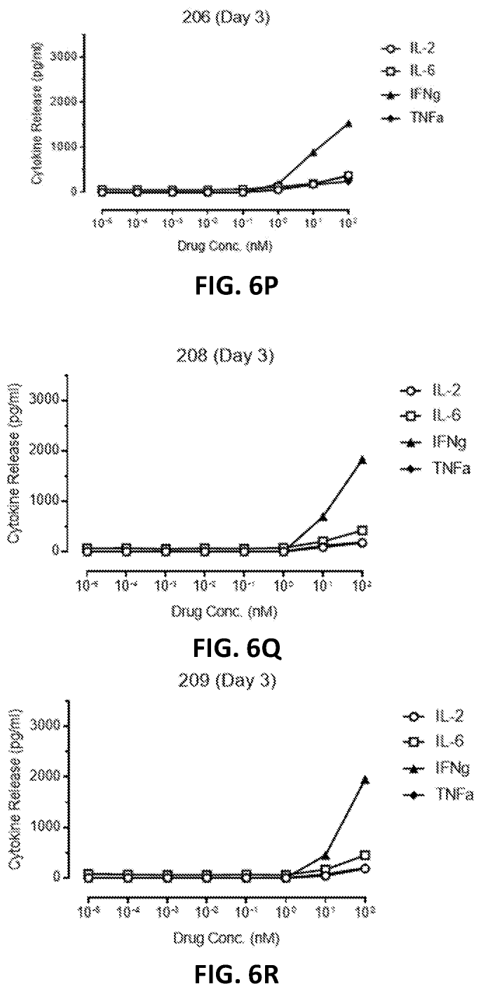

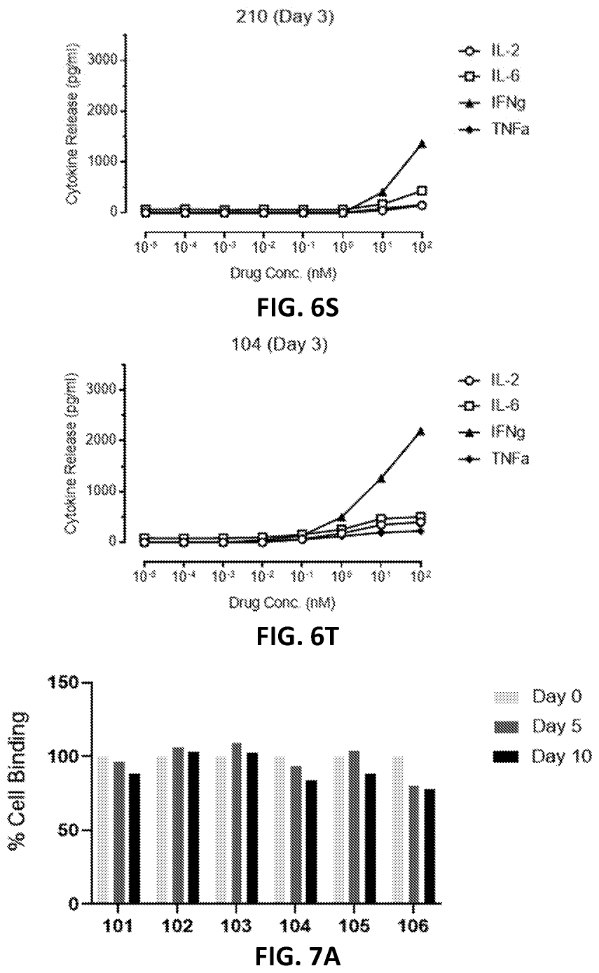

The cytokine content was first measured in the supernatant of cells incubated with the antibodies for 72 h. The bispecific antibodies significantly induced the secretion of IFN-7 during killing. IL-2, TNFα and IL-6 did not change significantly (see FIGS. 5A-5L and 6A-6T). Next, the cytokine changes were measured after 48 h and 72 h of incubation with 10 bispecific antibody molecules in real time. At 48 h, only part of the cytokines, mainly IFN-γ and IL-2, increased under the induction of high concentrations of bispecific antibodies. At 72 h, the secretion of cytokine IFN-γ, which was closely related to cell killing, significantly increased. For some bispecific antibodies at higher concentrations, the content of IL-2 and IL-6 slightly increased, indicating an increase in adverse reactions. The TNFα content did not change significantly throughout the culture cycle (see FIGS. 5A-5L and 6A-6T).

In summary, these data showed that the CEA-CD3 bispecific antibodies had excellent binding to CEA-positive H1573 cells. It induced strong target-specific killing of CEA-positive tumor cell lines without killing CEA-negative cell lines. When the cells were killed, the secretion of IFN-7 was induced, and the increase of IL-2 and IL-6 content was not obvious, indicating that the bispecific antibodies were safe to be used as therapeutic agents.

### Example 7. Thermal Stability of Bispecific Antibodies as Measured by DSF

Differential scanning fluorimetry (DSF) is a method of slowly heating a sample on a fluorescent quantitative PCR machine, and detecting the amount of fluorescent dye combined with a protein whose structure has changed during the heating process, to evaluate the thermal stability of the protein.

The thermal stability of the bispecific antibodies was monitored by DSF. After mixing 19 μl of 5 uM protein sample with 1 μl sypro orange (ThermoFisher, 56650) thoroughly, the mixture was added to a 96-well plate (Applied Biosystems, N8010560) in triplicate. After incubating at 25° C. for 30s, the temperature was increased from 25° C. to 95° C. at a rate of 0.05° C./min, and the fluorescence signal intensity was collected by a real-time fluorescent quantitative PCR instrument (Applied Biosystems, QuantStudio 5 System). PBS was used as a blank control, and Blinatumomab and Emicizumab proteins were used as positive controls. After the experiment, the Tm value was analyzed using Protein Thermal Shift software.

Tm1 was measured three times from triplicate wells. The experimental results are shown in the table below. The Tm1 value of the bispecific antibodies was 60-64.7° C. The average values of Blinatumomab and Emicizumab Tm2 were 73.5° C. and 72.4° C., respectively.

TABLE 12
Tm value of bispecific antibodies as detected by DSF

| | Tm1-1 | Tm1-2 | Tm1-3 | Average |
|---|---|---|---|---|
| Sample | (° C.) | (° C.) | (° C.) | Tm1 (° C.) |
| PBS | N/A | N/A | N/A | N/A |
| Blinatumomab | 58.2 | 58.2 | 58.2 | 58.2 |
| Emicizumab | 61.7 | 61.4 | 61.9 | 61.7 |
| 101 | 63.0 | 62.8 | 62.7 | 62.8 |
| 102 | 62.0 | 62.1 | 62.3 | 62.1 |
| 103 | 62.5 | 62.6 | 62.4 | 62.5 |
| 104 | 62.4 | 62.5 | 62.6 | 62.5 |
| 105 | 63.0 | 62.8 | 62.8 | 62.9 |
| 106 | 62.3 | 62.1 | 62.4 | 62.3 |
| 107 | 61.0 | 60.9 | 60.9 | 60.9 |
| 108 | 61.3 | 61.1 | 60.9 | 61.1 |
| 109 | 60.6 | 60.3 | 60.3 | 60.4 |
| 110 | 61.2 | 61.2 | 61.3 | 61.2 |
| 111 | 61.6 | 61.6 | 61.7 | 61.6 |
| 112 | 60.9 | 60.9 | 60.9 | 60.9 |
| 201 | 62.6 | 62.5 | 62.4 | 62.5 |
| 202 | 62.6 | 62.6 | 62.5 | 62.6 |
| 203 | 61.7 | 62.1 | 61.7 | 61.9 |
| 204 | 61.8 | 62.0 | 62.2 | 62.0 |
| 205 | 61.9 | 61.7 | 61.7 | 61.7 |
| 206 | 62.2 | 62.2 | 62.1 | 62.1 |
| 208 | 62.7 | 62.7 | 62.4 | 62.6 |
| 209 | 62.3 | 62.1 | 62.2 | 62.2 |
| 210 | 61.2 | 61.1 | 61.1 | 61.1 |
| 301 | 63.9 | 63.8 | 63.8 | 63.9 |
| 302 | 64.1 | 64.1 | 64.0 | 64.1 |
| 303 | 64.0 | 64.1 | 64.1 | 64.0 |
| 304 | 64.3 | 64.4 | 64.4 | 64.3 |
| 305 | 64.5 | 64.4 | 64.4 | 64.4 |
| 306 | 64.4 | 64.4 | 64.3 | 64.3 |
| 307 | 63.8 | 63.8 | 63.6 | 63.7 |
| 308 | 64.1 | 64.1 | 64.2 | 64.2 |
| 309 | 64.2 | 64.1 | 64.1 | 64.1 |
| 310 | 64.1 | 64.2 | 64.1 | 64.1 |
| 311 | 64.1 | 64.2 | 64.1 | 64.1 |
| 312 | 64.1 | 64.3 | 64.2 | 64.2 |

### Example 8. Serum Stability of Bispecific Antibodies as Measured by Flow Cytometry (FACS)

The samples to be tested were centrifuged at 4° C. at 12000 rpm for 30 minutes to remove the precipitate. 200 μl of each sample to be tested (0.5 mg/ml) was collected in a 1.5 ml Eppendorf tube. An equal volume of human serum (GEMINI, H2OYOOK) was added, mixed thoroughly, and the mixture was incubated in a 37° C. constant temperature CO₂ incubator. For 10 days, 40 μl samples were aspirated every day and stored at −20° C.

Flow cytometry was used to detect the binding of the bispecific antibodies to the human lung adenocarcinoma cell line (H1395) with high CEA expression. The procedure is as follows: the cells were harvested, counted, and resuspended in FACS buffer (PBS containing 0.1% BSA) at 2×10⁶ cells/ml. 100 μl of cell suspension was incubated in a V-bottom 96-well plate at 4° C. with a final concentration of 10 μg/ml bispecific antibodies for 30 minutes. The cells were washed twice with pre-cooled FACS buffer. The PE-conjugated goat anti-human IgG Fc fragment-specific secondary antibody (eBioscience, 12-4998-82) was incubated at 4° C. for 30 minutes. The cells were washed twice with pre-cooled FACS buffer, and analyzed immediately with a flow cytometer (Beckman Coulter, Cytoflex).

The binding of the bispecific antibodies was judged by the change in fluorescence value, where Cell Binding (%)=[(F_{dayn}−F_{b})/(F_{day0}−F_{b})]×100%. F_{dayn}: experimental well (protein was stored in serum for n days); F_{day0}: control well (protein was not stored in serum); F_{b}: blank well (cells without primary antibody).

Experimental results showed that most bispecific antibodies have good serum stability. For example, when protein 101 and 104 were placed in the serum for up to ten days, the binding to CEA-positive target cells was still maintained at more than 80%.

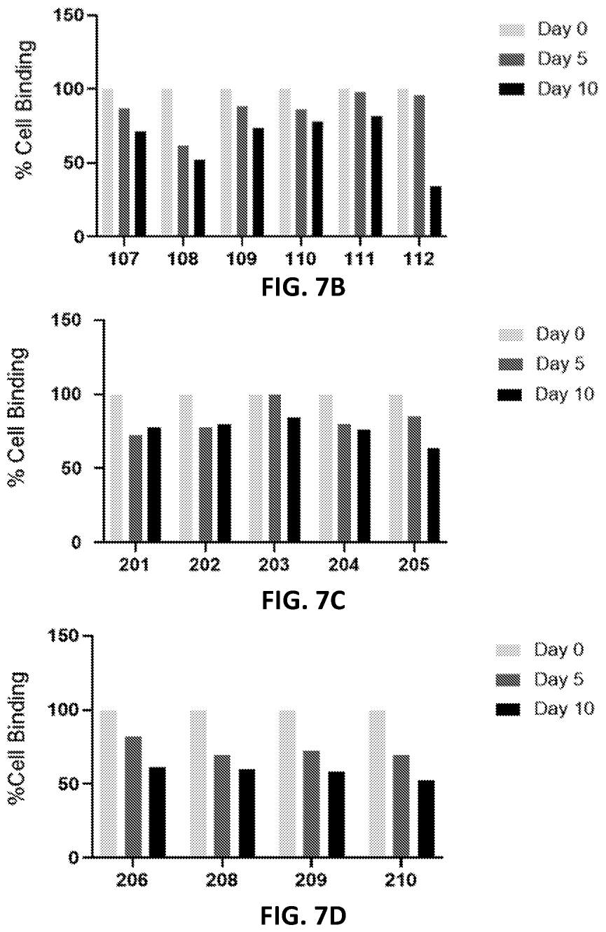

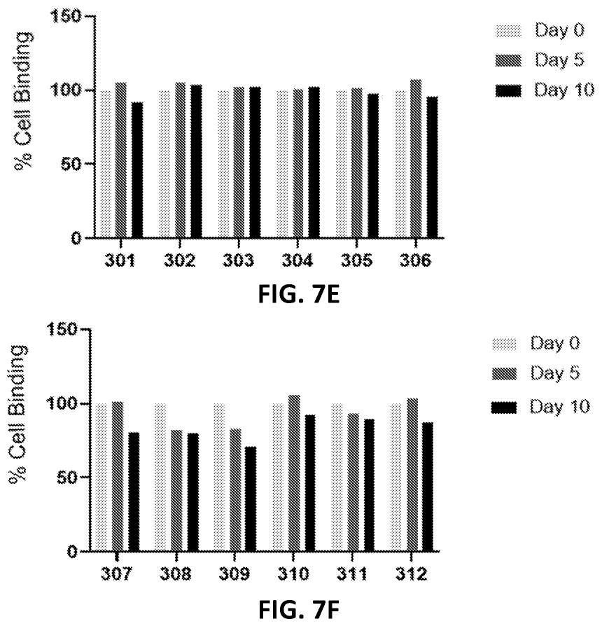

The results are shown in FIGS. 7A-7F and the table below.

TABLE 13
Serum stability of bispecific antibodies as detected by FACS

| | % Cell Binding (H1395)/Day 0 | | | | | | | | | | |
|---|---|---|---|---|---|---|---|---|---|---|---|
| Sample | Day 0 | Day 5 | Day 10 | Sample | Day 0 | Day 5 | Day 10 | Sample | Day 0 | Day 5 | Day 10 |
| 101 | 100 | 96.75 | 88.14 | 201 | 100 | 72.82 | 77.30 | 301 | 100 | 104.88 | 91.80 |
| 102 | 100 | 105.85 | 102.93 | 202 | 100 | 77.93 | 79.75 | 302 | 100 | 104.92 | 103.40 |
| 103 | 100 | 109.11 | 102.25 | 203 | 100 | 99.85 | 84.44 | 303 | 100 | 102.19 | 101.71 |
| 104 | 100 | 93.67 | 83.52 | 204 | 100 | 80.14 | 76.47 | 304 | 100 | 100.29 | 101.81 |
| 105 | 100 | 103.83 | 88.17 | 205 | 100 | 85.19 | 63.79 | 305 | 100 | 100.96 | 97.19 |
| 106 | 100 | 80.32 | 78.36 | 206 | 100 | 82.25 | 61.21 | 306 | 100 | 107.15 | 95.04 |
| 107 | 100 | 86.68 | 71.40 | 208 | 100 | 69.27 | 59.83 | 307 | 100 | 101.32 | 80.85 |
| 108 | 100 | 61.68 | 52.38 | 209 | 100 | 72.36 | 58.22 | 308 | 100 | 82.25 | 79.52 |
| 109 | 100 | 88.44 | 73.51 | 210 | 100 | 69.66 | 52.26 | 309 | 100 | 83.12 | 71.05 |
| 110 | 100 | 86.40 | 78.08 | | | | | 310 | 100 | 105.43 | 92.18 |
| 111 | 100 | 98.05 | 81.67 | | | | | 311 | 100 | 93.17 | 89.66 |
| 112 | 100 | 95.67 | 34.36 | | | | | 312 | 100 | 103.64 | 87.34 |

### Example 9. Detection of Serum Stability of Bispecific Antibodies by ELISA

The samples to be tested were centrifuged at 4° C. at 12000 rpm for 30 minutes to remove the precipitate. 200 μl of each sample to be tested (0.5 mg/ml) was collected in a 1.5 ml Eppendorf tube. An equal volume of human serum (GEMINI, H2OYOOK) was added, mixed thoroughly, and the mixture was incubated in a 37° C. constant temperature CO₂ incubator. For a total of 10 days, 40 μl samples was aspirated every day and stored at −20° C.

Enzyme-linked immunosorbent assay (ELISA) was used to test the bispecific antibodies' binding to human CEA and human CD3 epsilon/delta antigen. The steps are as follows: the antigen human CD3ε/δ (ACRO systems, CDD-H52W1) and the recombinant tumor antigen human CEA (CEACAM-5/CD66e, ACRO systems, CE5-H5226) were diluted to 1 μg/ml with PBS, and then added to the 96-well plate at 100 μl per well. The wells were coated overnight at 4° C. After washing 3 times with 0.05% PBST, 200 μl 5% skimmed milk powder was added to each well and incubated at 37° C. for 2 h. After washing 3 times with 0.05% PBST, 100 μl primary antibody sample (10 μg/ml) was added to each well, and incubated at 37° C. for 2 h. After washing 3 times with 0.05% PBST, 100 μl (1:6000) diluted secondary antibody goat anti-human Fc-HRP (sigma, A2170) was added to each well, incubated at room temperature for 1 h. After washing 8 times with 0.05% PBST, 50 μl TMB color developing solution was added to each well, and incubated at room temperature for 10-15 min. 50 μl 1 mM HCl was added to each well to stop the color development. 450 nm Absorbance (OD) was read.

The binding of antibodies to antigen was calculated according to the following formula: Binding (%)=[(OD_{dayn}−OD_{b})/(OD_{day0}−OD_{b})]×100%. OD_{dayn}: experimental well (the protein was stored in the serum for n days); OD_{day0}: control well (the protein was not stored in the serum); OD_{b}: blank well (no primary antibody added).

The experimental results show that the bispecific antibodies have good serum stability. After storing in serum for up to ten days, the antibodies still maintain strong binding to the antigens CEA and CD3. The experimental results are shown in the tables below.

TABLE 14
The serum stability of bispecific antibodies (101-112) as measured by ELISA

| Day | 101 | 102 | 103 | 104 | 105 | 106 | 107 | 108 | 109 | 110 | 111 | 112 |
|---|---|---|---|---|---|---|---|---|---|---|---|---|
| | CEA (Binding %) | | | | | | | | | | | |
| Day 0 | 100 | 100 | 100 | 100 | 100 | 100 | 100 | 100 | 100 | 100 | 100 | 100 |
| Day 1 | 99.09 | 104.75 | 104.66 | 99.2 | 101.63 | 98.01 | 98.7 | 98.51 | 99.11 | 98.06 | 99.55 | 107.73 |
| Day 2 | 100.68 | 98.42 | 98.6 | 97.37 | 100.5 | 102.57 | 94.91 | 97.37 | 105.46 | 101.71 | 99.55 | 105.74 |
| Day 5 | 102.27 | 103.33 | 100 | 97.14 | 104.27 | 101.52 | 100.36 | 99.09 | 104.45 | 100.34 | 102.27 | 101.87 |
| Day 6 | 100.68 | 101.15 | 105.36 | 96.45 | 107.79 | 105.37 | 102.49 | 104.57 | 102.67 | 102.63 | 99.66 | 97.76 |
| Day 7 | 105 | 106.28 | 109.43 | 105.61 | 109.55 | 110.86 | 104.38 | 105.03 | 110.8 | 102.05 | 107.14 | 101.87 |
| Day 9 | 101.82 | 109.67 | 107.45 | 107.78 | 101.56 | 106.66 | 109.24 | 109.03 | 101.69 | 103.77 | 106.24 | 102.37 |
| Day 10 | 107.16 | 108.2 | 110.94 | 110.87 | 104.82 | 110.98 | 111.49 | 110.51 | 103.85 | 107.31 | 106.8 | 101 |
| | CD3 ε/δ (Binding %) | | | | | | | | | | | |
| Day 0 | 100 | 100 | 100 | 100 | 100 | 100 | 100 | 100 | 100 | 100 | 100 | 100 |
| Day 1 | 98.45 | 105.76 | 101.13 | 107.21 | 102.6 | 110.64 | 117.93 | 93.01 | 97.45 | 68.48 | 93.21 | 99.02 |
| Day 2 | 101.33 | 110.94 | 105.75 | 109.61 | 105.67 | 115.24 | 123.42 | 97.98 | 88.5 | 74.33 | 99.35 | 102.01 |
| Day 5 | 99.22 | 108 | 104.51 | 103.53 | 106.74 | 116.29 | 121.65 | 96.1 | 88.04 | 78.06 | 102.87 | 103.55 |
| Day 6 | 102.66 | 109.88 | 105.07 | 104.16 | 104.02 | 121.68 | 122.78 | 98.39 | 85.65 | 76.89 | 99.22 | 109.63 |
| Day 7 | 100.44 | 105.29 | 106.76 | 103.02 | 101.33 | 118.27 | 125.2 | 98.79 | 87.59 | 77.53 | 105.87 | 103.55 |
| Day 9 | 101.77 | 100.12 | 103.38 | 108.47 | 104.61 | 112.09 | 117.77 | 98.52 | 83.26 | 77 | 96.48 | 102.38 |
| Day 10 | 100.78 | 101.71 | 100.34 | 107.08 | 99.17 | 106.31 | 102.1 | 66.67 | 62.07 | 56.12 | 78.2 | 98.36 |

TABLE 15
The serum stability of bispecific antibodies (201-210) as measured by ELISA

| Day | 201 | 202 | 203 | 204 | 205 | 206 | 208 | 209 | 210 |
|---|---|---|---|---|---|---|---|---|---|
| | CEA (Binding %) | | | | | | | | |
| Day 0 | 100 | 100 | 100 | 100 | 100 | 100 | 100 | 100 | 100 |
| Day 1 | 99.44 | 101.11 | 102.34 | 105.15 | 107.5 | 98.12 | 99.44 | 103.3 | 100.55 |
| Day 2 | 98.87 | 99.67 | 95.53 | 100.9 | 105.45 | 96.55 | 99.32 | 101.59 | 97.89 |
| Day 5 | 100.45 | 96.99 | 94.78 | 92.95 | 100.57 | 91.32 | 95.04 | 98.3 | 93.9 |
| Day 6 | 94.59 | 95.66 | 95.21 | 96.98 | 101.48 | 90.06 | 97.29 | 96.59 | 99.45 |
| Day 7 | 99.44 | 100.78 | 95.74 | 97.2 | 98.98 | 91.84 | 95.94 | 96.36 | 94.9 |
| Day 9 | 98.42 | 96.88 | 94.57 | 99.66 | 102.27 | 92.36 | 93.91 | 100.8 | 100.22 |
| Day 10 | 101.69 | 100.33 | 100.21 | 98.66 | 104.77 | 95.61 | 99.77 | 103.86 | 101.88 |
| | CD3 ε/δ (Binding %) | | | | | | | | |
| Day 0 | 100 | 100 | 100 | 100 | 100 | 100 | 100 | 100 | 100 |
| Dav 1 | 104.18 | 108.5 | 102.79 | 105.22 | 96.46 | 95.13 | 95.7 | 101.85 | 104.68 |
| Day 2 | 76.3 | 86.78 | 85.3 | 101.51 | 99.27 | 95.13 | 96.11 | 102.84 | 101.7 |
| Day 5 | 104.63 | 110.04 | 102.15 | 99.77 | 117.2 | 102.16 | 100 | 108.18 | 97.34 |
| Day 6 | 101.58 | 107.91 | 100.97 | 102.9 | 100.24 | 99.35 | 97.44 | 104.91 | 101.38 |
| Day 7 | 104.4 | 107.56 | 105.58 | 103.13 | 104.76 | 100.22 | 95.91 | 108.83 | 101.7 |
| Day 9 | 101.35 | 104.13 | 102.58 | 104.76 | 104.51 | 95.56 | 98.77 | 108.4 | 101.17 |
| Day 10 | 98.76 | 99.06 | 100.97 | 99.42 | 105.37 | 91.56 | 95.7 | 107.09 | 100.96 |

TABLE 16
The serum stability of bispecific antibodies (301-312) as measured by ELISA

| Day | 301 | 302 | 303 | 304 | 305 | 306 | 307 | 308 | 309 | 310 | 311 | 312 |
|---|---|---|---|---|---|---|---|---|---|---|---|---|
| | CEA (Binding %) | | | | | | | | | | | |
| Day 0 | 100 | 100 | 100 | 100 | 100 | 100 | 100 | 100 | 100 | 100 | 100 | 100 |
| Day 1 | 97.85 | 102.12 | 101.13 | 99.79 | 97.39 | 99.12 | 102.58 | 97.98 | 96.91 | 99.79 | 101.02 | 97.35 |
| Day 2 | 99.46 | 106.16 | 102.88 | 101.26 | 99.9 | 108.41 | 99.78 | 104.15 | 103.7 | 104.81 | 94.26 | 103.53 |
| Day 5 | 95.17 | 108.88 | 106.28 | 102.83 | 106.89 | 109.07 | 108.28 | 106.06 | 103.4 | 106.48 | 100.92 | 104.71 |
| Day 6 | 101.5 | 111 | 108.75 | 108.48 | 108.56 | 101.62 | 111.83 | 100.64 | 105.56 | 109.51 | 106.76 | 107.94 |
| Day 7 | 96.78 | 111.3 | 104.62 | 100.16 | 102.21 | 100.84 | 102.58 | 100.43 | 108.54 | 104.21 | 109.43 | 109.8 |
| Day 9 | 99.79 | 103.82 | 104.73 | 103.09 | 102.53 | 106.26 | 104.09 | 102.87 | 109.67 | 103.38 | 101.48 | 102.25 |
| Day 10 | 103.86 | 108.97 | 109.57 | 108.95 | 107.43 | 105.88 | 100.97 | 108.83 | 104.4 | 100.27 | 105.78 | 105.78 |
| | CD3 ε/δ (Binding %) | | | | | | | | | | | |
| Day 0 | 100 | 100 | 100 | 100 | 100 | 100 | 100 | 100 | 100 | 100 | 100 | 100 |
| Day 1 | 107.98 | 108.17 | 104.83 | 108.34 | 111.89 | 111.99 | 107.15 | 106.16 | 94.19 | 96.08 | 105.29 | 103.47 |
| Day 2 | 94.09 | 104.24 | 108.41 | 106.29 | 109.05 | 109.7 | 104.56 | 100.1 | 86.8 | 90.21 | 104.66 | 106.13 |
| Day 5 | 110.45 | 110.44 | 108.97 | 100.19 | 110.21 | 114.7 | 107.03 | 106.87 | 104.65 | 92.16 | 103.19 | 103.73 |
| Day 6 | 109.08 | 107.14 | 103.85 | 104.54 | 110.53 | 101.36 | 103.08 | 103.42 | 100.84 | 93.2 | 104.35 | 101.51 |
| Day 7 | 100.83 | 104.34 | 103.43 | 107.27 | 109.68 | 105.21 | 101.23 | 109.02 | 96.3 | 89.18 | 101.41 | 98.22 |
| Day 9 | 95.87 | 103.1 | 105.52 | 101.17 | 109.05 | 95.1 | 98.64 | 109.62 | 89.23 | 90.31 | 100.37 | 100.89 |
| Day 10 | 69.6 | 106 | 103.77 | 101.35 | 108.21 | 111.68 | 87.79 | 102.6 | 93.56 | 100.82 | 101.31 | 104 |

### Example 10: Preparation of EGFR-CD3 Bispecific Antibodies and HER2-CD3 Bispecific Antibodies Using TA1 as the Anti-CD3 Antibody

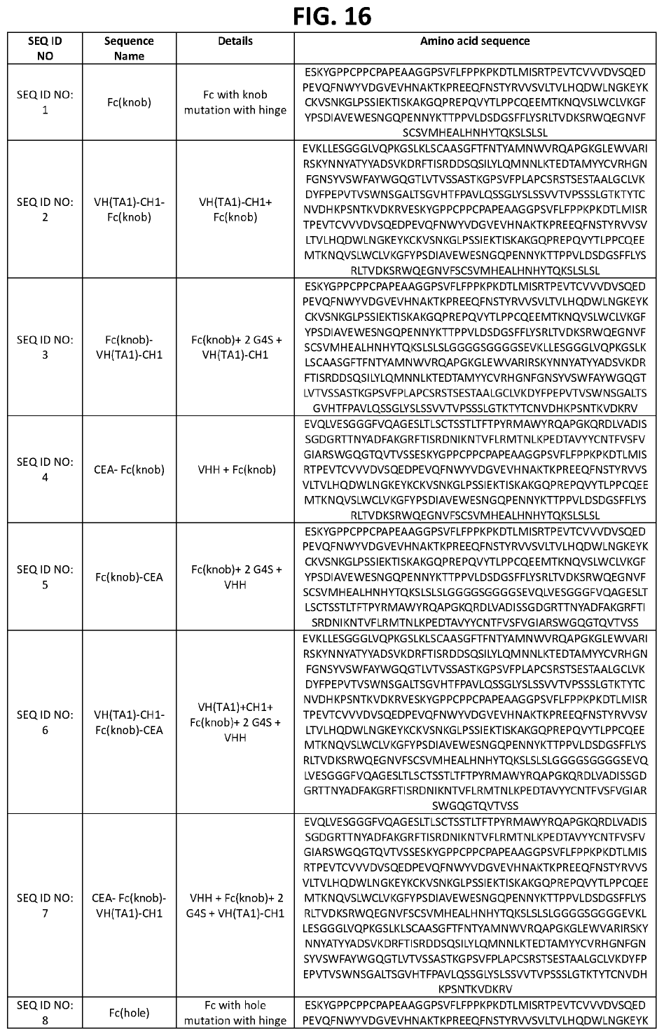

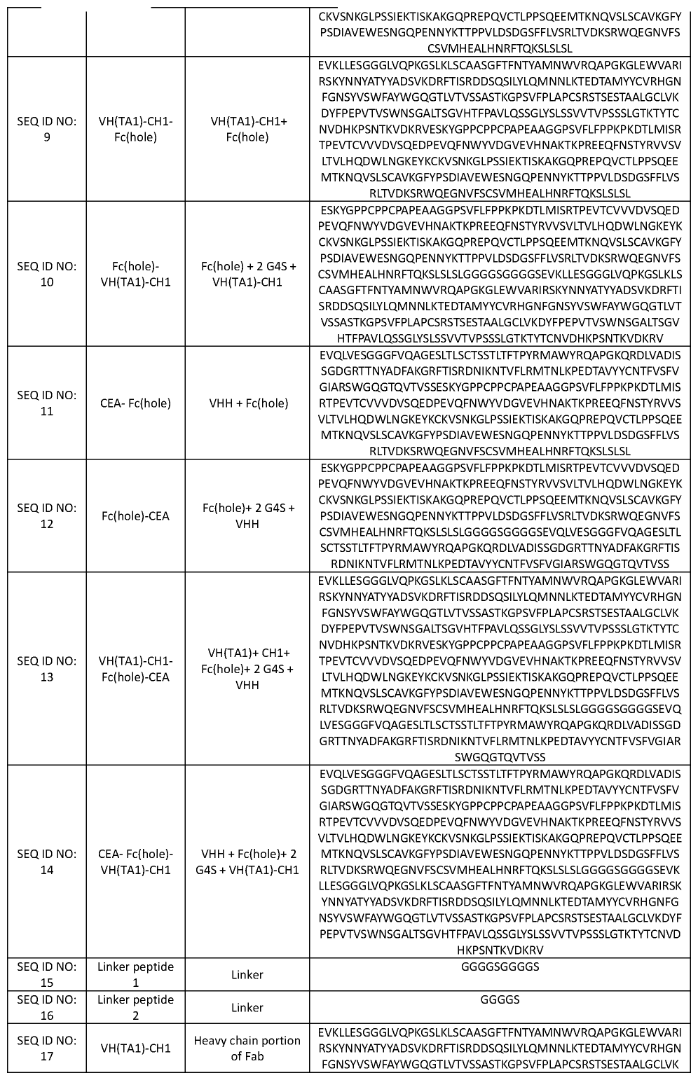

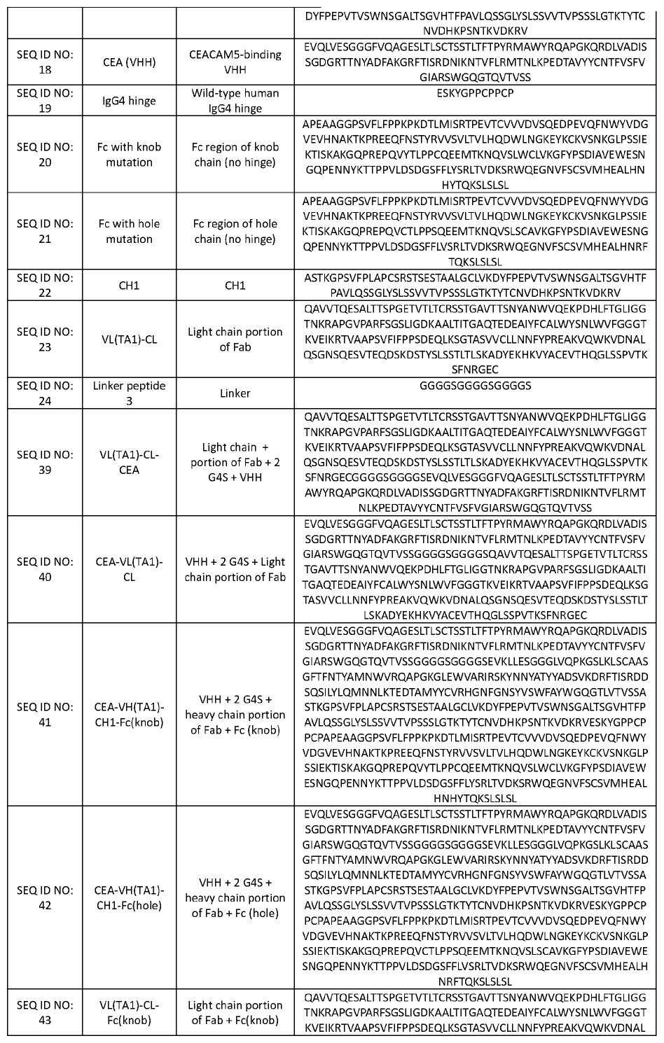

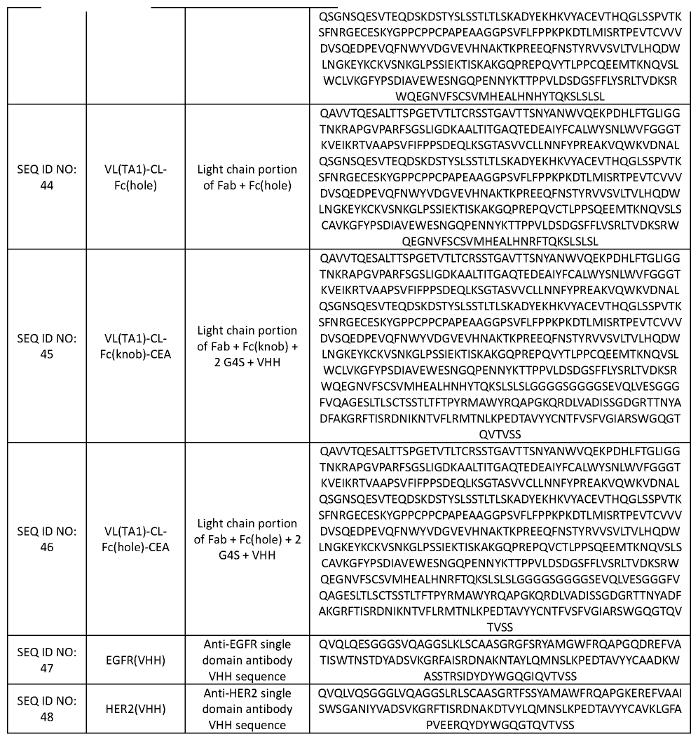

According to the structures of 101 and 104 in FIG. 1, two EGFR-CD3 bispecific antibodies (401 and 404) and two HER2-CD3 bispecific antibodies (501 and 504) were constructed using TA1 as the CD3 antibody. The anti-EGFR single domain antibody VHHT sequence (EGFR(VHH)) and anti-HER2 single domain antibody VHHT sequence (HER2(VHH)) are shown in FIG. 16 as SEQ TD NO: 47 and SEQ TD NO: 48, respectively.

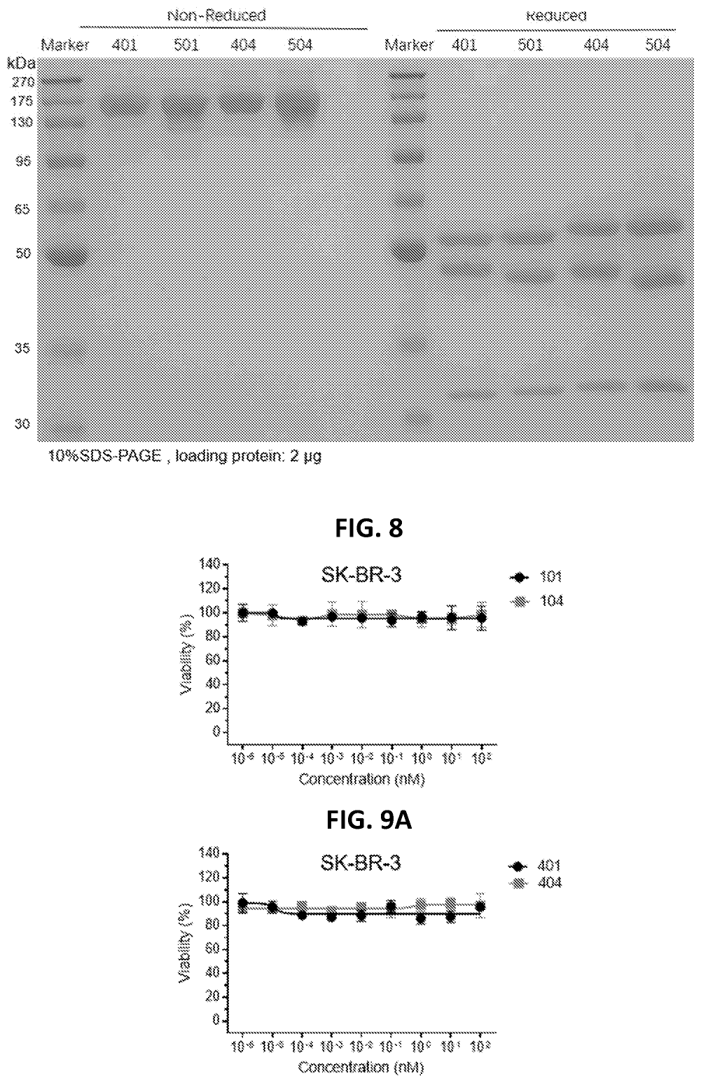

The protein structure design of the bispecific antibodies is shown in FIG. 1A (101) and 1D (104), where the anti-CEA single domain antibody (CEA) was replaced with an anti-EGFR single domain antibody (EGFR(VHH)) or an anti-HER2 single domain antibody (HER2(VHH)). The SDS-PAGE results are shown in FIG. 8. The protein expression and purification method is the same as before, and the yield and purity results are shown in the table below.

TABLE 17
Yield and purity of the EGFR-CD3 and
HER2-CD3 bispecific antibodies

| | Yield | |
|---|---|---|
| Sample | (mg/L) | Purity % |
| 401 | 28.0 | 95.45 |
| 501 | 19.7 | 92.82 |
| 404 | 23.6 | 95.55 |
| 504 | 21.3 | 93.93 |

### Example 11: Determining the Affinity of EGFR-CD3 and HER2-CD3 Bispecific Antibodies for Human EGFR, HER2 and Human CD3ε/δ Through BLI

Biofilm interferometry (BLI) assays were performed on OCTECT RED96e (ForteBio) at 30° C. with 0.02% PBST solution as running buffer (10 mmol/L Na2HPO4; 1.75 mmol/L KH2PO4; 137 mmol/L NaCl; 2.65 mmol/L KCl; pH 7.2-7.4, 0.02% Surfactant Tween 20).

The bispecific antibodies (401, 404, 501 and 504) and the control antibodies were first captured on the chip surface of an AHC sensor (Anti-hIgG Fc, sartorius, 18-5060) with immobilized anti-human Fc antibodies. Bispecific antibodies and EGFR mAb control Cetuximab (Wuhan Chemstan Biotechnology, CSD00079) and HER2 mAb control Trastuzumab (Wuhan Chemstan Biotechnology, CSAD00686) were diluted to 5 μg/ml, and 1.5 nm of each protein was coupled to the AHC sensor chip surface in 0.02% PBST.

As the analytes, human CD3ε/δ (ACRO systems, CDD-H52W1), recombinant tumor antigen human EGFR (ACRO systems, EGR-H5222) and recombinant tumor antigen human HER2 (ERBB2, ACRO systems, HE2-H5212) were diluted in 0.02% PBST solution to 200 nM, 100 nM, 50 nM, 25 nM, 12.5 nM, 6.25 nM, and 3.125 nM.

After the chip captures the bispecific antibodies, it was allowed to bind and dissociate with different concentrations of analytes to obtain the KD value of the interaction. The experimental parameters are as follows: Baseline1: 60s, Loading: 240s, Baseline2: 180s, Association: 240s, Dissociation: 600s, Loading Response: 1.9-2.1 (nm), High sensitivity kinetics: 2 Hz. Kinetic constants were derived based on 1:1 Langmuir binding to obtain equilibrium dissociation constants (KD). Date Analysis HT 12 software (Sartorius, 50-5029) was used to simultaneously calculate Kₒₙ, K_{off}, and the affinity (KD=K_{off}/Kₒₙ), using the binding dissociation curve.

The experimental results showed that the affinity of the bispecific antibodies to CD3 was 2.65 nM-3.71 nM. The affinity of the bispecific antibodies to EGFR was about 2 nM, close to the affinity of Cetuximab (1.33 nM). The affinity of the bispecific antibodies to HER2 was 13.1 nM-18.0 nM, far lower than Trastuzumab's Affinity (0.3 nM). The affinity KD data are shown in the tables below.

TABLE 18
Affinities of bispecific antibodies to human CD3 ε/δ (T = 30° C.)
Human CD3ε/CD3δ

| Sample | kₐ (1/Ms) | k_{dis} (1/s) | KD (M) |
|---|---|---|---|
| 401 | 1.81E+05 | 4.91E−04 | 2.71E−09 |
| 501 | 1.62E+05 | 5.08E−04 | 3.14E−09 |
| 404 | 1.77E+05 | 4.69E−04 | 2.65E−09 |
| 504 | 1.79E+05 | 5.00E−04 | 2.79E−09 |

TABLE 19
Affinities of bispecific antibodies to human EGFR (T = 30° C.)
Human EGFR

| Sample | kₐ (1/Ms) | k_{dis} (1/s) | KD (M) |
|---|---|---|---|
| Cetuximab | 1.18E+06 | 1.57E−03 | 1.33E−09 |
| 401 | 9.69E+05 | 2.01E−03 | 2.07E−09 |
| 404 | 1.08E+06 | 2.38E−03 | 2.20E−09 |

TABLE 20
Affinities of bispecific antibodies to human HER2 (T = 30° C.)
Human HER2

| Sample | kₐ (1/Ms) | k_{dis} (1/s) | KD (M) |
|---|---|---|---|
| Trastuzumab | 4.96E+05 | 1.49E−04 | 3.00E−10 |
| 501 | 2.66E+05 | 3.49E−03 | 1.31E−08 |
| 504 | 2.30E+05 | 4.13E−03 | 1.80E−08 |

### Example 12: T Cell Mediated Killing of EGFR/HER2 Expressing Tumor Target Cells Induced by EGFR-CD3/HER2-CD3 Bispecific Antibodies

To assess EGFR-targeting EGFR-CD3 bispecific antibodies (401 and 404), the following tumor cell lines were used: (1) human breast adenocarcinoma cell line MDA-MB-468, which expresses higher levels of EGFR; (2) human non-small cell lung cancer cell line NCI-H1650, which expresses high levels of EGFR; (3) human lung papillary adenocarcinoma cell line NCI-H441, which expresses high levels of EGFR; (4) human non-small cell lung cancer cell line NCI-H1573, which expresses high levels of EGFR; (5) human ovarian cancer cell line SKOV-3, which expresses high levels of EGFR; (6) human gastric cancer cell line N87, which expresses low levels of EGFR; (7) human lung adenocarcinoma cell line NCI-H2228, which expresses moderate levels of EGFR; (8) human breast cancer cell line MCF-7, which expresses moderate levels of EGFR; (9) human lung squamous cell carcinoma cell NCI-H157, which expresses low levels of EGFR; (10) human breast adenocarcinoma cell line SK-BR-3, which does not express EGFR; (11) human breast cancer cell line MDA-MB-453, which does not express EGFR; (12) human lung squamous cell carcinoma cell NCI-H157 clone 21 (NCI-H157-21 #), which is a monoclonal cell line with EGFR knockdown (iGene Biotechnology, HSH117865-LVRU6GP).

To assess HER2-targeting HER2-CD3 bispecific antibodies (501 and 504), the following tumor cell lines were used: (1) human non-small cell lung cancer cell line NCI-H1573, which expresses high levels of HER2; (2) human ovarian cancer cell line SKOV-3, which expresses high levels of HER2; (3) human lung papillary adenocarcinoma cell line NCI-H441, which expresses moderate levels of HER2; (4) human gastric cancer cell line N87, which expresses moderate levels of HER2; (5) human breast cancer cell line MDA-MB-453, which expresses low levels equal levels of HER2; (6) human breast cancer cell line MDA-MB-468, which expresses low levels of HER2; (7) human non-small cell lung cancer cell line NCI-H1650, which expresses low levels of HER2; (8) human lung adenocarcinoma cell line NCI-H2228, which expresses low levels of HER2; (9) human breast cancer cell line MCF-7, (10) human lung squamous cell carcinoma cell NCI-H157, which express low level of HER2; (11) human breast adenocarcinoma cell line SK-BR-3, which is HER2 negative.

T cell mediated killing was detected 72 hours after incubation with bispecific antibodies using human PBMCs as effector cells. Tumor cells were digested with trypsin/EDTA, washed once with pre-cooled PBS, re-suspended in RPMI1640 medium containing 10% FBS, and plated in a flat-bottom 96-well plate (Corning 3599) at a density of 5,000 cells/well. After incubating for 4 hours, 50 μL of serially diluted bispecific antibody solution was added to each well, with 3 duplicate wells for each concentration. The bispecific antibodies were incubated with the cells for 30 min, so that the bispecific antibodies are fully attached to the cells. PBMC cells (Reid Bio, 1521) were resuscitated after rapid thawing, added to 10 mL of RPMI 1640 medium containing 10% FBS, centrifuged at 1000 rpm for 5 min, and re-suspended in the complete medium after discarding the supernatant. Cell density was adjusted as needed, and 50 μL of PBMCs were added to each well of target cells to reach a 10:1 E:T ratio. The 96-well plate was placed in a 37° C., 5% CO₂ incubator for 3 days.

After three days of culturing, the cell culture supernatant was carefully removed. The 96-well plate was pat dried with paper towel, and Serum-Free DMEM containing 10% CCK8 detection solution (Dojindo, CK04-20) was added to the 96-well plate, and incubated at 37° C. for 2-4 h. The absorbance at 450 nm was detected on a microplate reader (BioTech, Synergy LX), and the cell viability was calculated.

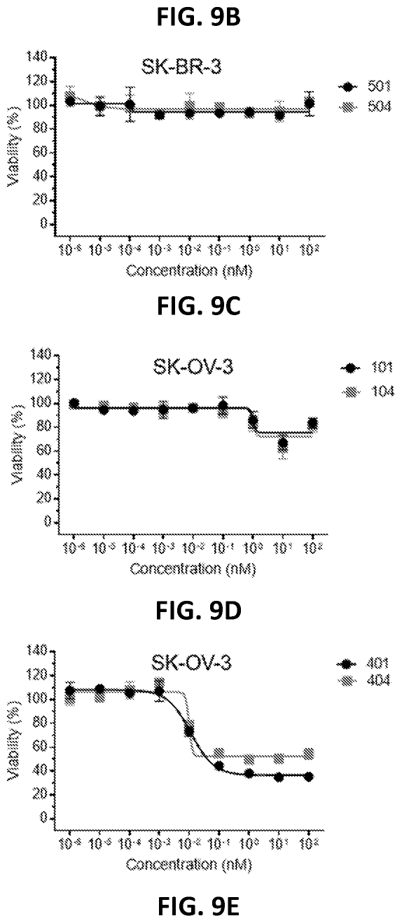

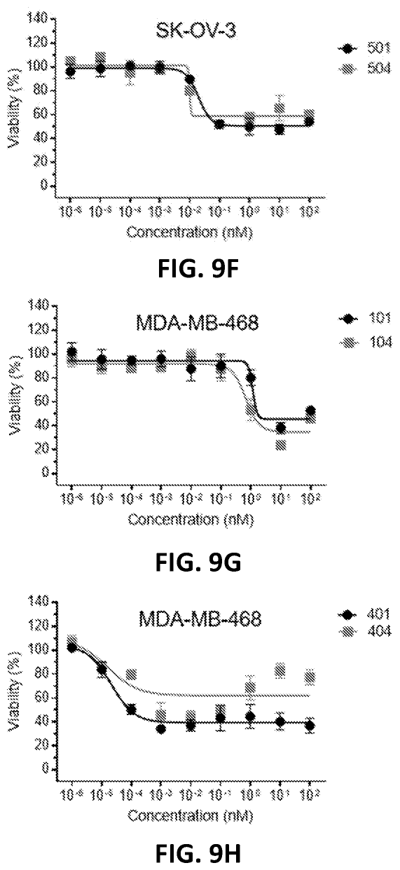

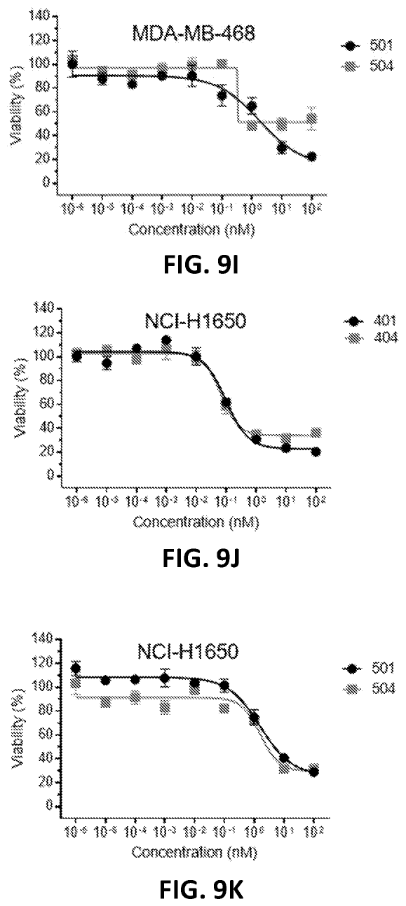

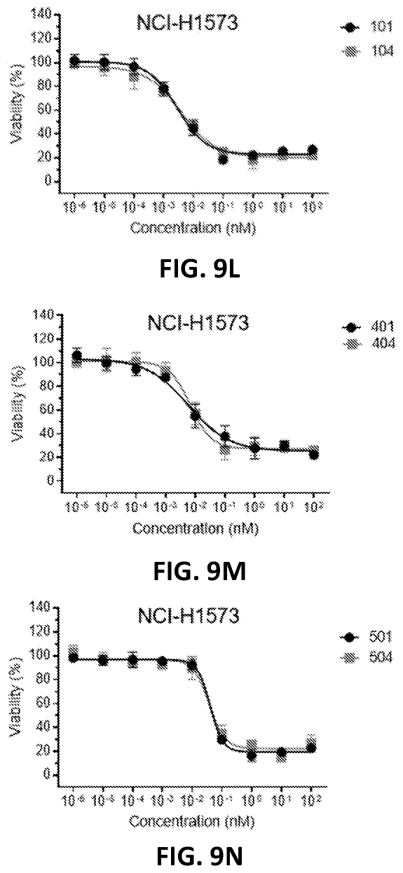

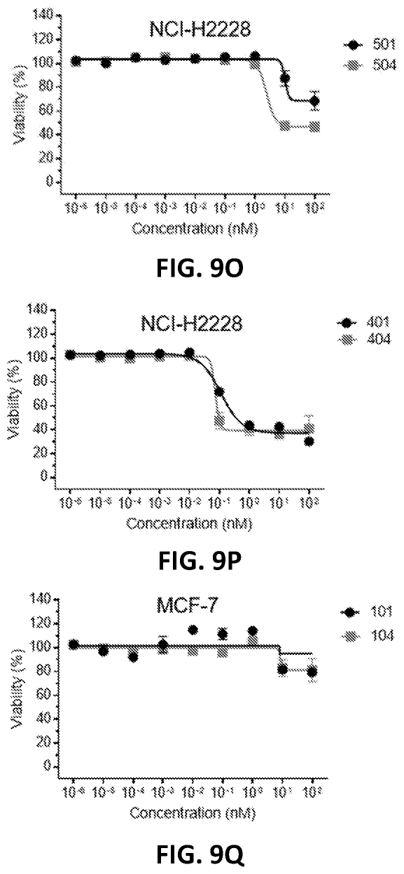

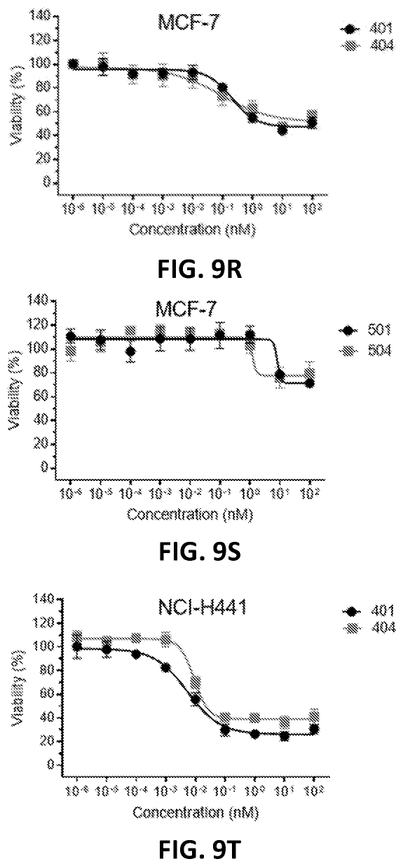

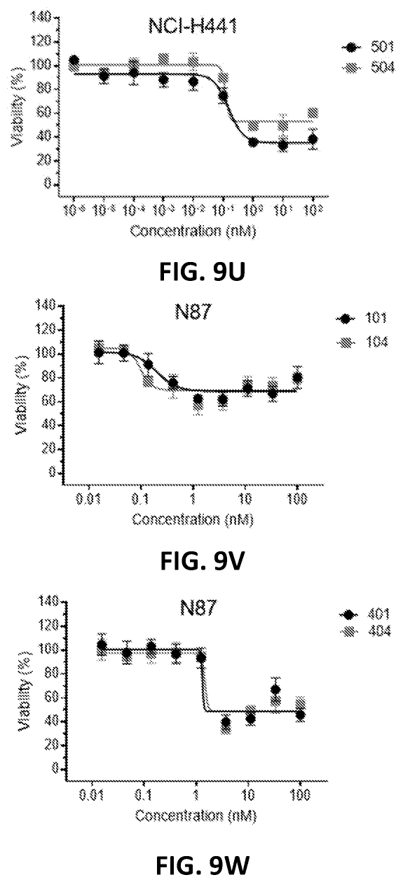

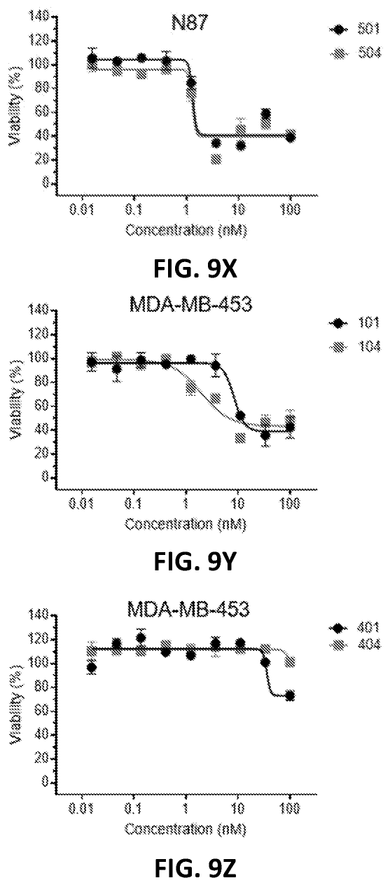

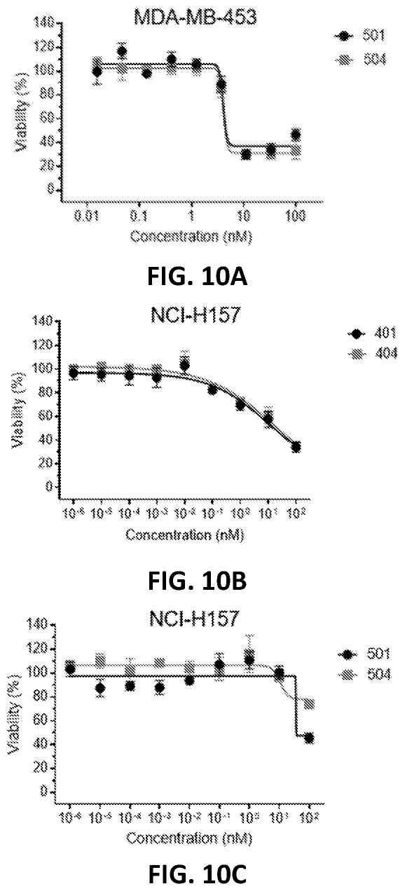

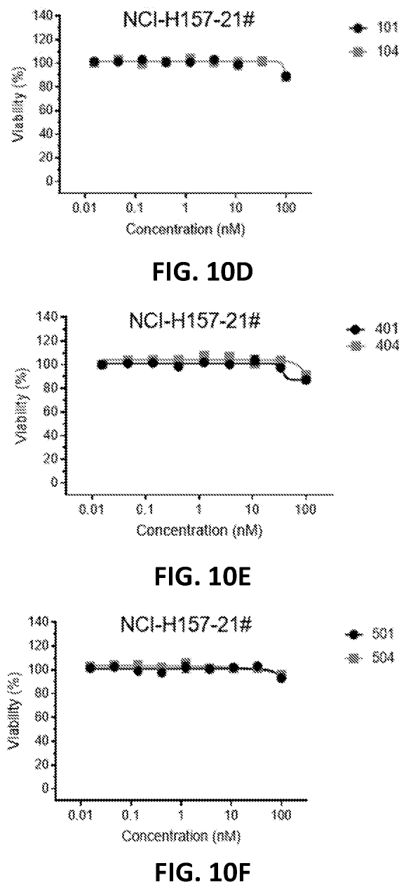

The data showed that the bispecific antibodies induced target-specific killing of tumor antigen-positive tumor cells (FIGS. 9A-9Z and 10A-10F). EC50 values (representing cell killing ability) calculated using GraphPad Prism are shown in the tables below.

TABLE 21
EC50 of tumor cell killing effect of bispecific antibodies

| | SK-BR-3 | SK-OV-3 | MDA-MB-468 | NCI-H1650 | NCI-H1573 | NCI-H2228 |
|---|---|---|---|---|---|---|
| Sample | EC50(nM) | | | | | |
| 101 | >100 | 1.005 | 1.164 | ND | 0.002872 | ND |
| 104 | >100 | 0.9866 | 0.6723 | ND | 0.003843 | ND |
| 401 | >100 | 0.01127 | 0.00002081 | 0.09859 | 0.005829 | 0.114 |
| 404 | >100 | 0.00995 | 0.00001575 | 0.06766 | 0.006942 | 0.07342 |
| 501 | >100 | 0.01979 | 1.789 | 1.586 | 0.04046 | 10.19 |
| 504 | >100 | 0.01001 | 0.3317 | 1.617 | 0.03778 | 2.433 |

TABLE 22
EC50 of tumor cell killing effect of bispecific antibodies

| | MCF-7 | NCI-H441 | N87 | MDA-MB-453 | NCI-H157-21# | NCI-H157 |
|---|---|---|---|---|---|---|
| Sample | EC50(nM) | | | | | |
| 101 | 8.007 | ND | 0.2003 | 8.351 | >100 | ND |
| 104 | 8.628 | ND | 0.09718 | 1.964 | >100 | ND |
| 401 | 0.2062 | 0.005384 | 1.312 | 35.81 | >100 | 10.02 |
| 404 | 0.08404 | 0.009077 | 1.468 | 142.1 | >100 | 17.73 |
| 501 | 8.29 | 0.1517 | 1.318 | 4.046 | >100 | 36.22 |
| 504 | 1.22 | 0.119 | 1.291 | 4.056 | >100 | 10.84 |

### Example 13: Thermal Stability of Bispecific Antibodies as Measured by DSF

The thermostability of the bispecific antibodies (401, 404, 501 and 504) was tested by Differential scanning fluorimetry (DSF). 19 μl of each protein sample (5 μM) was mixed with 1 μl of sypro orange (ThermoFisher, S6650) and added to 96-well plates (Applied Biosystems, N8010560) in triplicates. Reaction parameters: incubating at 4° C. for 2 min; incubating at 25° C. for 2 min; increasing the temperature from 25 to 95° C. at a rate of 0.05° C./min. The fluorescence signal intensity was measured by a real-time fluorescence quantitative PCR instrument (Applied Biosystems, QuantStudio 5). PBS was used as blank control. After the experiment, the Tm values were analyzed by the Protein Thermal Shift software.

The experiment results are shown in the table below. The Tm1 value of the bispecific antibodies is 62.2-63.0° C.; the Tm2 value is 72-74° C.

TABLE 23
Average Tm values of bispecific antibodies as measured by DSF

| Sample | Average Tm1 (° C.) | Average Tm2 (° C.) |
|---|---|---|
| 101 | 62.9 | 72.0 |
| 104 | 63.0 | 72.5 |
| 401 | 62.4 | 74.0 |
| 404 | 62.5 | 72.3 |
| 501 | 62.2 | 74.0 |
| 504 | 62.4 | 72.3 |

### Example 14: Using Salt-Gradient Affinity Capture Self-Interaction Nanoparticle Spectroscopy (SGAC-SINS) to Assess the Hydrophobic Interaction of Bispecific Antibodies

Reagents: gold nanoparticles (Ted Pella Inc, 15705-20); goat anti-human IgG Fe (Sigma, I2136); goat non-specific Ab (Jackson ImmunoResearch, 005-000-003); test antibodies (401, 404, 501 and 504); positive control antibody Ofatumumab; Negative control antibody Cixutumumab; PBS; KAc (pH 4.3); thiolated-PEG (Sigma, 729140).

Equipment: BioTek microplate reader (Biotek, SYNERGYH Lx Multi-made reader), 96-well plate (Corning, polystyrene UV transparent plate, 3635); PVDF syringe filter (0.22 m, Millex-GV, Millipore).

The experimental steps are as follows:

(1) Preparation of gold nanoparticle coating solution: Polyclonal goat anti-human IgG Fc antibodies (capture) and goat non-specific antibodies (non-capture) were buffer exchanged into 20 mM KAc (pH 4.3) and adjusted to a concentration of 0.4 mg/mL. A 4:1 volume ratio mix of capture:non-capture IgG solution was prepared to obtain immobilization of 80% capture antibody and 20% non-capture antibody.

(2) Preparation of the coated gold particles: A 9:1 volume ratio was used to mix gold nanoparticle solution (Ted Pella Inc, 15705-20) with the coating solution. After room temperature incubation for 1 h, thiolated PEG (Sigma Aldrich, 729140, final concentration 0.1 pM) was used to block empty sites on the nanoparticles to obtain coated gold particles.

(3) The coated gold particle solution was then passed through a 0.22 μm PVDF membrane (Millex-GV, 13 mm, Millipore). The coated gold particles were retained on top of the membrane and the flow-through solution was clear. PBS at 1/10 of the starting volume was used to elute the particles into the collection tube.

(4) 10 μL of the concentrated coated gold particles were added per well in 96-well plates, and 10 μL of test antibodies (1 mg/mL in PBS), control antibodies (1 mg/mL in PBS) and blank control PBS solution was added to each well. Each protein sample or PBS was added to 8 wells. The samples were incubated with the coated gold particles for 30 min.

(5) Then 90 μL of up to 1.22 M ammonium sulfate and 0.1 M sodium phosphate at pH 6.5 was added to the particles in the above 20 μL system, creating ammonium sulfate from 0.3 to 1 M in 0.1 M increments (0.3 M, 0.4 M, 0.5 M, 0.6 M, 0.7 M, 0.8 M, 0.9 M and 1 M). The particles were allowed to incubate for an additional 90 min.

(6) the 96-well plate was briefly centrifuged ((Eppendorf 5810R, at 1000 rpm for 1 min) to bring the solution menisci to the same level. Absorbance data was collected from 510 to 570 nm at increments of 2 nm, using a microplate reader (Biotek, SYNERGYH Lx Multi-made reader).

(7) Data processing: the highest absorbance wavelength at 510˜570 nm (from microplate reader) was identified. This wavelength was used as the center wavelength. A total of 20 data points (at different wavelengths) on the left and right sides of the center wavelength were recorded. Each data point was averaged with the data points directly before and after it to reduce error. Using the LINEST function in Microsoft Excel, a second-order polynomial was fit to the data. The coefficients were used to calculate the wavelength at which the slope is equal to zero, to determine whether or not this point is a maximum or minimum. In the case of a maximum, the calculated wavelength is recorded as the peak absorbance wavelength, unless it is greater than 560 nm. This peak absorbance wavelength was used as the y-axis and the ammonium sulfate concentration was used as the x-axis for graph analysis.

Evaluation criteria: (1) the antibody is poorly soluble if the antibody (1 mg/mL, PBS) shows peak absorbance at around 560 nm in low concentration (≤700 mM) ammonium sulfate solution; (2) the antibody is well soluble if the antibody shows no peak absorbance at around 560 nm in low concentration (≤700 mM) ammonium sulfate solution, and shows peak absorbance at around 560 nm only at a high concentration (>800 mM) of ammonium sulfate.

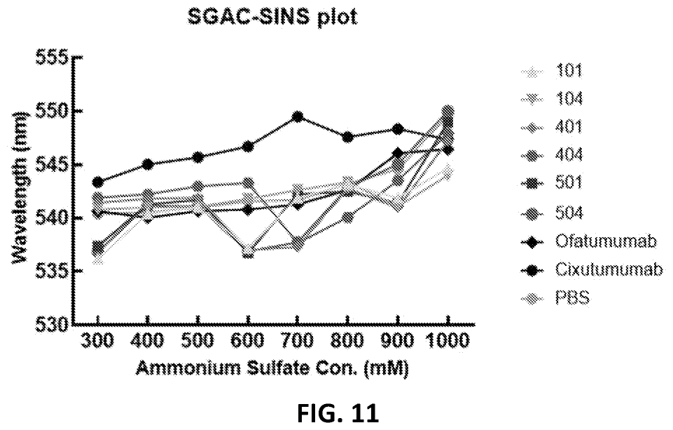

Results: The six antibodies, the positive control Ofatumumab, and the blank control PBS did not show peak absorbance at around 560 nm in low concentrations of ammonium sulfate, indicating that these molecules were well soluble. The negative control molecule, Cixutumumab, showed peak absorbance at around 560 nm in low concentrations of ammonium sulfate, indicating poor solubility. The results are shown in FIG. 11 and the table below.

TABLE 24
SGAC-SINS detection results of bispecific antibodies (peak absorbance nm)

| Ammonium | | | | | | | | | |
|---|---|---|---|---|---|---|---|---|---|
| Sulfate (mM) | 101 | 104 | 401 | 404 | 501 | 504 | Ofatumumab | Cixutumumab | PBS |
| 300 | 536.2 | 540.8 | 541.4 | 541.9 | 537.4 | 536.8 | 540.6 | 543.4 | 540.3 |
| 400 | 540.4 | 541 | 541.8 | 542.2 | 541.3 | 541.2 | 540 | 545 | 540.5 |
| 500 | 541 | 541 | 541.9 | 543 | 541.7 | 541.1 | 540.6 | 545.7 | 540.9 |
| 600 | 537.2 | 541.8 | 537 | 543.3 | 536.7 | 536.9 | 540.8 | 546.7 | 541.6 |
| 700 | 541.9 | 542.6 | 537.3 | 537.6 | 542.2 | 537.7 | 541.3 | 549.5 | 541.7 |
| 800 | 543.2 | 543.4 | 542.8 | 540.1 | 542.5 | 542.9 | 542.6 | 547.6 | 543.2 |
| 900 | 541.9 | 541.1 | 541 | 543.5 | 541.4 | 545 | 546.1 | 548.3 | 544.6 |
| 1000 | 544.7 | 544 | 547.2 | 547.9 | 549 | 550 | 546.5 | 547.4 | 549.6 |

### Example 15: Detection of Self-Interaction of Bispecific Antibodies by Bio-Layer Interferometry (BLI) Assay

Bio-Layer Interferometry (BLI) assays were performed on OCTECT RED96e (ForteBio) at 30° C. with PBS running buffer (10 mmol/L Na2HPO4; 1.75 mmol/L KH2PO4; 137 mmol/L NaCl; 2.65 mmol/L KCl; pH 7.2-7.4).

Bispecific antibodies and control antibodies (Adalimumab, Ofatumumab, etc.) were first captured on the surface of an AHQ sensor (Anti-hIgG Fc, sartorius, 18-5001) chip with immobilized anti-human Fc antibodies. The bispecific antibodies were diluted to 1 μM, and the proteins were coupled to the surface of the AHQ sensor chip in PBS, and the signal value was ˜0.8 nm. Subsequently, the sites on the AHQ sensor that were not bound to the test antibodies were fully blocked with human IgG antibody, and the self-binding signal of the bispecific antibodies or the control antibodies was analyzed. The experimental parameters are as follows: Baseline1: 60s, Loading: 180s, Loading response: 0.8 nm, Baseline2: 180s, Association: 240s, High sensitivity kinetics: 2 Hz. Binding curves were obtained using the Date Analysis HT 12 software (sartorius, 50-5029). If the self-binding signal of the test antibody is higher than that of the control antibody by more than 0.1 nm, the test antibody is considered to have self-interaction; otherwise, it is considered to not have self-interaction. The experimental results are shown in the table below.

TABLE 25
BLI detection of self-interacting signals of bispecific antibodies

| Sample | BLI-Response | |
|---|---|---|
| 101 | 0.21 | nm |
| 104 | 0.22 | nm |
| 401 | 0.043 | nm |
| 404 | 0.062 | nm |
| 501 | 0.056 | nm |
| 504 | 0.07 | nm |
| Ofatumumab | 0.09 | nm |

The positive control Ofatumumab showed a self-interaction signal of 0.09 nm. The self-interaction signals of samples 401, 501, 404 and 504 were in the range of 0.04-0.07 nm, which are less than 0.19 nm (0.09 nm+0.1 nm). Thus, the test antibodies 401, 404, 501 and 504 can be judged as having weak self-interaction, suggesting that the molecules are soluble. Although the self-interaction signals of samples 101 and 104 are larger than 0.19 nm, they are only 0.21 and 0.22 nm, and the self-interaction signals are considered to be not strong.

### Example 16: Detection of Specificity of Bispecific Antibodies by ELISA

The experimental steps are as follows

(1) Antigen coating: cardiolipin (Sigma, cat. C0563), keyhole limpet haemocyanin (KLH, Sigma, H8283), LPS (Sigma, L6529), ssDNA (Sigma, D8899), dsDNA (Sigma, D4522) and Insulin (abs42019847) were plated at 50 μg/mL, 5 μg/mL, 10 μg/mL, 1 μg/mL, 1 μg/mL, g/mL, and 50 μL per well, and incubated at 4° C. overnight;

(2) The next morning, the plate was washed three times with 0.05% PBST (Biotek, Biotek, 4052S Microplate washer); blocked in PBS solution containing 3% skim milk for 1 h at room temperature, and washed three times with 0.05% PBST;

(3) 50 μl of 100 nM antibodies or blank control PBS (3 replicate wells for each sample) were added to each well and incubated for 1 h at room temperature; the plate was washed 3 times with 0.05% PBST;

(4) 50 μl anti-human IgG-HRP conjugate (Sigma, AP113P, 1:8000 dilution) was added to each well; the plate was incubated for 1 h at room temperature and washed 6 times with 0.05% PBST;

(5) 50 μl of one-component TMB chromogenic solution (Biopanda, TMB-S-001) was added to each well and incubated at room temperature for 10-15 minutes for color development;

(6) 50 μl of 1M HCl was added to each well to stop the color development, and the absorbance at 450 nm was measured using a microplate reader (Biotek, SYNERGYH Lx Multi-made reader);

Data analysis: If the ratio between the absorbance of the antibody and the absorbance of the blank control PBS is ≥3, there is too much non-specific binding.

The experimental results are shown in the table below. From the results, it can be seen that the none of the tested antibodies showed too much non-specific binding.

TABLE 26
Absorbance values of bispecific antibodies
combined with various substances

| Sample | Cardiolipin | KLH | LPS | ssDNA | dsDNA | Insulin |
|---|---|---|---|---|---|---|
| 101 | 1.96 | 1.05 | 1.32 | 1.80 | 1.06 | 2.02 |
| 104 | 1.79 | 1.08 | 1.33 | 1.51 | 1.15 | 2.88 |
| 401 | 1.02 | 0.96 | 0.86 | 1.64 | 1.12 | 1.01 |
| 404 | 1.22 | 1.06 | 0.94 | 2.89 | 1.56 | 1.08 |
| 501 | 1.74 | 1.45 | 1.03 | 2.68 | 1.50 | 1.52 |
| 504 | 1.86 | 1.92 | 1.47 | 1.66 | 1.93 | 1.51 |
| Ofatumumab | 1.06 | 1.63 | 1.05 | 0.99 | 1.02 | 1.02 |

### Example 17: Anti-Tumor Effects of CEA-CD3 and HER2-CD3 Bispecific Antibodies in LS174T Human Colon Cancer Co-Implanted with Human PBMCs

Female NCG (NOD/ShiLtJGpt-Prkdc^{em26Cd52}Il2rg^{em26Cd22}/Gpt, GemPharmatech, Strain NO. T001475) mice (n=5) were injected subcutaneously with 1×10⁶ LS174T cells premixed with human PBMC (E:T ratio 5:1, total volume 200 μl). In order to evaluate the effect of bispecific antibody treatment, mice were intravenous injected with 1.0 mg/kg bispecific antibodies once a week, starting from 1 h after subcutaneous co-transplantation of tumor cells/PBMCs. In the vehicle control group (vehicle), PBS buffer was administered instead of antibody administration. A total of 3 doses were administered. Tumor volumes were measured weekly with digital calipers, and body weights were recorded. The relative change of body weight RCB (%) (Relative Change of Body weight) was calculated based on the below formula: RCB (%)=[1−(Bi/B0)×100%](Bi: mean body weight at day i, B0: average body weight at day 0). At the same time, the tumor volume (long diameter×short diameter 2/2) was determined and the growth inhibition rate TGI_{TV} (%) (tumor growth inhibition %) was calculated based on the below formula: TGI_{TV} (%)=[1−(Ti−T0)/(Vi−V0)]×100% (Ti: the mean tumor volume of the treatment group on day i, T0: the first measurable tumor volume of the treatment group; Vi: the mean tumor volume of the vehicle control group on day i, V0: The mean tumor volume that can be measured for the first time in the vehicle control group).

The experimental results are shown as mean±standard error (Mean±SEM), and graphs were drawn using GraphPad Prism 9.0 software, and two-way ANOVA was performed. P<0.05 indicated that the difference was statistically significant.

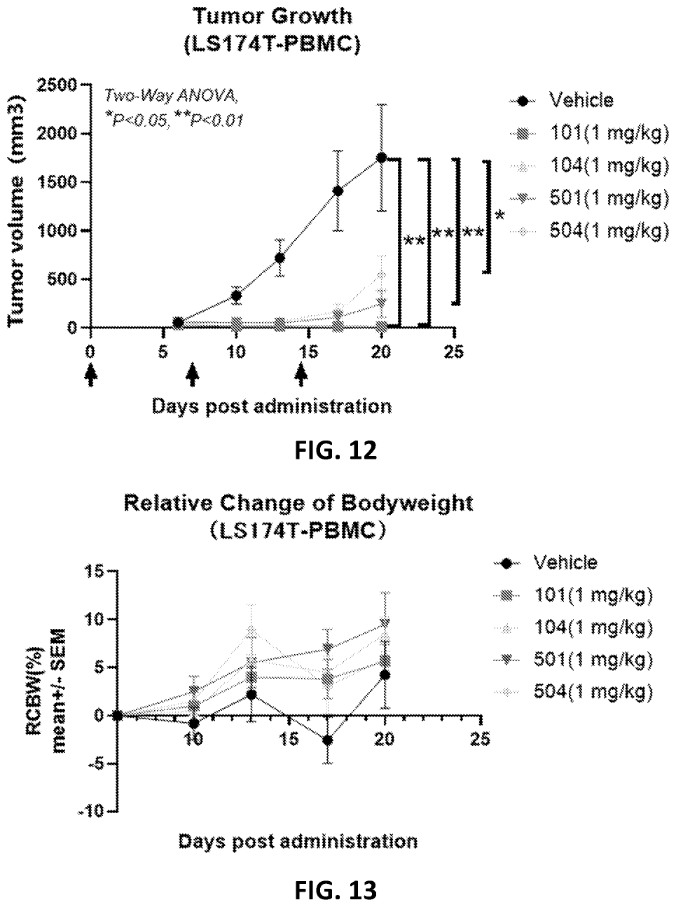

On the 20th day of administration, compared with the PBS control group, CEA-CD3 and HER2-CD3 bispecific antibodies showed significant inhibitory effect on tumor volume, with statistical difference (P<0.05 or P<0.01). At the 1 mg/kg dose, the tumor growth inhibition rates (TGI_{TV}) of 101, 104, 501 and 504 were 100.96%, 102.75%, 88.98% and 71.67%, respectively, showing strong anti-tumor inhibitory effects. See FIG. 12 (arrows represent dosing) and the table below.

TABLE 27
Effects of CEA-CD3 and HER2-CD3 bispecific antibodies
on tumor volume in PBMC/LS174T inoculated NCG mice

| | Tumor Volume (mm³) | | | | | |
|---|---|---|---|---|---|---|
| Sample | Day 6 | Day 10 | Day 13 | Day 17 | Day 20 | TGI_{TV}(%) |
| Vehicle | 58.15 ± 15.73 | 334.34 ± 90.27 | 721.89 ± 184.74 | 1410.49 ± 411.52 | 1753.31 ± 548.25 | — |
| 101 (1 | 32.08 ± 13.14 | 18.74 ± 11.48 | 18.28 ± 11.71 | 17.78 ± 10.93 | 15.76 ± 9.65 | 100.96% |
| mg/kg) | | | | | | |
| 104 (1 | 56.21 ± 10.46 | 19.49 ± 10.85 | 18.19 ± 11.86 | 11.17 ± 7.23 | 9.54 ± 6.12 | 102.75% |
| mg/kg) | | | | | | |
| 501 (1 | 61.8 ± 16.3 | 57.12 ± 14.66 | 52.91 ± 16.15 | 111.78 ± 64.72 | 248.57 ± 140.27 | 88.98% |
| mg/kg) | | | | | | |
| 504 (1 | 73.31 ± 14.05 | 51.26 ± 12.49 | 68.43 ± 19.64 | 167.35 ± 79.83 | 553.48 ± 188.24 | 71.67% |
| mg/kg) | | | | | | |

During the administration period, the NCG mice showed normal physical activity and food intake, and the relative body weight change rate was within ±10%. On day 20, the weight of the mice increased by 0.90-1.94 g, and the weight of the mice in the administration group increased significantly. The above results showed that the mice tolerated the administration frequency and dose, and the drug safety was good. See FIG. 13 and the table below.

TABLE 28
Effects of CEA-CD3 and HER2-CD3 bispecific antibodies
on the body weight of PBMC/LS174T inoculated NCG mice

| | Average Weight (g) | | | | | Weight change |
|---|---|---|---|---|---|---|
| Sample | Day 6 | Day 10 | Day 13 | Day 17 | Day 20 | in 20 days (g) |
| Vehicle | 21.73 ± 0.35 | 21.54 ± 0.33 | 22.17 ± 0.41 | 21.16 ± 0.51 | 22.63 ± 0.72 | +0.90 |
| 101 (1 mg/kg) | 21.71 ± 0.48 | 21.9 ± 0.22 | 22.56 ± 0.39 | 22.51 ± 0.36 | 22.92 ± 0.46 | +1.21 |
| 104 (1 mg/kg) | 22.16 ± 0.35 | 22.26 ± 0.41 | 23.43 ± 0.6 | 23.14 ± 0.36 | 24.03 ± 0.46 | +1.87 |
| 501 (1 mg/kg) | 20.68 ± 0.44 | 21.18 ± 0.34 | 21.8 ± 0.5 | 22.09 ± 0.48 | 22.62 ± 0.64 | +1.94 |
| 504 (1 mg/kg) | 20.87 ± 0.63 | 21.15 ± 0.49 | 22.68 ± 0.55 | 21.43 ± 0.36 | 22.05 ± 0.34 | +1.18 |

### Example 18: Antitumor Effect of EGFR-CD3 Bispecific Antibody in HT-29 Human Colon Cancer Cells Co-Planted with Human PBMC

Female NCG (NOD/ShiLtJGpt-Prkdc^{em26Cd52}Il2rg^{em26Cd22}/Gpt, GemPharmatech, Strain NO. T001475) mice (n=5) were injected subcutaneously with 1×10⁶ HT-29 cells premixed with human PBMC (E:T ratio 1:1, total volume 200 μl). In order to evaluate the effect of bispecific antibody treatment, mice received intravenous injection of 1.0 mg/kg bispecific antibody once a week, starting 1 h after subcutaneous co-transplantation of tumor cells/PBMCs. In a vehicle control group (vehicle), PBS buffer was administered instead of antibody administration. A total of 3 doses were administered. Tumor volumes were measured weekly with digital calipers, and body weights were recorded. The relative body weight change rate RCB (%) (Relative Change of Body weight) was calculated based on the below formula: RCB (%)=[1−(Bi/B0)×100%](Bi: mean weight on day i, B0: mean body weight at day 0). At the same time, the tumor volume (long diameter×short diameter 2/2) was determined and growth inhibition rate TGI_{TV}(%)(tumor growth inhibition %) was calculated based on the below formula: TGI_{TV}(%)=[1−(Ti−T0)/(Vi−V0)]×100% (Ti: the mean tumor volume of the treatment group on day i, T0: the first measurable tumor volume of the treatment group; Vi: the mean tumor volume of the vehicle control group on day i, V0: The mean tumor volume that can be measured for the first time in the vehicle control group).

The experimental results are shown as mean±standard error (Mean±SEM), and graphs were drawn using GraphPad Prism 9.0 software, and two-way ANOVA was performed. P<0.05 indicated that the difference was statistically significant.

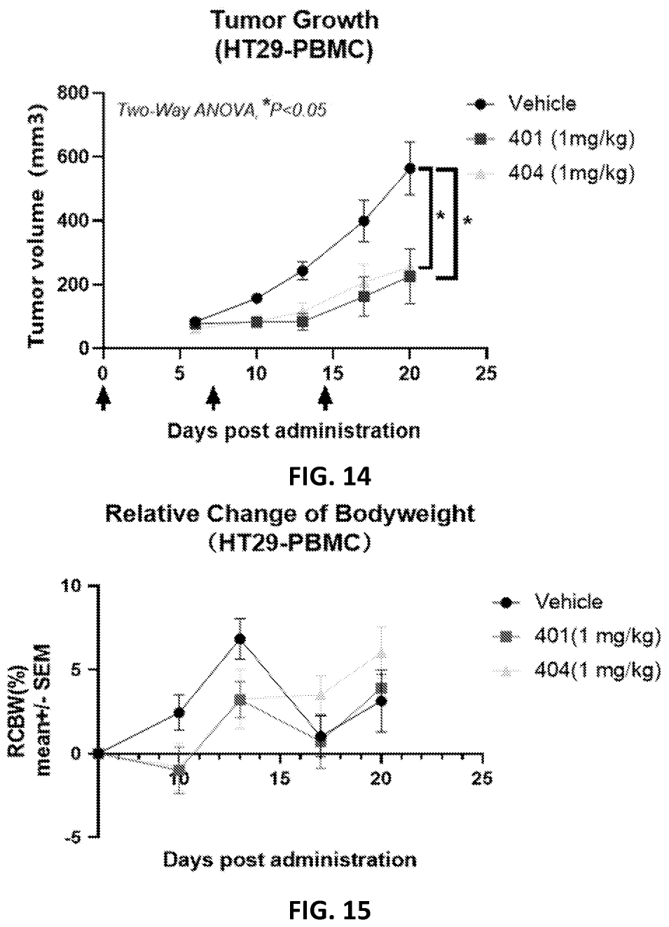

On the 20th day after administration, compared with the PBS control group, the EGFR-CD3 bispecific antibody had a significant inhibitory effect on the tumor volume, with a statistical difference (P<0.05). At the dose of 1 mg/kg, the tumor growth inhibition rates (TGI_{TV}) of 401 and 404 were 69.09% and 59.83%, respectively, indicating strong anti-tumor inhibitory effects. See FIG. 14 (arrows represent dosing) and the table below.

TABLE 29
Effect of EGFR-CD3 bispecific antibody on tumor volume in PBMC/HT-29 inoculated NCG mice

| | Tumor Volume (mm³) | | | | | |
|---|---|---|---|---|---|---|
| Sample | Day 6 | Day 10 | TGI_{TV}(% | Day 17 | Day 20 | TGI_{TV}(% |
| Vehicle | 84.22 ± 11.79 | 157.48 ± 13.64 | 243.41 ± 28.01 | 399.69 ± 64.65 | 564.36 ± 82.55 | |
| 401 (1 | 77.98 ± 10.63 | 83.53 ± 13.52 | 84.54 ± 26.15 | 163.00 ± 61.26 | 226.46 ± 86.35 | 69.09% |
| mg/kg) | | | | | | |
| 404 (1 | 63.39 ± 11.69 | 85.21 ± 20.23 | 115.69 ± 28.34 | 208.62 ± 55.69 | 256.26 ± 58.6 | 59.83% |
| mg/kg) | | | | | | |

During the administration period, the NCG mice showed normal physical activity and food intake, and the relative body weight change rate was within ±10%. On the 20th day after administration, the weight of mice increased by 0.66-1.10 g, and the weight of mice in the administration group increased significantly. The above results showed that the mice tolerated the administration frequency and dose, and the drug safety was good. See FIG. 15 and the table below.

TABLE 30
Effects of EGFR-CD3 bispecific antibody on body weight of PBMC/HT-29 inoculated NCG mice

| | Average Weight (g) | | | | | Weight change |
|---|---|---|---|---|---|---|
| Sample | Day 6 | Day 10 | Day 13 | Day 17 | Day 20 | in 20 days (g) |
| Vehicle | 21.56 ± 0.37 | 22.09 ± 0.41 | 23.02 ± 0.17 | 21.77 ± 0.28 | 22.22 ± 0.32 | +0.66 |
| 401 (1 mg/kg) | 22.63 ± 0.37 | 22.39 ± 0.28 | 23.35 ± 0.39 | 22.78 ± 0.39 | 23.5 ± 0.31 | +0.87 |
| 404 (1 mg/kg) | 21.62 ± 0.57 | 21.75 ± 0.66 | 22.48 ± 0.68 | 22.38 ± 0.69 | 22.72 ± 0.93 | +1.10 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antigen-binding protein, comprising
(a) a Fc;
(b) a Fab fragment (Fab) that specifically binds to a T cell antigen; and
(c) a single-domain antibody variable domain (VHH) that specifically binds to a tumor-associated antigen,
wherein the Fab and the VHH are linked to the Fc.

2. The antigen-binding protein of claim 1, wherein the Fab comprises or consists of a light chain variable domain (VL), a light chain constant domain (CL), a heavy chain variable domain (VH), and a heavy chain first constant domain (CH1).

3. The antigen-binding protein of claim 2, wherein the Fab can activate T cells upon binding to the T cell antigen.

4. The antigen-binding protein of any one of claims 1-3, wherein the T cell antigen is cluster of differentiation 3 (CD3).

5. The antigen-binding protein of any one of claims 1-4, wherein the tumor-associated antigen is cluster of differentiate 20 (CD20), prostate-specific antigen (PSA), prostate stem cell antigen (PSCA), programmed death-ligand 1 (PD-L1), human epidermal growth factor receptor 2 (Her2), human epidermal growth factor receptor 3 (Her3), human epidermal growth factor receptor (Her1), β-Catenin, cluster of differentiate 19 (CD19), epidermal growth factor receptor (EGFR), tyrosine-protein kinase Met (c-Met), epithelial cell adhesion molecule (EPCAM), prostate-specific membrane antigen (PSMA), cluster of differentiate 40 (CD40), Mucin 1, Cell Surface Associated (MUC1), insulin-like growth factor 1 receptor (IGF1R), or carcinoembryonic antigen cell adhesion molecule 5 (CEACAM5).

6. The antigen-binding protein of any one of claims 1-5, wherein the Fc is human IgG4 Fc.

7. The antigen-binding protein of any one of claims 2-6, wherein the CH1 domain of the Fab is linked to a CH2 domain in the Fc, optionally via a hinge region.

8. The antigen-binding protein of claim 7, wherein the hinge region is a human IgG4 hinge region optionally with S228P mutation according to EU numbering.

9. The antigen-binding protein of any one of claims 2-6, wherein the Fab is linked to the C-terminus of a CH3 domain in the Fc.

10. The antigen-binding protein of claim 9, wherein the Fab is linked to the CH3 domain via a linker peptide.

11. The antigen-binding protein of any one of claims 1-10, wherein the VHH is linked to a CH2 domain in the Fc, optionally via a hinge region.

12. The antigen-binding protein of claim 11, wherein the hinge region is a human IgG4 hinge region optionally with S228P mutation according to EU numbering.

13. The antigen-binding protein of any one of claims 1-10, wherein the VHH is linked to the C-terminus of a CH3 domain in the Fc.

14. The antigen-binding protein of claim 13, wherein the VHH is linked to the CH3 domain via a linker peptide.

15. The antigen-binding protein of any one of claims 1-14, wherein the Fc comprises a first polypeptide and a second polypeptide, wherein each polypeptide comprises one or more knobs-into-holes mutations.

16. A protein complex, comprising:
(a) a first polypeptide comprising from N-terminus to C-terminus: a VH, a CH1 domain, optionally a first hinge region, a first CH2 domain, and a first CH3 domain;
(b) a second polypeptide comprising from N-terminus to C-terminus: a VHH, optionally a second hinge region, a second CH2 domain, and a second CH3 domain;
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

17. The protein complex of claim 16, wherein the T cell antigen is CD3, and the tumor-associated antigen is CEACAM5.

18. The protein complex of claim 16 or 17, wherein the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 11; and the third polypeptide comprises a sequence that is at least 80%, 90%, 95%, or 100% identical to SEQ ID NO: 23.

19. A protein complex, comprising:
(a) a first polypeptide comprising from N-terminus to C-terminus: a VH, a CH1 domain, optionally a first hinge region, a first CH2 domain, a first CH3 domain, optionally a linker peptide, and a single-domain antibody variable domain (VHH);
(b) a second polypeptide comprising from N-terminus to C-terminus: optionally a second hinge region, a second CH2 domain, and a second CH3 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

20. The protein complex of claim 19, wherein the T cell antigen is CD3, and the tumor-associated antigen is CEACAM5.

21. The protein complex of claim 19 or 20, wherein the first polypeptide comprises a sequence that is at least 80%, 90%, 95%, or 100% identical to SEQ ID NO: 6; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 23.

22. A protein complex, comprising:
(a) a first polypeptide comprising from N-terminus to C-terminus: a VH, a CH1 domain, optionally a first hinge region, a first CH2 domain, and a first CH3 domain;
(b) a second polypeptide comprising from N-terminus to C-terminus: optionally a second hinge region, a second CH2 domain, a second CH3 domain, optionally a linker peptide, and a single-domain antibody variable domain (VHH); and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

23. The protein complex of claim 22, wherein the T cell antigen is CD3, and the tumor-associated antigen is CEACAM5.

24. The protein complex of claim 22 or 23, wherein the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 12; and the third polypeptide comprises a sequence that is at least 80%, 90%, 95%, or 100% identical to SEQ ID NO: 23.

25. A protein complex, comprising:
(a) a first polypeptide comprising from N-terminus to C-terminus: a single-domain antibody variable domain (VHH), optionally a first hinge region, a first CH2 domain, and a first CH3 domain;
(b) a second polypeptide comprising from N-terminus to C-terminus: a VH, a CH1 domain, optionally a second hinge region, a second CH2 domain, and a second CH3 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

26. The protein complex of claim 25, wherein the T cell antigen is CD3, and the tumor-associated antigen is CEACAM5.

27. The protein complex of claim 25 or 26, wherein the first polypeptide comprises a sequence that is at least 80%, 90%, 95%, or 100% identical to SEQ ID NO: 4; the second polypeptide comprises a sequence that is at least 80%, 90%, 95%, or 100% identical to SEQ ID NO: 9;
and the third polypeptide comprises a sequence that is at least 80%, 90%, 95%, or 100% identical to SEQ ID NO: 23.

28. A protein complex, comprising:
(a) a first polypeptide comprising from N-terminus to C-terminus: optionally a first hinge region, a first CH2 domain, a first CH3 domain, optionally a linker peptide, and a single-domain antibody variable domain (VHH);
(b) a second polypeptide comprising from N-terminus to C-terminus: a VH, a CH1 domain, optionally a second hinge region, a second CH2 domain, and a second CH3 domain;
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

29. The protein complex of claim 28, wherein the T cell antigen is CD3, and the tumor-associated antigen is CEACAM5.

30. The protein complex of claim 28 or 29, wherein the first polypeptide comprises a sequence that is at least 80%, 90%, 95%, or 100% identical to SEQ ID NO: 5; the second polypeptide comprises a sequence that is at least 80%, 90%, 95%, or 100% identical to SEQ ID NO: 9;
and the third polypeptide comprises a sequence that is at least 80%, 90%, 95%, or 100% identical to SEQ ID NO: 23.

31. A protein complex, comprising:
(a) a first polypeptide comprising from N-terminus to C-terminus: optionally a first hinge region, a first CH2 domain, and a first CH3 domain;
(b) a second polypeptide comprising from N-terminus to C-terminus: a VH, a CH1 domain, optionally a second hinge region, a second CH2 domain, a second CH3 domain, optionally a linker peptide, and a single-domain antibody variable domain (VHH); and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

32. The protein complex of claim 31, wherein the T cell antigen is CD3, and the tumor-associated antigen is CEACAM5.

33. The protein complex of claim 31 or 32, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 13; and the third polypeptide comprises a sequence that is at least 80%, 90%, 95%, or 100% identical to SEQ ID NO: 23.

34. A protein complex, comprising:
(a) a first polypeptide comprising from N-terminus to C-terminus: a single-domain antibody variable domain (VHH), optionally a first hinge region, a first CH2 domain, a first CH3 domain, optionally a linker peptide, a VH, and a CH1 domain;
(b) a second polypeptide comprising from N-terminus to C-terminus: optionally a second hinge region, a second CH2 domain, and a second CH3 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

35. The protein complex of claim 34, wherein the T cell antigen is CD3, and the tumor-associated antigen is CEACAM5.

36. The protein complex of claim 34 or 35, wherein the first polypeptide comprises a sequence that is at least 80%, 90%, 95%, or 100% identical to SEQ ID NO: 7; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 23.

37. A protein complex, comprising:
(a) a first polypeptide comprising from N-terminus to C-terminus: optionally a first hinge region, a first CH2 domain, a first CH3 domain, optionally a linker peptide, a VH, and a CH1 domain;
(b) a second polypeptide comprising from N-terminus to C-terminus: a single-domain antibody variable domain (VHH), optionally a second hinge region, a second CH2 domain, and a second CH3 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

38. The protein complex of claim 37, wherein the T cell antigen is CD3, and the tumor-associated antigen is CEACAM5.

39. The protein complex of claim 37 or 38, wherein the first polypeptide comprises a sequence that is at least 80%, 90%, 95%, or 100% identical to SEQ ID NO: 3; the second polypeptide comprises a sequence that is at least 80%, 90%, 95%, or 100% identical to SEQ ID NO: 11; and the third polypeptide comprises a sequence that is at least 80%, 90%, 95%, or 100% identical to SEQ ID NO: 23.

40. A protein complex, comprising:
(a) a first polypeptide comprising from N-terminus to C-terminus: optionally a first hinge region, a first CH2 domain, a first CH3 domain, optionally a first linker peptide, a VH, and a CH1 domain;
(b) a second polypeptide comprising from N-terminus to C-terminus: optionally a second hinge region, a second CH2 domain, a second CH3 domain, optionally a second linker peptide, and a single-domain antibody variable domain (VHH); and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

41. The protein complex of claim 40, wherein the T cell antigen is CD3, and the tumor-associated antigen is CEACAM5.

42. The protein complex of claim 40 or 41, wherein the first polypeptide comprises a sequence that is at least 80%, 90%, 95%, or 100% identical to SEQ ID NO: 3; the second polypeptide comprises a sequence that is at least 80%, 90%, 95%, or 100% identical to SEQ ID NO: 12; and the third polypeptide comprises a sequence that is at least 80%, 90%, 95%, or 100% identical to SEQ ID NO: 23.

43. A protein complex, comprising:
(a) a first polypeptide comprising from N-terminus to C-terminus: a single-domain antibody variable domain (VHH), optionally a first hinge region, a first CH2 domain, and a first CH3 domain;
(b) a second polypeptide comprising from N-terminus to C-terminus: optionally a second hinge region, a second CH2 domain, a second CH3 domain, optionally a linker peptide, a VH, and a CH1 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

44. The protein complex of claim 43, wherein the T cell antigen is CD3, and the tumor-associated antigen is CEACAM5.

45. The protein complex of claim 43 or 44, wherein the first polypeptide comprises a sequence that is at least 80%, 90%, 95%, or 100% identical to SEQ ID NO: 4; the second polypeptide comprises a sequence that is at least 80%, 90%, 95%, or 100% identical to SEQ ID NO: 10; and the third polypeptide comprises a sequence that is at least 80%, 90%, 95%, or 100% identical to SEQ ID NO: 23.

46. A protein complex, comprising:
(a) a first polypeptide comprising from N-terminus to C-terminus: optionally a first hinge region, a first CH2 domain, and a first CH3 domain;
(b) a second polypeptide comprising from N-terminus to C-terminus: a single-domain antibody variable domain (VHH), optionally a second hinge region, a second CH2 domain, a second CH3 domain, optionally a linker peptide, a VH, and a CH1 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

47. The protein complex of claim 46, wherein the T cell antigen is CD3, and the tumor-associated antigen is CEACAM5.

48. The protein complex of claim 46 or 47, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 14; and the third polypeptide comprises a sequence that is at least 80%, 90%, 95%, or 100% identical to SEQ ID NO: 23.

49. A protein complex, comprising:
(a) a first polypeptide comprising from N-terminus to C-terminus: optionally a first hinge region, a first CH2 domain, a first CH3 domain, optionally a first linker peptide, and a single-domain antibody variable domain (VHH);
(b) a second polypeptide comprising from N-terminus to C-terminus: optionally a second hinge region, a second CH2 domain, a second CH3 domain, optionally a second linker peptide, a VH, and a CH1 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

50. The protein complex of claim 49, wherein the T cell antigen is CD3, and the tumor-associated antigen is CEACAM5.

51. The protein complex of claim 49 or 50, wherein the first polypeptide comprises a sequence that is at least 80%, 90%, 95%, or 100% identical to SEQ ID NO: 5; the second polypeptide comprises a sequence that is at least 80%, 90%, 95%, or 100% identical to SEQ ID NO: 10; and the third polypeptide comprises a sequence that is at least 80%, 90%, 95%, or 100% identical to SEQ ID NO: 23.

52. A protein complex, comprising:
(a) a first polypeptide comprising from N-terminus to C-terminus: a VH, a CH1 domain, optionally a first hinge region, a first CH2 domain, and a first CH3 domain;
(b) a second polypeptide comprising from N-terminus to C-terminus: optionally a second hinge region, a second CH2 domain, and a second CH3 domain; and
(c) a third polypeptide comprising from N-terminus to C-terminus: a VL, a CL domain, optionally a third linker peptide, a single-domain antibody variable domain (VHH),
wherein the VH and the VL associate with each other, forming an antigen binding site of a Fab that specifically binds to a T cell antigen, wherein the VHH specifically binds to a tumor-associated antigen.

53. The protein complex of claim 52, wherein the T cell antigen is CD3, and the tumor-associated antigen is CEACAM5.

54. The protein complex of claim 52 or 53, wherein the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 39.

55. A protein complex, comprising:
(a) a first polypeptide comprising from N-terminus to C-terminus: optionally a first hinge region, a first CH2 domain, and a first CH3 domain;
(b) a second polypeptide comprising from N-terminus to C-terminus: a VH, a CH1 domain, optionally a second hinge region, a second CH2 domain, and a second CH3 domain; and
(c) a third polypeptide comprising from N-terminus to C-terminus: a VL, a CL domain, optionally a third linker peptide, and a single-domain antibody variable domain (VHH),
wherein the VH and the VL associate with each other, forming an antigen binding site of a Fab that specifically binds to a T cell antigen, wherein the VHH specifically binds to a tumor-associated antigen.

56. The protein complex of claim 55, wherein the T cell antigen is CD3, and the tumor-associated antigen is CEACAM5.

57. The protein complex of claim 55 or 56, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 9; and the third polypeptide comprises a sequence that is at least 80%, 90%, 95%, or 100% identical to SEQ ID NO: 39.

58. A protein complex, comprising:
(a) a first polypeptide comprising from N-terminus to C-terminus: a VH, a CH1 domain, optionally a first hinge region, a first CH2 domain, and a first CH3 domain;
(b) a second polypeptide comprising from N-terminus to C-terminus: optionally a second hinge region, a second CH2 domain, and a second CH3 domain; and
(c) a third polypeptide comprising from N-terminus to C-terminus: a single-domain antibody variable domain (VHH), optionally a third linker peptide, a VL, and a CL domain,
wherein the VH and the VL associate with each other, forming an antigen binding site of a Fab that specifically binds to a T cell antigen, wherein the VHH specifically binds to a tumor-associated antigen.

59. The protein complex of claim 58, wherein the T cell antigen is CD3, and the tumor-associated antigen is CEACAM5.

60. The protein complex of claim 58 or 59, wherein the first polypeptide comprises a sequence that is at least 80%, 90%, 95%, or 100% identical to SEQ ID NO: 2; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 40.

61. A protein complex, comprising:
(a) a first polypeptide comprising from N-terminus to C-terminus: optionally a first hinge region, a first CH2 domain, and a first CH3 domain;
(b) a second polypeptide comprising from N-terminus to C-terminus: a VH, a CH1 domain, optionally a second hinge region, a second CH2 domain, and a second CH3 domain; and
(c) a third polypeptide comprising from N-terminus to C-terminus: a single-domain antibody variable domain (VHH), optionally a linker peptide, a VL, and a CL domain,
wherein the VH and the VL associate with each other, forming an antigen binding site of a Fab that specifically binds to a T cell antigen, wherein the VHH specifically binds to a tumor-associated antigen.

62. The protein complex of claim 61, wherein the T cell antigen is CD3, and the tumor-associated antigen is CEACAM5.

63. The protein complex of claim 61 or 62, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 9; and the third polypeptide comprises a sequence that is at least 80%, 90%, 95%, or 100% identical to SEQ ID NO: 40.

64. A protein complex, comprising:
(a) a first polypeptide comprising from N-terminus to C-terminus: a single-domain antibody variable domain (VHH), optionally a first linker peptide, a VH, a CH1 domain, optionally a first hinge region, a first CH2 domain, and a first CH3 domain;
(b) a second polypeptide comprising from N-terminus to C-terminus: optionally a second hinge region, a second CH2 domain, and a second CH3 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

65. The protein complex of claim 64, wherein the T cell antigen is CD3, and the tumor-associated antigen is CEACAM5.

66. The protein complex of claim 64 or 65, wherein the first polypeptide comprises a sequence that is at least 80%, 90%, 95%, or 100% identical to SEQ ID NO: 41; the second polypeptide comprises a sequence that is at least 80%, 90%, 95%, or 100% identical to SEQ ID NO: 8; and the third polypeptide comprises a sequence that is at least 80%, 90%, 95%, or 100% identical to SEQ ID NO: 23.

67. A protein complex, comprising:
(a) a first polypeptide comprising from N-terminus to C-terminus: optionally a first hinge region, a first CH2 domain, and a first CH3 domain;
(b) a second polypeptide comprising from N-terminus to C-terminus: a single-domain antibody variable domain (VHH), optionally a second linker peptide, a VH, a CH1 domain, optionally a second hinge region, a second CH2 domain, and a second CH3 domain; and
(c) a third polypeptide comprising from N-terminus to C-terminus: a VL, and a CL domain,
wherein the VH and the VL associate with each other, forming an antigen binding site of a Fab that specifically binds to a T cell antigen, wherein the VHH specifically binds to a tumor-associated antigen.

68. The protein complex of claim 67, wherein the T cell antigen is CD3, and the tumor-associated antigen is CEACAM5.

69. The protein complex of claim 67 or 68, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 42; and the third polypeptide comprises a sequence that is at least 80%, 90%, 95%, or 100% identical to SEQ ID NO: 23.

70. A protein complex, comprising:
(a) a first polypeptide comprising from N-terminus to C-terminus: a VH, a CH1 domain, optionally a first hinge region, a first CH2 domain, a first CH3 domain, optionally a first linker peptide, and a single-domain antibody variable domain (VHH); and
(b) a second polypeptide comprising from N-terminus to C-terminus: a VL, a CL, optionally a second hinge region, a second CH2 domain, and a second CH3 domain,
wherein the VH and the VL associate with each other, forming an antigen binding site of a Fab that specifically binds to a T cell antigen, wherein the VHH specifically binds to a tumor-associated antigen.

71. The protein complex of claim 70, wherein the T cell antigen is CD3, and the tumor-associated antigen is CEACAM5.

72. The protein complex of claim 70 or 71, wherein the first polypeptide comprises a sequence that is at least 80%, 90%, 95%, or 100% identical to SEQ ID NO: 6; and the second polypeptide comprises a sequence that is at least 80%, 90%, 95%, or 100% identical to SEQ ID NO: 44.

73. A protein complex, comprising:
(a) a first polypeptide comprising from N-terminus to C-terminus: a VH, a CH1 domain, optionally a first hinge region, a first CH2 domain, and a first CH3 domain; and
(b) a second polypeptide comprising from N-terminus to C-terminus: a VL, a CL, optionally a second hinge region, a second CH2 domain, a second CH3 domain, optionally a second linker peptide, and a single-domain antibody variable domain (VHH),
wherein the VH and the VL associate with each other, forming an antigen binding site of a Fab that specifically binds to a T cell antigen, wherein the VHH specifically binds to a tumor-associated antigen.

74. The protein complex of claim 73, wherein the T cell antigen is CD3, and the tumor-associated antigen is CEACAM5.

75. The protein complex of claim 73 or 74, wherein the first polypeptide comprises a sequence that is at least 80%, 90%, 95%, or 100% identical to SEQ ID NO: 2; and the second polypeptide comprises a sequence that is at least 80%, 90%, 95%, or 100% identical to SEQ ID NO: 46.

76. A protein complex, comprising:
(a) a first polypeptide comprising from N-terminus to C-terminus: a VL, a CL, optionally a first hinge region, a first CH2 domain, a first CH3 domain, optionally a first linker peptide, and a single-domain antibody variable domain (VHH); and
(b) a second polypeptide comprising from N-terminus to C-terminus: a VH, a CH1 domain, optionally a second hinge region, a second CH2 domain, and a second CH3 domain,
wherein the VH and the VL associate with each other, forming an antigen binding site of a Fab that specifically binds to a T cell antigen, wherein the VHH specifically binds to a tumor-associated antigen.

77. The protein complex of claim 76, wherein the T cell antigen is CD3, and the tumor-associated antigen is CEACAM5.

78. The protein complex of claim 76 or 77, wherein the first polypeptide comprises a sequence that is at least 80%, 90%, 95%, or 100% identical to SEQ ID NO: 45; and the second polypeptide comprises a sequence that is at least 80%, 90%, 95%, or 100% identical to SEQ ID NO: 9.

79. A protein complex, comprising:
(a) a first polypeptide comprising from N-terminus to C-terminus: a VL, a CL, optionally a first hinge region, a first CH2 domain, and a first CH3 domain; and
(b) a second polypeptide comprising from N-terminus to C-terminus: a VH, a CH1 domain, optionally a second hinge region, a second CH2 domain, a second CH3 domain, optionally a second linker peptide, and a single-domain antibody variable domain (VHH),
wherein the VH and the VL associate with each other, forming an antigen binding site of a Fab that specifically binds to a T cell antigen, wherein the VHH specifically binds to a tumor-associated antigen.

80. The protein complex of claim 79, wherein the T cell antigen is CD3, and the tumor-associated antigen is CEACAM5.

81. The protein complex of claim 79 or 80, wherein the first polypeptide comprises a sequence that is at least 80%, 90%, 95%, or 100% identical to SEQ ID NO: 43; the second polypeptide comprises a sequence that is at least 80%, 90%, 95%, or 100% identical to SEQ ID NO: 13.

82. The protein complex of any one of claims 16-81, wherein the first CH3 domain comprises one or more knob mutations, and the second CH3 domain comprises one or more hole mutations.

83. The protein complex of any one of claims 16-82, wherein the VH comprises a sequence that is at least 80%, 90%, 95%, or 100% identical to any one of SEQ ID NOs: 25-31 and the VL comprises a sequence that is at least 80%, 90%, 95%, or 100% identical to any one of SEQ ID NOs: 32-38.

84. The protein complex of any one of claims 16-83, wherein the VHH comprises a sequence that is at least 80%, 90%, 95%, or 100% identical to SEQ ID NO: 18.

85. The protein complex of any one of claims 16-84, wherein the first hinge region and/or the second hinge region comprise a sequence that is at least 80%, 90%, 95%, or 100% identical to SEQ ID NO: 19.

86. The protein complex of any one of claims 16-85, wherein the first Fc region and/or the second Fc region comprise a sequence that is at least 80%, 90%, 95%, or 100% identical to SEQ ID NO: 20 or 21.

87. The protein complex of any one of claims 19-24 and 28-86, wherein the linker peptide, the first linker peptide, the second linker peptide and/or the third linker peptide comprises a sequence that is at least 80%, 90%, 95%, or 100% identical to SEQ ID NO: 15 or one or more repeats (e.g., 1, 2, 3, 4, 5, 6, 7, or 8) of SEQ ID NO: 16.

88. A nucleic acid comprising a polynucleotide encoding the antigen-binding protein of any one of claims 1-15, or the protein complex of any one of claims 16-87.

89. The nucleic acid of claim 88, wherein the nucleic acid is a DNA (e.g., cDNA) or RNA (e.g., mRNA).

90. A vector comprising one or more of the nucleic acids of claim 88 or 89.

91. A cell comprising the vector of claim 90.

92. The cell of claim 91, wherein the cell is a HEK293F cell or CHO cell.

93. A cell comprising one or more of the nucleic acids of claim 88 or 89.

94. A method of producing an antigen-binding protein or protein complex, the method comprising
(a) culturing the cell of any one of claims 91-93 under conditions sufficient for the cell to produce the antigen-binding protein or protein complex; and
(b) collecting the antigen-binding protein or protein complex produced by the cell.

95. An antibody-drug conjugate comprising the antigen-binding protein of any one of claims 1-15, or the protein complex of any one of claims 16-87, covalently bound to a therapeutic agent.

96. The antibody drug conjugate of claim 95, wherein the therapeutic agent is a cytotoxic or cytostatic agent.

97. A method of treating a subject having cancer, the method comprising administering a therapeutically effective amount of a composition comprising the antigen-binding protein of any one of claims 1-15, the protein complex of any one of claims 16-87, or the antibody-drug conjugate of claim 95 or 96, to the subject.

98. The method of claim 97, wherein the subject has a cancer expressing CEACAM5.

99. The method of claim 97 or 98, wherein the cancer is lung cancer, colorectal cancer, head and neck cancer, stomach cancer, pancreatic cancer, urothelial cancer, breast cancer, cervical cancer, or endometrial cancer.

100. A method of decreasing the rate of tumor growth, the method comprising contacting a tumor cell with an effective amount of a composition comprising the antigen-binding protein of any one of claims 1-15, the protein complex of any one of claims 16-87, or the antibody-drug conjugate of claim 95 or 96.

101. A method of killing a tumor cell, the method comprising contacting a tumor cell with an effective amount of a composition comprising the antigen-binding protein of any one of claims 1-15, the protein complex of any one of claims 16-87, or the antibody-drug conjugate of claim 95 or 96.

102. A pharmaceutical composition comprising the antigen-binding protein of any one of claims 1-15, or the protein complex of any one of claims 16-87, and a pharmaceutically acceptable carrier.


SEQUENCE LISTING (48 sequences)

SEQ ID NO: 1 - protein, 227 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 2 - protein, 450 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 3 - protein, 460 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 4 - protein, 347 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 5 - protein, 357 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 6 - protein, 580 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 7 - protein, 579 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 8 - protein, 227 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 9 - protein, 450 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 10 - protein, 460 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 11 - protein, 347 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 12 - protein, 357 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 13 - protein, 580 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 14 - protein, 580 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 15 - protein, 10 aa, synthetic construct
GGGGSGGGGS

SEQ ID NO: 16 - protein, 5 aa, synthetic construct
GGGGS

SEQ ID NO: 17 - protein, 223 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 18 - protein, 120 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 19 - protein, 12 aa, synthetic construct
ESKYGPPCPPCP

SEQ ID NO: 20 - protein, 215 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 21 - protein, 215 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 22 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 23 - protein, 216 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 15 aa, synthetic construct
GGGGSGGGGSGGGGS

SEQ ID NO: 25 - protein, 125 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 26 - protein, 119 aa, synthetic construct [sequence omitted]

SEQ ID NO: 27 - protein, 122 aa, synthetic construct [sequence omitted]

SEQ ID NO: 28 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 29 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 30 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 31 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 32 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIK

SEQ ID NO: 33 - protein, 106 aa, synthetic construct [sequence omitted]

SEQ ID NO: 34 - protein, 107 aa, synthetic construct [sequence omitted]

SEQ ID NO: 35 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 36 - protein, 106 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 37 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 38 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 39 - protein, 346 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 40 - protein, 346 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 580 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 42 - protein, 580 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 43 - protein, 443 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 44 - protein, 443 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 45 - protein, 573 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 46 - protein, 573 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 47 - protein, 122 aa, synthetic construct
QVQLQESGGGSVQAGGSLKLSCAASGRGFSRYAMGWFRQAPGQDREFVATISWTNSTDYADSVKGRFAISRDNAKNTAYLQMNSLKPEDTAVYYCAADKWASSTRSIDYDYWGQGIQVTVSS

SEQ ID NO: 48 - protein, 123 aa, synthetic construct
QVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS